(19) United States
(12) Patent Application Publication
(10) Pub. No.: US 2025/0197498 A1
(43) Pub. Date: Jun. 19, 2025

(54) METHODS FOR IN VIVO EXPANSION OF CD8+ T CELLS AND PREVENTION OR TREATMENT OF GVHD

(71) Applicant: CITY OF HOPE, Duarte, CA (US)

(72) Inventor: Defu ZENG, Arcadia, CA (US)

(73) Assignee: CITY OF HOPE, Duarte, CA (US)

(21) Appl. No.: 18/805,289

(22) Filed: Aug. 14, 2024

Related U.S. Application Data
(63) Continuation of application No. 16/543,472, filed on Aug. 16, 2019, now abandoned.
Application No. 16/543,472 is a continuation of application No. PCT/US2018/019524, filed on Feb. 23, 2018.
(60) Provisional application No. 62/462,853, filed on Feb. 23, 2017.

(51) Int. Cl. C07K 16/28; A61K 35/28; A61K 45/06; C07K 16/24

(52) U.S. Cl. CPC C07K 16/2812; A61K 35/28; C07K 16/246; A61K 45/06

(57) ABSTRACT

Disclosed herein are methods of preventing and treating acute GVHD and chronic GVHD after hematopoietic cell transplantation (HCT), as well as methods of in vivo augmenting expansion of donor CD8+ T cells in the lymphoid tissues in vivo after HCT and methods of augmenting recipient tissue expression of programmed death-ligand 1 (PD-L1, or B7H1) after HCT. The methods entail administering one or more doses of an effective amount of a therapeutic agent to a recipient simultaneously, immediately before, or immediately after HCT to temporarily deplete CD4+ T cells or to reduce serum IL-2. Some examples include an anti-CD4 antibody or an anti-CD4-meditope-immunotoxin, an anti-IL-2 antibody, an agent blocking IL-2R, and/or a PD-L1-Ig. One or more additional therapeutic agents such as IFN-γ can be administered.

### PRIORITY CLAIM

The present application is a continuation of U.S. application Ser. No. 16/543,472, filed Aug. 16, 2019, which is a continuation of International Application No. PCT/US2018/019524, filed Feb. 23, 2018 which claims the benefit of U.S. Provisional Application No. 62/462,853, filed Feb. 23, 2017, all of which are incorporated herein by reference in their entireties.

### STATEMENT OF GOVERNMENT INTEREST

The present invention was made with government support under Grant No. R01 AI066008, 2R56AI66008-11, R01 AI095239, and P30CA033572, awarded by the National Institutes of Health (NIH). The Government has certain rights in the invention.

### BACKGROUND

Allogeneic hematopoietic cell transplantation (HCT) is a curative therapy for hematological malignancies (i.e. leukemia and lymphoma), owing to graft versus leukemia/lymphoma (GVL) effects mediated by alloreactive T cells. These same T cells also mediate acute graft-versus-host disease (GVHD) and the subsequent development of chronic GVHD (1-9). Both alloreactive CD4⁺ and CD8⁺ T cells can mediate acute GVHD, and Th1 and Th17 cells play a critical role in initiating gut GVHD (10-14). While flow cytometry-sorted donor CD4⁺ T cells mediate severe GVHD through expression of FASL and production of proinflammatory cytokines (i.e. IFN-γ and TNF-α) (14-17), sorted donor CD8⁺ T cells prevent graft rejection and mediate GVL effects through their expression of perforin/granzyme, without causing acute clinical GVHD in several mouse models (2, 18, 19). However, the mechanisms whereby purified alloreactive CD8⁺ T cells mediate GVL effect without causing GVHD remains largely unknown.

Programmed death ligand 1 (PD-L1, also known as B7H1) functions as an immune checkpoint that interacts with PD-1 and CD80 (20, 21). PD-L1 is usually expressed by hematopoietic cells and by parenchymal cells under inflammatory cytokine (i.e. IFN-γ) induction (22). CD80 is constitutively expressed by T cells and is upregulated early after T cell activation (23), whereas PD-1 is expressed by T cells late after T cell activation (24). PD-L1 interaction with PD-1 induces anergy, exhaustion and apoptosis of activated T cells (25, 26); on the other hand, PD-L1/CD80 interaction has been reported to inhibit CD28/CTLA4 deficient T cell proliferation in vitro (21).

Expression of PD-L1 in recipient tissues decreases the severity of GVHD in conventional TBI-conditioned allogeneic recipients (27-29), while expression of PD-L1 by donor T cells increases the severity of GVHD by augmenting the expansion and survival of donor CD4⁺ and CD8⁺ T cells (30). It was shown that the interaction of PD-L1 with CD80 in the absence of PD-1 worsened GVHD by augmenting alloreactive CD4⁺ T cell proliferation and expansion, although simultaneous interactions of PD-L1 with both CD80 and PD-1 ameliorated GVHD by augmenting apoptosis of activated alloreactive CD4⁺ T cells (31).

Regulation of anergy, exhaustion, and apoptosis through PD-L1 interactions with CD80 and PD-1 on CD8⁺ T cells in allogeneic HCT has not yet been well characterized. It was shown that the absence of host tissue expression of PD-L1 contributed to expansion of infiltrating CD8⁺ T cells in GVHD target tissues in recipients with GVHD and lymphopenia (27). Other publications have shown that host tissue expression of PD-L1 caused exhaustion of alloreactive CD8⁺ T cells and reduced GVL effects in GVHD recipients (32, 33). However, it was reported that in vivo expansion of alloreactive CD8⁺ T cells in lymphoid tissues (i.e., spleen) early after HCT, before the onset of GVHD, was not affected by host tissue expression of PD-L1 (34).

The role of IFN-γ in acute GVHD pathogenesis remains controversial. IFN-γ is required for CD4⁺ T-mediated acute GVHD in the gut and liver by augmenting Th1 differentiation and up-regulating Th1 expression of gut and liver-homing chemokine receptors (α4β7, CCR9, CCR5 and CXCR3) (29, 43, 44). In contrast, as compared to CD4⁺ T cells, the same number of donor CD8⁺ Tc1 cells induced little gut acute GVHD (11, 64). IFN-γ-produced by CD8⁺ T cells is required to separate GVHD from GVL effects mediated by the CD8⁺ T cells, although IFN-γ does not directly kill tumor cells (65, 66).

IFN-γ is the key cytokine regulates tissue expression of programmed death-ligand 1 (PD-L1, also known as B7H1) (22, 67). Under non-inflammatory conditions, hematopoietic cells and lymphocytes constitutively express PD-L1 mRNA and protein, while parenchymal cells express PD-L1 mRNA without protein expression (22). Proinflammatory cytokines such as IFN-γ augment expression of PD-L1 mRNA and protein by hematopoietic cells, lymphocytes and parenchymal cells (22). Receptors for PD-L1 include CD80 and PD-1 (20, 21). PD-L1 interaction with its receptors PD-1 and CD80 induces anergy, exhaustion and apoptosis in activated T cells (25, 26). Previous studies have shown that recipient tissue expression of PD-L1 down-regulates GVHD in conventional TBI-conditioned allogeneic HCT, although the recipients still developed GVHD (29, 27, 28). It has been reported that interaction of PD-L1 with CD80 in the absence of PD-1 augmented acute GVHD by increasing alloreactive CD4⁺ T cell proliferation without increasing CD4⁺ T cell apoptosis, whereas simultaneous interactions of PD-L1 with both CD80 and PD-1 ameliorated GVHD by augmenting alloreactive CD4⁺ T cell proliferation and apoptosis (31).

Accordingly, there remain needs to improve in vivo expansion of CD8⁺ T cells and to prevent and treat not only acute GVHD but also chronic GVHD. This invention satisfies the needs in the art.

### SUMMARY

In one aspect, the disclosure provided herein relates to a method of augmenting expansion of donor CD8⁺ T cells in vivo after hematopoietic cell transplantation (HCT). The method entails administering one or more doses of an effective amount of a therapeutic agent to a recipient immediately before, during, or immediately after HCT to temporarily deplete CD4⁺ T cells or to temporarily reduce serum IL-2. In some embodiments, the therapeutic agent includes an anti-CD4 antibody or an anti-CD4-meditope-immunotoxin. In some embodiments, the anti-CD4⁺ antibody is a monoclonal antibody or a humanized antibody. In some embodiments, the therapeutic agent includes an anti-IL-2 antibody (e.g., an anti-IL-2 monoclonal antibody and/or humanized antibody) or an agent blocking IL-2R. In some embodiments, the CD8⁺ T cells are selectively expanded in lymphoid tissues but not in GVHD target tissues of the subject. In some embodiments, the expanded CD8⁺ T cells produce an increased amount of IFN-γ, comparing to control recipients received with IgG.

In another aspect, the disclosure provided herein relates to a method of preventing a subject from suffering from GVHD or treating a subject suffering from GVHD after HCT while preserving GVL. The method entails administering one or more doses of an effective amount of a therapeutic agent to a recipient simultaneously, immediately before, or immediately after HCT to temporarily deplete CD4⁺ T cells or to temporarily reducing serum IL-2. In some embodiments, the therapeutic agent includes, but is not limited to, an anti-CD4 antibody, an anti-CD4-meditope-immunotoxin, an anti-IL-2 antibody, or an IL-2R blocking agent. In some embodiments, the anti-CD4⁺ antibody is a monoclonal antibody or a humanized antibody. In some embodiments, acute GVHD is prevented or treated by administering to the subject a single dose of the therapeutic agent. In some embodiments, GVHD is prevented or treated by administering no more than three doses of the therapeutic agent. For example, the three doses are administered within one month, at one- or two-week intervals. In some embodiments, more than three doses of the therapeutic agent can be administered to prevent or treat GVHD. In some embodiments, one or more doses of PD-L1-Ig are administered to prevent or treat GVHD while preserving GVL. In some embodiments, the method further entails administration of one or more doses of IFN-γ to the subject in addition to temporarily depleting CD4⁺ T cells or reducing serum IL-2.

In another aspect, the disclosure provided herein relates to a method of preventing or treating GVHD and augmenting thymus recovery after HCT. The method entails administering one or more doses of an effective amount of a therapeutic agent to a recipient simultaneously, immediately before, or immediately after HCT to temporarily deplete CD4⁺ T cells from the transplant and from de novo generation or to temporarily reduce serum IL-2 for a period from 60 days to 120 days. In some embodiments, the therapeutic agent includes an anti-CD4 antibody, or an anti-CD4-meditope-immunotoxin. In some embodiments, the anti-CD4⁺ antibody is a monoclonal antibody or a humanized antibody. In some embodiments, the therapeutic agent includes an anti-IL2 antibody, or an agent blocking IL-2R. In some embodiments, the anti-IL2 antibody is a monoclonal antibody or a humanized antibody.

In another aspect, the disclosure provided herein relates to a method of augmenting recipient tissue expression of programmed death-ligand 1 (PD-L1, or B7H1) after HCT. The method entails administering one or more doses of an effective amount of a therapeutic agent to a recipient simultaneously, immediately before, or immediately after HCT. In some embodiments, the therapeutic agent includes an agent that temporarily depletes CD4⁺ T cells, such as an anti-CD4 antibody (e.g., a monoclonal or humanized anti-CD4 antibody) or an anti-CD4-meditope-immunotoxin. In some embodiments, the therapeutic agent includes an agent that temporarily reduces serum IL-2, such as an anti-IL-2 antibody (e.g., a monoclonal or humanized anti-IL-2 antibody) or an agent blocking IL-2R

### BRIEF DESCRIPTION OF THE DRAWINGS

The patent or application file contains at least one drawing executed in color. Copies of this patent or patent application with color drawing(s) will be provided by the Office upon request and payment of the necessary fee.

FIGS. 1A-1D show that small numbers of donor CD4+ T cells augment survival of donor CD8+ T cells in GVHD target tissues in an IL-2 dependent manner. FIG. 1A shows that lethally irradiated BALB/c recipients were transplanted with C57BL/6 TCD-BM (2.5×10⁶) together with either splenocytes (5×10⁶) or ex vivo CD4⁺ T cell-depleted splenocytes that contained the same number of CD8⁺ T cells as present in 5×10⁶ whole spleen cells. The recipients of whole spleen cells were injected with depleting anti-CD4 mAb (500 ug/mouse) at the time of HCT to in vivo deplete donor CD4⁺ T cells. The donor splenocytes before HCT and splenocytes from the recipients 7 days after HCT were analyzed for the percentage and yield of donor CD4⁺ T cells. Representative patterns and means±SEM of the percentage and yield of donor CD4⁺ T cells in the spleen are shown. Mean±SEM; n=4 per group. FIG. 1B shows that lethally irradiated BALB/c recipients were injected with TCD-BM (2.5×10⁶) alone, TCD-BM plus flow cytometry-sorted CD4⁺ T cells (0.075×10⁶) alone, TCD-BM plus sorted CD8⁺ T cells (1×10⁶) alone, or TCD-BM plus both CD4⁺ and CD8⁺ T cells. Recipients were monitored for clinical signs of GVHD such as diarrhea and survival. Percentage of mice without diarrhea and percent mice died in association with diarrhea are shown; n=6-8 per group combined from two replicate experiments. FIG. 1C shows yield of donor CD8⁺ T cells and percentage of Annexin V⁺ donor CD8⁺ T cells in the spleen and colon in recipients transplanted with CD8⁺ T cells alone or with CD4⁺ T cells. N=4-6 per group. FIG. 1D shows that lethally irradiated BALB/c recipients were injected with CD8⁺ T (1×10⁶) and CD4⁺ T cells (0.075×10⁶) and TCD-BM, and then injected IP with control rat IgG or anti-IL-2 (500 μg/mouse) on days 0, 2, 4 and 6 after HCT. On day 7, spleen and colon tissue infiltrating CD8⁻ T cells were analyzed for apoptosis. N=4-5. Data represent mean±SE combined from two replicate experiments. P values were calculated by unpaired two-tailed Student t tests (1A, 1C and 1D) or log-rank test (1B) (*p<0.05, **p<0.01, ***p<0.001, ****p<0.0001).

FIGS. 2A and 2B show that a single injection of anti-CD4 mAb after HCT prevents acute but not chronic GVHD, with C57BL/6 donors and BALB/c recipients. Lethally irradiated BALB/c recipients transplanted with splenocytes (5×10⁶) and TCD-BM (2.5×10⁶) from C57BL/6 donors were given a single i.v. injection of rat-IgG or anti-CD4 mAb (500 μg/mouse) at the time of HCT. Recipients given TCD-BM (2.5×10⁶) alone were used as controls. Recipients were monitored for clinical signs of GVHD, including body weight change, diarrhea, hair loss, and survival († indicates death of all recipients in a group). FIG. 2A shows percentage of body weight change, percentage of recipients without diarrhea, clinical cutaneous GVHD score, and percentage of surviving recipients. N=8 per group combined from two replicate experiments. FIG. 2B shows that at 7 days after HCT, skin, salivary gland, lung, liver, small intestine (Sm. Int.) and colon tissues were evaluated for histopathologic evidence of GVHD. Representative photomicrographs are shown (original magnification×200), n=6 per group. Arrows indicate the changes in GVHD recipients as compared with control recipients. Data represent mean±SE combined from two replicate experiments. P values were calculated by unpaired two-tailed Student t tests (2B) or log-rank test (2A) (*p<0.05, **p<0.01, ***p<0.001).

FIG. 3 shows recovery kinetics of CD4⁺ T cells after a single anti-CD4 mAb treatment. Lethally irradiated BALB/c recipients transplanted with splenocytes (2.5×10⁶) and TCD-BM (2.5×10⁶) from C57BL/6 donors were given a single i.v. injection of rat-IgG or anti-CD4 mAb (500 μg/mouse) at the time of HCT. On days 5, 7, 14, 21 and 28 after HCT, splenocytes from recipients were analyzed for CD4⁺ T cells expansion and recovery. Representative patterns and mean±SEM of the percentage and yield of CD4⁺ T cells in the spleen are shown. Mean±SEM; n=4 for each group at each time point.

FIGS. 4A-4C show that three injections of anti-CD4 mAb prevented both acute and chronic GVHD. Lethally irradiated BALB/c recipients transplanted with splenocytes (2.5×10⁶) and TCD-BM (2.5×10⁶) from C57BL/6 donors were given 1 to 3 i.v. injections of rat-IgG or anti-CD4 mAb (500 μg/mouse) at days 0, 14 and 28 after HCT. Recipients given TCD-BM (2.5×10⁶) alone were used as controls. Mice were monitored for clinical signs of GVHD and survival. FIG. 4A shows percentage of body-weight change, percentage of recipients without diarrhea, clinical cutaneous GVHD score, and percent survival; n=8 per group combined from two replicate experiments. FIG. 4B shows that at 50-60 days after HCT, histopathology of skin, salivary gland, lung, liver, small intestine and colon was evaluated. A representative photomicrograph (original magnification 200×) and means±SEM of histopathology scores are shown; n=6 per group. Arrows indicate the changes in GVHD recipients as compared with control recipients. FIG. 4C shows that at day 50-60 and day 100 after HCT, spleens were harvested from recipients, stained with anti-H-2K^{b}, TCRβ, CD4 and CD8 mAbs and analyzed for CD4⁺ T cells recovery after anti-CD4 mAb treatment. A representative panel from 1 of 4 recipients in each group is shown. Data represent mean±SEM combined from two replicate experiments. P values were calculated by unpaired two-tailed Student t tests (4B) or log-rank test (4A) (*p<0.05, **p<0.01, ***p<0.001).

FIGS. 5A-5D show that depletion of donor CD4⁺ T cells allowed thymic epithelial cell regeneration. Lethally irradiated BALB/c recipients received HCT and anti-CD4 or rat-IgG treatment as described in FIG. 7. Recipients given TCD-BM were used as controls. The percentage and yield of CD4⁺CD8⁺ (DP) thymocytes were kinetically measured on days 7, 14, 21, 28, 45 and 60 days after HCT. Percentage, yield, and histoimmunofluorescent staining of mTEC were measured on day 45. FIG. 5A shows the kinetic analysis of DP thymocytes. A representative flow cytometry pattern is shown from 1 of 4 replicate experiments; Mean±SE of DP percentage among total thymocytes and yield is shown. FIG. 5B shows that on day 45 after HCT, percentage and yield of CD4⁺CD8⁺ thymocytes were measured and compared via flow cytometry analysis. FIG. 5C shows that on day 45 after HCT, percentage of mTEC was measured and compared via flow cytometry analysis. Representative patterns and Mean±SE (N=4) are shown (*p<0.05, **p<0.01, ***p<0.001). FIG. 5D shows histoimmunofluorescent staining of mTEC and cTEC, using cytokeratin 8 (red, cTEC) and UEA-I (green, mTEC). A representative photomicrograph from each group is shown from 1 of 4 replicate experiments (original magnification 200×).

FIGS. 6A-6E show that three injections of anti-CD4 mAb prevented both acute and chronic GVHD and preserved GVL effects after HCT with C57BL/6 donors and BALB/c recipients. Lethally irradiated BALB/c recipients transplanted with splenocytes (5×10⁶) and TCD-BM (2.5×10⁶) from C57BL/6 donors. Recipients were challenged with i.p. injection of BCL1/Luc cells (5×10⁶/mouse) and were given 3 i.v. injections of rat-IgG or anti-CD4 mAb (500 μg/mouse) at days 0, 14 and 28 after HCT. Recipients given TCD-BM cells (2.5×10⁶) alone were used as controls. Mice were monitored for tumor growth using in vivo bioluminescent imaging (BLI), clinical signs of GVHD and survival. FIG. 6A shows a representative BLI image from each time point for each group. FIG. 6B shows a summary of photons/sec of recipients. FIG. 6C shows clinical GVHD score. FIG. 6D shows % of survival. FIG. 6E shows serum AST concentrations on days 7 and 12 after HCT. n=4-8 per group, combined from two replicate experiments. Data represent mean±SE. P values were calculated by multiple t test (6B, 6C) or log-rank test (6D) or unpaired two-tailed Student t tests (6E) (*p<0.05, **p<0.01, ***p<0.001).

FIGS. 7A to 7E show that depletion of donor CD4⁺ T cells preserved GVL effect while preventing GVHD after HCT with A/J donors and C57BL/6 recipients. Lethally irradiated C57BL/6 recipients transplanted with splenocytes (10×10⁶, 20×10⁶ or 40×10⁶) and BM cells (10×10⁶) from A/J donors. eGFP positive Blast-Crisis Chronic Myelogenous Leukemia cells (eGFP⁺BC-CML, 20×10³) were injected i.v. on day 0. Recipients were injected with either rat IgG or anti-CD4 mAb (500 μg/mouse) at days 0, 7, 14, 28, 45 and 60 after HCT. Recipients were monitored for signs of tumor burden and clinical GVHD. Data are combined from 2-4 replicate experiments. FIG. 7A shows percentage of survival; n=8-16 per group. FIG. 7B shows that moribund mice with or without GVHD during observation and mice at day 100 after HCT were checked for BC-CML tumor cells in the spleen, liver and bone marrow. Percentage of BC-CML cells in spleen, liver and bone marrow is shown; n=6-12 per group. FIG. 7C shows that 100 days after HCT, splenocytes were stained with anti-H-2K^{b}, TCRβ, CD4 and CD8 mAbs and analyzed for CD4⁺ T cell recovery after anti-CD4 mAb treatment. One representative panel from four recipients in each group is displayed. FIG. 7D shows percentage of body-weight change in recipients transplanted with 40×10⁶ splenocytes treated with either rat IgG or anti-CD4 antibody; n=8-12 per group. FIG. 7E shows that 100 days after HCT, histopathology of skin, salivary gland, lung, liver (original magnification 200×), small intestine and colon (original magnification 400×) was evaluated. A representative photomicrograph and mean±SEM of histopathology scores are shown; n=6 per group. Data represent mean±SE combined from 2-4 independent experiments. P values were calculated by log-rank test (7A) or unpaired two-tailed Student t tests (7B, 7E) or multiple t test (7D), (*p<0.05, **p<0.01, ***p<0.001).

FIGS. 8A and 8B show that depletion of donor CD4⁺ T cells immediately after HCT preserved GVL in C57BL/6 recipients after transplantation from A/J donors and challenge with GVL-resistant BC-CML cells. FIG. 8A shows that lethally irradiated C57BL/6 mice were transplanted and treated as in FIG. 7. Spleen, liver and bone marrow were harvested from recipients when they were moribund (10×10⁶ BM alone or with 10×10⁶ spleen) or 100 days after HCT (40×10⁶ spleen with 10×10⁶ BM). Percentages of eGFP⁺ BC-CML cell are shown. n=6-12 per group. FIG. 8B shows that lethally irradiated C57BL/6 mice were transplanted with 10×10⁶ BM with 40×10⁶ spleen or CD8⁺ T cells-depleted spleen contained the same number of mononuclear cells as 40×10⁶ spleen from A/J donor, i.v. anti-CD4 mAb (500 μg/mouse) on day 0, 7, 14, 28, 45 and 60 after HCT. Recipients were monitored for tumor growth and survival. Percentages of survival are shown. n=8 per group combined from two replicate experiments.

FIGS. 9A-9C show that depletion of donor CD4⁺ T cells preserved GVL effect while preventing GVHD in a xenogeneic GVHD model. NSG recipients transplanted with PBMC (20×10⁶ i.p.) from healthy human donors were injected with either IgG or anti-human CD4 mAb (200 μg/mouse, twice weekly for 4 weeks). 1×10⁶ eGFP⁺ Raji cells were injected i.p. on day 0. Recipients were monitored for signs of tumor burden and clinical GVHD. FIG. 9A shows percentage of body weight change, survival and representative photograph of mice transplanted with 20×10⁶ PBMC at day 50-60 after HCT; n=12 per group. FIG. 9B shows that 50-100 days after HCT, histopathology of skin, salivary gland, lung, liver was evaluated. Tissues from IgG-treated group were harvested˜50 days after HCT when the recipients had become moribund. Tissues from anti-CD4-treated recipients were harvested at 100 days after HCT when the experiments ended. A representative photomicrograph (original magnification 200×) and mean±SEM of histopathology scores are shown; n=6 per group. FIG. 9C shows survival of recipients transplanted with 20×10⁶ PBMC and 1×10⁶ Raji cells with IgG or anti-human CD4 mAb; n=12 per group. Panels show eGFP staining to identify Raji cells in the spleen, liver and BM with or without anti-CD4 treatment when mice became moribund or at day 100 after HCT when the experiments ended. Percentage of Raji cells in spleen, liver and BM are shown; n=4 per group. Data represent mean±SE combined from two replicate experiments. P values were calculated by unpaired two-tailed Student t tests (9B, 9C) or multiple t test and log-rank test (9A, 9C) (*p<0.05, **p<0.01, ***p<0.001).

FIG. 10 shows that with 1 of 4 human PBMC donors, anti-CD4 mAb treatment only partially prevented xenogeneic GVHD. NSG recipients were transplanted with human PBMC as in FIG. 9. Recipients were monitored for clinical GVHD and survival. Percentage of bodyweight change and percentage of survival is shown. n=4 per group.

FIGS. 11A-11D: FIGS. 11A and 11B show that depletion of donor CD4⁺ T cells increased serum IFN-γ concentrations but decreased IL-2 concentrations and augmented CD8⁺ T cell expansion in lymphoid tissues but not in GVHD target tissues. BALB/c recipients transplanted with splenocytes (2.5×10⁶) and TCD-BM cells from C57BL/6 donors were injected with either rat IgG or anti-CD4 mAb (500 μg/mouse) at day 0 after HCT. FIG. 11A shows concentrations of IFN-γ, IL-2 and TNF-α in serum from recipients 7 days after HCT; n=6 per group. FIG. 11B shows splenocytes from recipients at day 7 after HCT were gated on H-2K^{b+}TCRβ⁺ and displayed as IFN-γ versus CD4 or CD8. Representative patterns and Mean±SEM of the percentage and yield of IFN-γ⁺ donor T cells in the spleen are shown; n=8 per group. FIGS. 11C and 11D show the kinetic changes of donor CD8⁺ T cell expansion and infiltration. At days 5, 7, 10, 14, 21, and 28 days after HCT, spleen, PLN, MLN, liver, lung and colon of recipients were harvested for analysis of donor CD8⁺T yield. Mean±SEM of the yield of H-2Kb⁺TCRβ⁺CD8⁺ T cells are shown; n=4-6 per group. Data represent mean±SE combined from two replicate experiments. P values were calculated by unpaired two-tailed Student t tests (*p<0.05, **p<0.01, ***p<0.001).

FIGS. 12A and 12B show injected and de novo generated T cells in IgG- or anti-CD4-treated recipients at 28 days after HCT. Purified thy1.2⁺CD45.2⁺ T cells (1×10⁶) and CD45.1⁺TCD-BM cells (2.5×10⁶) were transplanted into lethally irradiated BALB/c recipients. 28 days after HCT, spleen T cells were analyzed with flow cytometry for CD45.2, CD45.1, and Foxp3. One representative pattern is shown for two replicate experiments. N=4. Data represent mean±SE combined from two replicate experiments. P values were calculated by unpaired two-tailed Student t tests (****p<0.0001).

FIGS. 13A and 13B show that in vivo depletion of CD4⁺ T cells did not affect donor CD8⁺ T cell homing and chemokine receptor expression. Lethally irradiated BALB/c recipients transplanted with splenocytes (2.5×10⁶) and TCD-BM (2.5×10⁶) from C57BL/6 donors were given one i.v. injection of rat-IgG or anti-CD4 mAb (500 μg/mouse) at the time of HCT. At day 7 after HCT, spleen, mesenteric lymph nodes (MLN), small intestine (Sm. Int) and colon of recipients were harvested. FIG. 13A shows expression of CCR9, CXCR3 and α4β7 on donor CD8⁺T cells of MLN. Mean±SE; n=4 per group from 2 replicate experiments. FIG. 13B shows that expression of chemokine mRNA in Sm. Int and colon was measured by real-time RT-PCR. Expression of CCL25 in Sm. Int. tissue and CXCL9, CXCL10, CXCL11 in colon tissue relative to the house keeping gene GAPDH are shown. Mean±SE; N=6 per group. Data represent mean±SE combined from two replicate experiments. P values were calculated by unpaired two-tailed Student t tests (*p<0.05, **p<0.01).

FIGS. 14A-14E show that depletion of donor CD4⁺ T cells protected Paneth cells, colonic epithelial cells and hepatocytes. Lethally irradiated WT BALB/c recipients transplanted with TCD-BM alone or with TCD-BM cells and splenocytes (2.5×10⁶) from C57BL/6 donors were injected with rat-IgG or anti-CD4 mAb (500 μg/mouse) on day 0. Seven days after HCT, intestinal and liver tissue were analyzed. FIG. 14A shows that small intestine paraffin sections were stained with anti-IL-22R (green), anti-lysozyme (red), and DAPI (blue). FIG. 14B shows that colon paraffin sections were stained with anti-cytokeratin (CK) and DAPI (blue). For FIGS. 14A and 14B, one representative photomicrograph (original magnification 400×) is shown from 4/group. FIG. 14C shows that liver enzymes in serum of recipients at days 7, 10 and 21 were measured. Mean±SEM; n=4-6 per group. (*p<0.05, **p<0.01, ***p<0.001). FIG. 14D shows tunel staining for hepatocyte apoptosis assay. A representative immunofluorescent photomicrograph (original magnification 400×) and mean±SEM of percentage of Tunel⁺ apoptotic hepatocytes are shown, n=4 per group. FIG. 14E shows that recipients were sacrificed at day 21 after HCT and sorted liver-infiltrating donor CD8⁺ T cells (1×10⁶) were transplanted together with TCD-BM (5×10⁶) into secondary 200 cGy-irradiated Rag2^{-/-} BALB/c mice. Mice were monitored for clinical GVHD. Percentage of body weight change, clinical cutaneous GVHD score, survival curve and representative photo of mice at D60 after HCT are shown; n=8 per group combined from two replicate experiments. P values were calculated by unpaired two-tailed Student t tests (14C) and multiple t test and log-rank test (14E) (*p<0.05, **p<0.01, ***p<0.001).

FIGS. 15A-15E show that depletion of donor CD4⁺ T cells augmented donor CD8⁺ T cell apoptosis in the intestine and anergy/exhaustion in the liver, but not in the spleen. Lethally irradiated WT BALB/c mice were transplanted and treated at day 0 with IgG or anti-CD4 mAb as in FIG. 14. On day 7 after HCT, spleen, liver and colon from recipients were harvested. FIG. 15A shows yield, Annexin V staining and BrdU staining of donor CD8⁺ T cells in spleen, liver and colon; n=4-6 per group. FIGS. 15B and 15C show Grail, Tim3 and IL7Rα expression by donor CD8⁺ T cells in spleen and liver; n=4-6 per group. FIGS. 15D and 15E show percentage of Eomes⁺T-bet⁺ and Eomes⁺PD1⁺ donor CD8⁺ T cells in spleen and liver; n=4 per group. Data represent mean±SE combined from two replicate experiments. P values were calculated by unpaired two-tailed Student t test (*p<0.05, **p<0.01, ***p<0.001).

FIGS. 16A-16C show representative flow cytometry patterns. A representative panel from 1 of 4 recipients in each group is shown.

FIGS. 17A-17E show that depletion of donor CD4⁺ T cells augmented donor CD8⁺ T anergy/exhaustion in the liver, but not in the spleen on day 10 after HCT. Lethally irradiated WT BALB/c mice were transplanted and treated at day 0 with IgG or anti-CD4 mAb as in FIG. 14. On day 10 after HCT, spleen and liver from recipients were harvested. FIG. 17A shows yield and BrdU and Annexin V staining of CD8⁺ T cells in spleen and liver; n=4-8 for each group from 2 replicate experiments. FIGS. 17B and 17C show Grail, Tim3 and IL7Rα expression by donor CD8⁺ T cells in spleen and liver; n=5 for each group from 2 replicate experiments. FIGS. 17D and 17E show percentages of Eomes⁺T-bet⁺ and Eomes⁺PD1⁺ donor CD8⁺ T cells in spleen and liver; n=4 for each group. Data represent mean±SE combined from two replicate experiments. P values were calculated by unpaired two-tailed Student t tests (*p<0.05, **p<0.01, ***p<0.001).

FIG. 18 shows serum IL-27 concentrations in Rat IgG- or anti-CD4-treated recipients. HCT was performed as in FIG. 11, and 7 days after HCT, serum IL-27 concentrations were measured by ELISA. Mean±SE of 4 replicate experiments is shown.

FIGS. 19A-19C show that anti-CD4 treatment failed to prevent acute GVHD in recipients given IFN-γ^{-/-} donor transplants. Lethally irradiated BALB/c recipients transplanted with splenocytes (5×10⁶) and TCD-BM (2.5×10⁶) from wild-type or IFN-γ^{-/-} C57BL/6 donors, and then given a single i.v. injection of anti-CD4 mAb (500 μg/mouse) at the time of HCT. Recipients were monitored for clinical signs of GVHD, including body weight change, diarrhea, hair loss, and survival. FIG. 19A shows percentage of body weight change, percentage of recipients without diarrhea, clinical cutaneous GVHD score, and percentage of surviving recipients. n=10 per group combined from two replicate experiments. FIG. 19B shows that 7 days after HCT, spleen and liver CD8⁺ T, CD11c⁺ DC and Mac-1/Gr-1⁺ myeloid cells were analyzed for surface PD-L1. One representative pattern is shown of 6 replicate experiments. FIG. 19C shows mean±SE of PD-L1 MFI, n=6. Data represent mean±SE combined from two replicate experiments. P values were calculated by unpaired two-tailed Student t tests. (*p<0.05, **p<0.01, *p<0.001, ****p<0.0001).

FIGS. 20A-20D show that depletion of donor CD4⁺ T cells prevented liver damage and protected Paneth cells and colon epithelial cells through a mechanism that depended on PD-L1 expression in host tissue. WT or PD-L1^{-/-} BALB/c recipients transplanted with splenocytes (5×10⁶) and TCD-BM cells from C57BL/6 donor were injected with anti-CD4 mAb (500 μg/mouse) on day 0; as a control, WT BALB/c recipients were injected with rat-IgG (500 μg/mouse) on day 0 and transplanted with splenocytes and TCD-BM. Recipients were monitored for clinical signs of acute GVHD and survival († indicates death of all recipients in a group). FIG. 20A shows that colon epithelial cells from recipients 7 days after HCT were stained with anti-CK, CD45 and PD-L1 mAbs; representative pattern of PD-L1 expression on CK⁺CD45⁻ colonial epithelial cells and MFI of PD-L1 are shown (n=4 per group, Mean±SEM). FIG. 20B shows percentage of body weight change, percentage of recipients without diarrhea and percentages of surviving recipients. n=8 per group from two replicate experiments. FIG. 20C shows serum alanine aminotransferase (ALT), aspartate aminotransferase (AST) and albumin (ALB) concentrations on day 7 in WT and PD-L1^{-/-} recipients treated with anti-CD4 mAb (n=6 per group from 3 experiments, Mean±SEM); Tunel staining for hepatocyte apoptosis assay. A representative Immunofluorescent photomicrograph (original magnification 400×) is shown (n=4 per group; Mean±SEM). FIG. 20D shows that immunofluorescent staining was performed on small intestine and colon as described in FIG. 14. A representative photomicrograph (original magnification 400×) is shown (n=4 per group). Data represent mean±SE combined from 2-3 independent experiments. P values were calculated by unpaired two-tailed Student t tests (*p<0.05, **p<0.01, ***p<0.001).

FIG. 21 shows that similar to CD4⁺ T cells, CD8⁺ T cells induced lethal GVHD in PD-L1^{-/-} recipients. Lethally irradiated WT and PD-L1^{-/-} BALB/c recipients were injected with purified CD4⁺ or CD8⁺ T cells (2.5×10⁶and 5×10⁶) and TCD-BM (2.5×10⁶) from C57BL/6 donors. Recipient survival was compared for up to 30 days. N=8 combined from two replicate experiments.

FIGS. 22A-22C show that depletion of donor CD4⁺ T cells allows host-tissue PD-L1 to tolerize CD8⁺ T cells in GVHD target tissues but not in lymphoid tissues. Lethally irradiated WT or PD-L1^{-/-} BALB/c mice were transplanted and treated at day 0 with anti-CD4 mAb as described in FIG. 14. On day 7 after HCT, spleen, liver and colon from recipients were harvested. FIG. 22A shows yield, Annexin V staining and BrdU staining of donor CD8⁺ T cells in SPL, liver and colon; n=4-6 per group. FIG. 22B shows Grail, Tim3 and IL7Rα expression by donor CD8⁺ T cells in spleen and liver; n=4-6 per group. FIG. 22C shows percentage of Eomes⁺T-bet⁺ cells and Eomes⁺PD1⁺ cells among donor CD8⁺ T cells in spleen and liver; n=4 per group. Data represent mean±SE combined from two replicate experiments. P values were calculated by unpaired two-tailed Student t tests (*p<0.05, **p<0.01, ***p<0.001).

FIGS. 23A-23D show representative flow cytometry patterns. A representative panel from 1 of 4-6 recipients in each group is shown.

FIG. 24 shows that seven days after HCT, CD8⁺ T cells from the liver were analyzed for their expression of IL-7Rα, Eomes, T-bet, and PD-1. Mean±SE of MFI is shown for 4 replicate experiments. HCT was set up as described in FIG. 6.

FIGS. 25A-25D show that blocking anti-PD-L1 treatment led to xenogeneic GVHD in anti-CD4-treated NSG mice. FIG. 25A shows that human CD8⁺ T cells bind to mouse PD-L1-Ig. FIG. 25B shows that PBMC from 3 healthy donors were distributed into 15 NSG mice with 5 mice/donor and 20×10⁶ PBMC/mouse. All mice were treated with anti-human CD4 (200 μg/mouse, twice weekly for 4 weeks), and 9 mice (groups of 3 mice given cells from each of the 3 donors) were treated with anti-mouse PD-L1 (5 μg/g body weight, twice weekly for 4 weeks), and the remaining 6 mice (groups of 2 mice given cells from each of the 3 donors) were treated with control IgG. Mice were monitored for clinical signs of GVHD, bodyweight and survival. All anti-PD-L1-treated mice showed bodyweight loss and died by 80 days after HCT, while control mice showed no signs of GVHD. FIGS. 25C and 25D show that 60 days after HCT, moribund GVHD mice and control GVHD-free mice were analyzed for CD8⁺ T cell percentage and yield in the liver and lung as well as CD8⁺ T expression of PD-1. A representative flow cytometry patterns of 1 of 4 mice in each group and mean±SE of MFI and yield of CD8⁺ T cells from the liver and lung are shown. P values were calculated by unpaired two-tailed Student t tests (*p<0.05, *p<0.01, *p<0.001).

FIGS. 26A-26E show that donor CD8⁺ T-T PD-L1/CD80 interactions augmented CD8⁺ T expansion and GVL effects in lymphoid tissues. FIG. 26A shows that lethally irradiated WT BALB/c recipients received HCT as described in FIG. 14. PD-L1, PD1 and CD80 expression on donor CD8⁺ T cells in spleen, liver and colon on day 7 after HCT; n=4-6 per group. FIG. 26B shows that WT BALB/c recipients were transplanted with 1×10⁶ Thy1.2⁺ splenocytes from WT or PD-L1^{-/-} C57BL/6 donors and TCD-BM cells from WT C57BL/6 and given anti-CD4 mAb (500 μg/mouse) on day 0. Yield, Annexin V staining, Bcl-xl staining, % Eomes⁺PD1⁺ cells among donor CD8⁺ T cells in spleen are shown; n=6-10 per group, combined from two replicate experiments. FIG. 26C shows that 1×10⁶ Thy1.2⁺ splenocytes from CD80^{-/-} donors were used to repeat experiments described in FIG. 26B; n=8. FIG. 26D shows that anti-CD4 treated WT BALB/c recipients were injected with IgG or PD-L1-specific mAb 43H12 (500 μg/mouse) on days 0 and 2 after HCT. Yield, Annexin V staining, Bcl-xl staining, and % Eomes⁺PD1⁺ cell among donor CD8⁺ T cells in spleen are shown; n=4-6 per group. FIG. 26E shows that anti-CD4 treated BALB/c recipients of splenocytes, TCD-BM and BCL1/Luc⁺ cells were treated with 43H12 mAb or control IgG on days 0 and 2. Recipients were monitored for survival and tumor burden as described in FIG. 6. A representative BLI for each group, photons/sec of BLI, survival and % Bcl-1 cells in the spleen, mesenteric lymph nodes, liver and lung are shown, n=4-8 per group. Data represent mean±SE combined from two replicate experiments. P values were calculated by unpaired two-tailed Student t tests (*p<0.05, **p<0.01, ***p<0.001, ****p<0.0001).

FIGS. 27A-27D show representative flow cytometry patterns. A representative panel from 1 of 4-6 recipients in each group is shown.

FIGS. 28A-28B show non-T hematopoietic cell expression of PD-L1 and CD80. Seven days after HCT, donor-type CD11c⁺ DCs and Mac-1/Gr-1⁺ myeloid cells from the spleen, liver and colon were analyzed for expression of PD-L1 and CD80. Mean±SE of MFI combined from 3 replicate experiments is shown. P values were calculated by unpaired two-tailed Student t tests (*p<0.05, **p<0.01, ***p<0.001, ****p<0.0001).

FIG. 29 is a diagram of donor and host tissue cell expression of PD-L1 in regulating donor CD8+ T expansion and tolerance in the lymphoid and GVHD target tissues.

FIGS. 30A and 30B show that in vivo depletion of donor CD8⁺ T cells did not protect host thymus after HCT. Lethally irradiated BALB/c recipients transplanted with splenocytes (2.5×10⁶) and TCD-BM from C57BL/6 donors were given anti-CD8 mAb (200 μg/mouse) at day 0, 3 and 6. FIG. 30A shows that splenocytes of recipients on day 7 after HCT were stained with anti-H-2k^{b}, TCRβ, CD4 and CD8β mAbs. One representative pattern of CD4 and CD8 percentage of donor T cells is shown. FIG. 30B shows kinetic analysis of CD4⁺CD8⁺ thymocytes at days 7, 14, 21 and 28 after HCT. One representative pattern is shown of 4 replicate experiments.

FIG. 31 shows that depletion of donor CD4⁺ T cells increased donor MNCs, total T and CD8⁺ T cells in the spleen. Lethally irradiated BALB/c recipients transplanted with splenocytes (2.5×10⁶) and TCD-BM from C57BL/6 donors were given either rat-IgG or anti-CD4 mAb (500 μg/mouse) at day 0. At day 7 after HCT, spleen tissues were harvested for FACS analysis. Splenocytes were stained with anti-H-2k^{b}, TCRβ, and CD8α mAbs. Representative patterns and means±SE of yield of mononuclear (MNCs), total T cells, and CD8⁺ T cells are shown. N=4 from 2 replicate experiments. Unpaired two-tailed Student t tests were used to compare means (*p<0.05, **p<0.01, ***p<0.001).

FIG. 32A-32C show that depletion of donor CD4⁺ T cells augmented donor CD8⁺ T cell proliferation and expansion in the spleen without increase of anergy or apoptosis, which is independent of recipient tissue PD-L1. Lethally irradiated WT or PD-L1^{-/-} BALB/c recipients received HCT and anti-CD4 treatment as described in FIG. 7. Seven days after HCT, donor CD8⁺ T cells in recipient spleen were analyzed for anergy and exhaustion related surface markers as well as measured for proliferation and apoptosis. FIG. 32A shows gated donor CD8⁺ T cells in histogram of CD80, PD-1, GRAIL, IL-7Rα, and TIM3. One representative flow cytometry patterns and mean±SE of MFI are shown; N=6. FIG. 32B shows gated donor CD8⁺ T cells in CD8 versus BrdU; and gated CD8⁺ T cells in histogram of Annexin V. One representative flow cytometry patterns and mean±SE (N=6) of MFI, percentage of BrdU⁺, or Annexin V⁺ cells are shown in FIGS. 12A and 12B (*p<0.05, **p<0.01, ***p<0.001). FIG. 32C shows mean±SE (N=6) of the yield of donor CD8⁺ T cells in the spleen.

FIGS. 33A-33C show evaluation of donor CD8⁺ T expansion and apoptosis in the colon tissues. Representative flow patterns and Mean±SE of MFI or cell numbers are shown; N=6, combined from 3 replicate experiments (*p<0.05, **p<0.01, ***p<0.001). FIG. 33A shows CD80 and PD-1 expression on colonial donor CD8⁺ T cells. FIG. 33B shows that colonial donor CD8⁺ T cells of BrdU-treated recipients are first shown in anti-CD8 versus anti-BrdU; Gated CD8⁺ T cells are also shown in histogram of Annexin V. FIG. 33C shows the yield of donor CD8⁺ T cells from colon tissue.

FIGS. 34A-34D shows that depletion of donor CD4⁺ T cells protected hepatocytes and augmented anergy and exhaustion of liver infiltrating CD8⁺ T cells. FIG. 34A shows representative histogram and mean±SE of MFI for CD80, PD-1, GRAIL, IL7Rα, and TIM-3 on liver infiltrating donor CD8⁺ T cells. FIG. 34B shows that donor CD8⁺ T cells from BrdU-treated recipients are first shown CD8 versus BrdU; Gated CD8⁺ T cells are also shown in histogram of Annexin V. FIG. 34C shows the yield of liver infiltrating donor CD8⁺ T cells. FIG. 34D shows the serum levels of ALT, AST, and ALB of anti-CD4-treated WT or PD-L1^{-/-} recipients.

FIGS. 35A-35C show that depletion of donor CD4⁺ T cells rendered liver infiltrating donor CD8⁺ T cells susceptible to exhaustion. WT BALB/c recipients were given HCT and anti-CD4 mAb treatment as described in FIG. 11. 21 days after HCT, liver infiltrating donor CD8⁺ T cells were analyzed for exhaustion related markers (CD80, PD-1, and TIM-3), cytokine production, and proliferation, as well as tested for GVHD capacity in adoptive recipients. FIGS. 35A and 35B show that the liver infiltrating donor CD8⁺ T cells were stained for CD80, PD-1, and TIM-3 as well as intracellular IFN-γ and TNF-α. Representative flow cytometry patterns and mean±SE of MFI of CD80, PD-1, and TIM-3 or percentage of IFN-γ⁺TNF-α⁺ cells are shown; N=6 (*p<0.05, **p<0.01, ***p<0.001). FIG. 35C shows the gated donor CD8⁺ T cells in CD8 versus BrdU. Representative flow patterns and Mean±SE of percentage of BrdU⁺ cells are shown. N=6.

FIGS. 36A-36C show that depletion of donor CD4⁺ T cells augmented thymic infiltrating CD8⁺ T cell anergy. Lethally irradiated WT or PD-L1^{-/-} BALB/c recipients received HCT and were treated with IgG or anti-CD4 mAb as described in FIGS. 14, 20 and 33. At day 7 after HCT, total thymocyte yield and thymic infiltrating donor CD8⁺ T cells were analyzed. There were 6 mice in each group combined from 3 replicate experiments (*p<0.05, **p<0.01, ***p<0.001). FIG. 36A shows the yield of total live thymic mononuclear cells (MNCs). FIG. 36B shows CD80, PD-1, GRAIL, and IL7Rα expression on thymus infiltrating H-2K^{b+}TCRβ⁺CD8⁺ donor T cells. Representative patterns and mean±SE of MFI are shown. FIG. 36C shows the percentage and yield of H-2K^{b+}TCRβ⁺CD8⁺ T cells among total live thymic mononuclear cells.

FIGS. 37A and 37B show that an injection of anti-IL-2 mAb after HCT prevented acute GVHD in BALB/c recipients with C57BL/6 transplants. Lethally irradiated BALB/c recipients transplanted with purified CD4⁺ T cells (1 or 2×10⁶) and TCD-BM (2.5×10⁶) from C57BL/6 donors were given i.v. injection of rat-IgG or neutralizing anti-IL2 mAb (500 μg/mouse). Group 1: 1×10⁶ CD4⁺ T cells (every other day from day 0 until day 6), Group 2: 2×10⁶ group (every other day from day 0 until day 21). Recipients were monitored for clinical signs of GVHD, including body weight change, diarrhea, and survival. FIG. 37A shows percentage of body weight change, percentage of recipients without diarrhea, and percentage of surviving recipients in Group 1. n=5 per group. FIG. 37B shows percentage of body weight change, percentage of recipients without diarrhea, and percentage of surviving recipients in Group 2. n=4-6 per group.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

Many factors and pathways may contribute to acute GVHD or chronic GVHD after HCT. Disclosed herein are methods of preventing or treating acute GVHD and chronic GVHD while preserving GVL effects, methods of augmenting expansion of donor CD8+ T cells in lymphoid tissues in vivo after HCT, and methods of augmenting recipient tissue expression of PD-L1 after HCT. GVHD prevention and treatment, as well as in vivo expanding donor CD8+ T cells, can be achieved by temporarily depleting CD4+ T cells using an anti-CD4 agent such as an anti-CD4 antibody or an anti-CD4-meditope-immunotoxin, neutralizing IL-2 using an anti-IL2 antibody, or administering other agents blocking IL-2R. Moreover, other therapeutic agents, such as PD-L1 antibodies and/or IFN-γ can be administered to the subject receiving HCT.

PD-L1 interacts with PD-1 and CD80, and functions as a checkpoint that regulates immune responses in animal models and humans. It is disclosed herein that in allogeneic and xenogeneic murine models of graft-versus-host disease (GVHD), temporary depletion of donor CD4+ T cells immediately after hematopoietic cell transplantation (HCT) effectively prevents GVHD while preserving strong graft-versus-leukemia (GVL) effects. Depletion of donor CD4+ T cells increases serum IFN-γ but reduces IL-2 concentrations, leading to upregulated expression of PD-L1 by recipient GVHD target tissues and by donor CD8+ T cells. In GVHD target tissues, the interactions of PD-L1 with PD-1 on donor CD8+ T cells induced tolerance through anergy, exhaustion and apoptosis of effector T cells, thereby preventing GVHD. In lymphoid tissues, the interactions of PD-L1 with CD80 augment CD8+ T cell expansion and activity against malignant cells in the recipient, without increasing anergy, exhaustion or apoptosis, resulting in strong GVL effects. These results show that the outcome of PD-L1-mediated signaling in CD8+ T cells depends on the presence or absence of CD4+ T cells, the nature of the interacting receptor expressed by CD8+ T cells, and the tissue environment where the signaling occurs.

As detailed in this disclosure, augmenting CD8+ T cells in lymphoid tissues as well as expressing PD-L1 in recipient tissues by administering a therapeutic agent to the recipient has unexpectedly prevented or treated not only acute GVHD but also chronic GVHD. Surprisingly, a single dose of the therapeutic agent is sufficient to prevent or treat acute GVHD and as few as three doses of the therapeutic agent administered within one month are sufficient to prevent or treat chronic GVHD.

The term “recipient,” “host,” “subject,” or “patient” as used herein refers to a subject receiving hematopoietic cell transplantation. These terms may refer to, for example, a subject receiving an administration of donor bone marrow, donor T cells, donor spleen cells, or other donor cells or tissue. In some embodiments, the transplanted cells are derived from an allogeneic donor. The recipient, host, subject, or patient can be an animal, a mammal, or a human.

The term “donor” as used herein refers to a subject from whom the cells or tissue are obtained to be transplanted into a recipient or host. For example, a donor may be a subject from whom bone marrow, T cells, spleen cells, or other cells or tissue to be administered to a recipient or host is derived. The donor or subject can be an animal, a mammal, or a human.

The terms “treat,” “treating,” and “treatment” as used herein with regard to a GVHD condition refer to alleviating the condition partially or entirely, or eliminating, reducing, or slowing the development of one or more symptoms associated with the condition. In some embodiments, the term “treat,” “treating,” or “treatment” means that one or more symptoms of GVHD condition or complications are alleviated in a subject receiving the treatment as disclosed herein comparing to a subject who does not receive such treatment.

The terms “prevent,” “preventing,” and “prevention” as used herein with regard to a GVHD condition refer to preventing the onset of the condition and/or symptoms associated with the condition from occurring, decreasing the likelihood of occurrence or recurrence of the condition, or slowing the progression or development of the condition.

The phrase “an effective amount” or “a therapeutically effective amount” as used herein refers to an amount of a therapeutic agent that produces a desired therapeutic effect. For example, an effective amount of an anti-CD4 antibody may refer to that amount that prevents or treats GVHD, depletes CD4+ T cells, augments CD8+ T cells, or induces tissue expression of PD-L1 in a recipient. The precise effective amount is an amount of the therapeutic agent that will yield the most effective results in terms of efficacy in a given subject. This amount will vary depending upon a variety of factors, including but not limited to the characteristics of the therapeutic agent (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

In one aspect, the disclosure provided herein relates to a method of preventing or treating chronic GVHD after HCT while preserving GVL. The method entails in vivo administering two or more doses of an effective amount of a therapeutic agent to a recipient simultaneously or immediately after HCT to temporarily deplete CD4+ T cells.

The term “simultaneously” as used herein with regards to administration means that the therapeutic agent is administered to the recipient at the same time or nearly at the same time of HCT. For example, the therapeutic agent is considered to be administered “simultaneously” if it is administered via a single combined administration of hematopoietic cells, two or more administrations occurring at the same time, or two or more administrations occurring in succession without extended intervals in between.

When the therapeutic agent is administered immediately before HCT, a first dose of the therapeutic agent can be administered any time up to about 10 days before HCT. When the therapeutic agent is administered immediately after HCT, a first dose of the therapeutic agent can be administered any time up to about 6 weeks after HCT. In some embodiments, a first dose of the therapeutic agent is administered about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3 days, about 4 days, about 5 days, about 6 days, about 7 days, about 8 days, about 9 days, or about 10 days, before HCT. In some embodiments, a first dose of the therapeutic agent is administered simultaneously with HCT. In some embodiments, a first dose of the therapeutic agent is administered about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, or about 6 weeks, after HCT.

When multiple doses of the therapeutic agent are administered, it is within the purview of one of ordinary skill in the art to adjust the administration schedule to optimize the therapeutic effect. For example, one dose can be administered immediately before HCT, followed by additional doses administered during and/or immediately after HCT. In some embodiments, one or more doses of the therapeutic agent can be administered subsequently after the administration of the first dose, e.g., within one month of administration of the first dose. For example, the subsequent doses of the therapeutic agent can be administered in one-week intervals or in two-week intervals.

By administering one or more doses of the therapeutic agent depleting CD4+ T cells immediately after HCT, the donor CD4+ T cells as well as de novo generated CD4+ T cells are completely and temporarily depleted. For example, at least 90%, at least 95%, at least 98%, or at least 99% of the CD4+ T cells are depleted. The CD4+ T cells are depleted for only a short period of time, for less than 10 weeks, for less than 9 weeks, for less than 8 weeks, for less than 7 weeks, for less than 6 weeks, for less than 5 weeks, for less than 4 weeks, for less than 3 weeks, or for about two weeks. In some embodiments, the CD4+ T cells are depleted for at least two weeks.

Any therapeutic agent that effectively depletes CD4+ T cells in vivo for a temporary period of time can be used. In some embodiments, the therapeutic agent is an anti-CD4 antibody, preferably a monoclonal antibody or a humanized antibody. For example, a depleting anti-human CD4 mAb is disclosed in U.S. Pat. No. 8,399,621, the content of which is incorporated herein by reference in its entirety. A functional fragment of an anti-CD4 antibody can be used as long as the fragment effectively depletes CD4+ T cells in vivo. In some embodiments, CD4+ T cells can be depleted by administering to the subject an anti-CD4-meditope-immunotoxin. Such meditopes can be made according to technology known in the art (68). It is within the purview of one of ordinary skill in the art to determine the dose of the therapeutic agent to achieve a desired duration period of depleting CD4+ T cells in vivo.

In some embodiments, a therapeutic agent that effectively neutralizes IL-2 in vivo for a temporary period of time can be used. Such agents include but are not limited to anti-IL-2 antibody, including monoclonal antibodies and/or humanized antibodies, or other agents blocking IL-2R. Certain anti-IL-2 receptor antibodies are known in the art (76, 77). It was reported that IL-2 administration was able to prevent acute GVHD or chronic GVHD (69, 70). Surprisingly, administration of an IL-2 antibody is effective in preventing or treating acute GVHD, as disclosed herein.

In some embodiments, a therapeutic agent includes a PD-L1-Ig. Administration of one or more doses of a therapeutically effective amount of a PD-L1-Ig can also prevent or treat GVHD.

In some embodiments, one or more doses of IFN-γ can be administered to the subject in the absence of CD4+ T cells or at a reduced serum level of IL-2 to help preserve GVL.

In another aspect, the disclosure provided herein relates to a method of preventing or treating acute GVHD after HCT while preserving GVL. The method entails in vivo administering an effective amount of a therapeutic agent to a recipient simultaneously, immediately before, or immediately after HCT to temporarily deplete CD4+ T cells or to temporarily reduce the serum IL-2. Surprisingly, only a single dose of the therapeutic agent is sufficient to prevent or treat acute GVHD. In some embodiments, a single dose of an anti-CD4 antibody is sufficient to prevent or treat acute GVHD. In some embodiments, multiple doses of an anti-IL-2 antibody is administered. For example, an anti-IL-2 antibody can be injected to a subject receiving HCT every other day for up to 30 days to effectively prevent gut GVHD.

In some embodiments, the single dose of the therapeutic agent is administered to the recipient simultaneously with HCT, as described above. Or the single dose of the therapeutical agent is administered immediately before or immediately after HCT, as described above.

In another aspect, the disclosure provided herein relates to a method of augmenting expansion of donor CD8+ T cells in lymphoid tissues in vivo after HCT. The method entails in vivo administering an effective amount of a therapeutic agent to a recipient simultaneously, immediately before, or immediately after HCT to temporarily deplete CD4+ T cells or to temporarily reduce serum IL-2.

In recipient lymphoid tissues, donor CD8+ T cell proliferation is augmented without increasing CD8+ T cell anergy or apoptosis, thereby to achieve strong GVL effects. Surprisingly, in GVHD target tissues, anergy and apoptosis of infiltrating CD8+ T cells are increased in a manner dependent on recipient PD-L1 expression, thereby preventing damage to intestinal Paneth cells and stem cells, hepatocytes, and thymic medullary epithelial cells.

In another aspect, the disclosure provided herein relates to a method of augmenting recipient tissue expression of programmed death-ligand 1 (PD-L1, or B7H1) after HCT. The method entails administering an effective amount of a therapeutic agent to a recipient simultaneously, immediately before, or immediately after HCT to temporarily deplete CD4+ T cells or to temporarily reduce serum IL-2.

Although down-regulation of GVHD by host tissue PD-L1 has been previously reported with the use of PD-L1 deficient recipients (25, 26), this disclosure for the first time demonstrates that host tissue expression of PD-L1 can be achieved by administration of a CD4+ T cells-depleting agent immediately after HCT to effectively prevent both acute and chronic GVHD and preserve strong GVL effects. Depletion of donor CD4+ T cells increases serum IFN-γ concentration and enhances recipient tissue expression of PD-L1 and donor CD8+ T cell expression of the PD-L1 receptors CD80 and PD-1.

As detailed in this disclosure, temporary in vivo depletion of donor CD4+ T cells immediately after transplantation increases donor CD8+ T cell susceptibility to anergy and apoptosis, an effect mediated by expression of PD-L1 in GVHD target tissues. Host expression of PD-L1 has little effect on donor CD8+ T cell expansion in the lymphoid tissues immediately after HCT. Temporary in vivo depletion of donor CD4+ T cells augments donor CD8+ T expansion and allows potent CD8+ T cell-mediated GVL effects in recipient lymphoid tissues. PD-L1 interactions with CD80 and PD-1 mediates donor CD8+ T cell anergy, exhaustion, and apoptosis in different GVHD target tissues in a tissue-specific manner.

With murine models of allogeneic GVHD that reflects characteristic features of acute and chronic GVHD in humans and a murine model of xenogeneic GVHD induced by human PBMC (11, 39, 53), the working examples disclosed herein demonstrate that temporary in vivo depletion of adoptively transferred mature donor CD4+ T and de novo-generated CD4+ T cells immediately after HCT prevents both acute and chronic GVHD while augmenting donor early CD8+ T expansion in lymphoid tissues and preserving strong GVL effects. This outcome does not simply reflect depletion of donor CD4+ T cells that recognize recipient alloantigens, but results from several newly observed mechanisms. Depletion of donor CD4+ T cells leads to increase of serum IFN-γ and decrease of IL-2 concentrations. Depletion of donor CD4+ T cells also leads to expansion of donor CD8+ T cells via T-T and PD-L1/CD80 interactions in lymphoid tissues where they mediate strong GVL effects. At the same time, depletion of donor CD4+ T cells enables host-tissue expression of PD-L1 to induce anergy, exhaustion, and apoptosis of CD8+ T cells infiltrating GVHD target tissues via PD-L1/PD-1 interactions in a tissue-specific manner.

Expression of PD-L1 in recipient tissues can prevent both acute and chronic GVHD after effective depletion of donor CD4+ T cells immediately after HCT, and temporary depletion for only 30-60 days after HCT is sufficient. As demonstrated in the working examples, a single injection of anti-CD4 effectively prevented acute GVHD, but the recipients still developed chronic GVHD with damage in GVHD target tissues, especially in the salivary gland. The working examples further demonstrate that at least three injections were required to effectively prevent chronic GVHD. Three injections of anti-CD4 allowed medullar thymic epithelial cell (mTEC) recovery and restoration of thymic negative selection, but a single injection was not sufficient. It was reported that de novo generated CD4+ T cells immediately after HCT could perpetuate CD8+-mediated damage in the thymus, leading to autoimmune-like chronic GVHD (11). Although a single injection of anti-CD4 prevented acute GVHD and augmented de novo generation of donor-type CD4+ T cells, it did not prevent thymus damage mediated by de novo-generated donor CD4+ T cells immediately after HCT. On the other hand, in the absence of donor CD4+ T cells, donor CD8+ T cells infiltrating thymic tissues were tolerized by host-tissue PD-L1, and thymus damage-mediated by the donor CD8+ T cells was self-limited. Therefore, anti-CD4 treatment has the important effect of temporarily depleting both the injected mature CD4+ T cells and also the CD4+ T cells generated de novo from the marrow early after HCT, thereby allowing sufficient time for mTEC to recover and restore effective thymic negative selection. This time period is proximately 30-60 days after HCT. CD4+ T cells generated from the donor marrow after this time point no longer cause chronic GVHD.

Clinical GVHD prevention is usually associated with reduction of alloreactive T cell expansion and proinflammatory cytokine (i.e. IFN-γ and TNF-α) production. The working examples demonstrate that a single injection of depleting anti-CD4 immediately after HCT effectively prevented acute GVHD, even though the depletion of donor CD4+ T cells led to strikingly increased serum IFN-γ concentrations immediately after transplantation. These results were unexpected since IFN-γ contributes to the pathogenesis of gut GVHD and exacerbates GVHD after PD-1 blockade in recipients transplanted with both donor CD4+ and CD8+ T cells (41, 54). On the other hand, these results are consistent with results reported by Yang et al. (55) who showed that in the absence of donor CD4+ T cells, IFN-γ-deficient donor CD8+ T cells proliferated more vigorously and caused more severe GVHD than WT donor CD8+ T cells.

As shown in the working examples, increased IFN-γ concentrations were associated with enhanced expression of PD-L1 by colon epithelial cells and IFN-γ deficient donor cells was associated with down-regulation of PD-L1 expression. These observations are consistent with results of a previous study showing that in recipients with acute GVHD, upregulation of PD-L1 expression in host tissues requires IFN-γ (29). Although host tissue PD-L1 had little impact on donor CD8+ T cell proliferation or survival in the spleen immediately after HCT, depletion of donor CD4+ T cells led to induction of apoptosis of infiltrating donor CD8+ T cells by PD-L1 in colon tissue and induction of anergy and exhaustion by PD-L1 in liver tissue. The differential effect of PD-L1-mediated signaling on donor CD8+ T cells in the colon and liver was associated with differential expression of PD-1 and CD80 by donor CD8+ T cells in these tissues. The ratio of PD-1 versus CD80 MFI on donor CD8+ T cells was significantly higher in the colon as compared to the liver.

NKT cells, myeloid suppressor cells (MDSCs), and regulatory T cells can suppress GVHD (5, 58) and some of these cells express CD4 and could be depleted by anti-CD4-treatment. However, the working examples demonstrate that depletion of donor CD4+ T cells together with those CD4+ regulatory cells was able to effectively prevent GVHD, suggesting that in the absence of donor CD4+ T cells, tissue protective mechanisms are sufficient to prevent GVHD mediated by CD8+ T cells, and CD4+ regulatory T cells are dispensable.

The working examples demonstrate that small numbers of donor CD4+ T cells in the graft could augment acute GVHD by markedly reducing the apoptosis of CD8+ T cells infiltrating the colon, and this effect was IL-2 dependent. In addition, although sorted CD8+ T cells induced little GVHD, and sorted CD4+ T cells induced severe acute GVHD in PD-L1 sufficient wild-type recipients, sorted CD8+ and CD4+ T cells both induced lethal acute GVHD with similar severity in PD-L1-deficient recipients. These results suggest that CD8+ T cells are more sensitive than CD4+ T cells to host-tissue PD-L1-mediated apoptosis, and CD4+ T cell help immediately after HCT can make donor CD8+ T cells resistant to host-tissue PD-L1-mediated apoptosis. This observation is consistent with a previous report that IL-2 from CD4+ T cells may prevent apoptosis induced by PD-1 signaling in CD8+ T cells that are deficient in IL-2 production (59). These results support that host-tissue expression of PD-L1 could ameliorate GVHD only to a certain degree when whole spleen cells with both CD4+ and CD8+ T cells were transplanted, as indicated by comparing WT and PD-L1−/− recipients (27, 28). As demonstrated in the working examples, increased host tissue expression of PD-L1 induced by higher concentrations of IFN-γ combined with increased sensitivity of donor CD8+ T cells to PD-L1-induced apoptosis in the presence of lower IL-2 concentrations could explain the highly effective prevention of GVHD that was found when donor CD4+ T cells were depleted immediately after transplantation. Depletion of donor CD4+ T cells immediately after HCT not only prevented GVHD, but also enabled donor CD8+ T cell expression of PD-L1 to mediate their own expansion in lymphoid tissues and mediate strong GVL activity that could overcome “GVL-resistant” BC-CML tumor cells. The high concentrations of IFN-γ associated with CD4+ T cell depletion could contribute to the preservation of GVL activity, since Yang et al. (55) showed that in the absence of donor CD4+ T cells, IFN-γ-deficient donor CD8+ T cells had lower GVL activity than WT donor CD8+ T cells.

The working examples demonstrate that anti-CD4-treatment immediately following HCT augments donor CD8+ T cell expansion in the lymphoid tissues, which is dependent on donor CD8+ T expression of both PD-L1 and CD80, and host-tissue expression of PD-L1 has little impact. The lack of impact from host PD-L1 is likely due to relative paucity of host parenchymal cells that express PD-L1 in the lymphoid tissues. The expansion of donor CD8+ T cells in lymphoid tissues most likely results from T-T interaction via PD-L1/CD80, although the possibility that CD8+ T interaction with non-T cells via PD-L1/CD80 cannot be excluded. First, PD-L1 deficiency on donor CD8+ T cells, but not PD-L1 deficiency on donor non-T cells (data not shown) markedly reduced donor CD8+ T cell survival and expansion. Second, CD80 deficiency on donor CD8+ T cells also reduced donor CD8+ T expression of survival gene BCL-XL and increased CD8+ T cell exhaustion. Third, anti-CD4-treatment immediately after HCT upregulated PD-L1 and CD80 expression by donor CD8+ T cells but not by non-T cells (i.e. DCs and myeloid cells). Finally, specific blockade of PD-L1/CD80 interaction by anti-PD-L1 (43H12) markedly reduced donor CD8+ T survival and expansion in the spleen and abolished GVL effects. This result is consistent with previous findings that PD-L1 on CD8+ T cells was required for survival of activated CD8+ T cells (60). In contrast, PD-L1/CD80 interactions augment apoptosis of activated CD4+ T cells (31).

Saha et al showed that PD-L1 deficiency in donor T cells reduced proliferation and survival of donor T cells and delayed GVHD lethality in recipients given both CD4+ and CD8+ donor T cells (30). This observation suggests that even when donor T cells have reduced survival capacity due to lack of expression of PD-L1, host tissue expression of PD-L1 is still unable to tolerize tissue infiltrating T cells to prevent GVHD when both donor CD4+ and CD8+ T cells were present. The working examples show that depletion of donor CD4+ T cells immediately after HCT allowed host PD-L1 to effectively tolerize infiltrating CD8+ T cells in GVHD target tissues and completely prevent acute GVHD. At the same time, PD-L1/CD80 interactions among donor CD8+ T cells in the lymphoid tissues augment CD8+ T cell survival and expansion as well as their GVL activity.

On the other hand, using a non-lethal murine model of GVHD mediated by H-Y antigen-specific transgenic CD8+ T cells, Michonneau et al reported that the transgenic CD8+ T cells were not able to eliminate host-type tumor cells in lymphoid tissues due to enhanced PD-L1/PD-1 interactions between PD-1+ transgenic CD8+ T cells and PD-L1+ CD11c+ DCs and F4/80+ macrophages (52). Similarly, Mueller et al showed that PD-L1 expressed by hematopoietic cells suppressed viral-specific CD8+ cell activation and expansion (61). Although the working examples show that CD11+ DCs and MAC-1/Gr-1+ myeloid cells in the spleen expressed much higher levels of PD-L1 as compared to those in the liver, the high levels of PD-L1 on DCs and myeloid cells did not appear to interfere with the GVL activity of donor CD8+ T cells in the lymphoid tissues of anti-CD4-treated recipients, since different types and dose of tumor cells were all eliminated in anti-CD4-treated recipients.

Several explanations might account for the different impact of PD-L1-expressed by hematopoietic cells on GVL effects in the lymphoid tissues of recipients given H-Y-specific CD8+ T cells as compared to wild-type alloreactive CD8+ T cells. First, PD-L1 expressed by hematopoietic cells mainly control activation and expansion of naïve T cells (61). In anti-CD4-treated recipients, alloreactive CD8+ T cells are activated by recipient APCs that are rapidly eliminated. Therefore, PD-L1 expression by donor hematopoietic-derived APCs does not play an important role on donor T cell activation and expansion. Second, H-Y-specific transgenic CD8+ T cells in male recipients appeared to have very weak alloreactivity as indicated by lack of GVHD mortality even after blockade of PD-1. Their alloreactivity was easily controlled by PD-L1/PD-1 interactions between CD8+ T cells and DCs and macrophages in the lymphoid tissues. In contrast, the alloreactivity of wild-type alloreactive CD8+ T cells is much stronger, as indicated by their ability to cause rapidly lethal GVHD in PD-L1−/− recipients. Their alloreactivity cannot be controlled by PD-L1/PD-1 interactions between CD8+ T and DCs and macrophage. Third, H-Y-specific transgenic CD8+ T cells might not express PD-L1, or PD-L1 might not play a role in their survival and expansion, unlike wild-type alloreactive T cells (30).

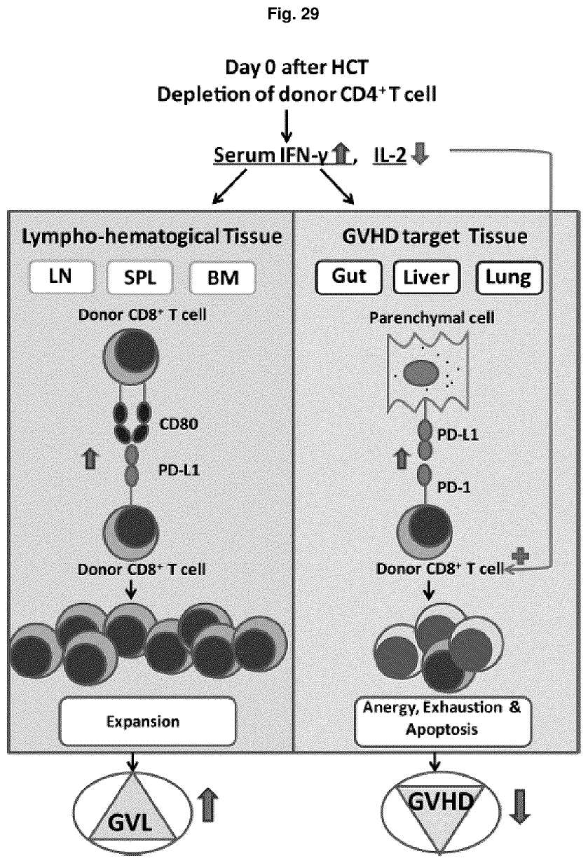

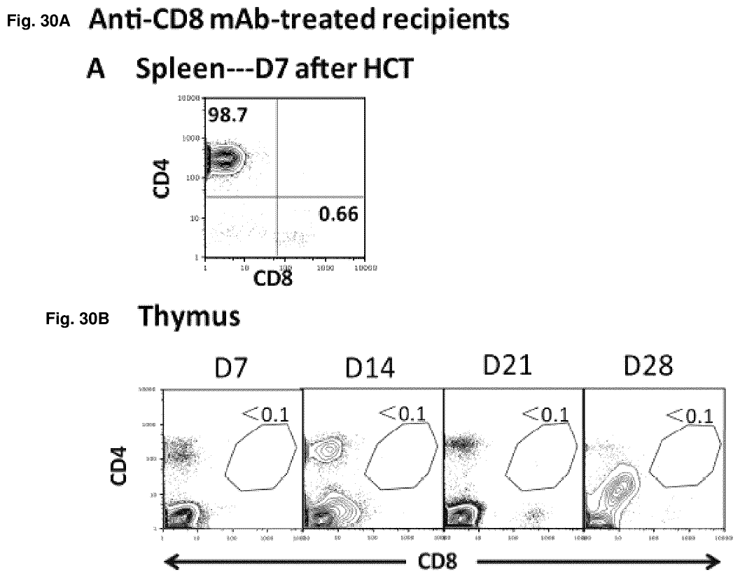

As depicted in FIG. 29, depletion of donor CD4+ T cells immediately after HCT increases serum IFN-γ but decreases IL-2 concentrations. Increase of IFN-γ augments expression of PD-L1 by donor CD8+ T cells and host tissues, while increasing expression of PD-1 and CD80 by donor CD8+ T cells. Donor CD8+ T cells express higher levels of PD-L1 and CD80 but lower level of PD-1 in the spleen, promoting PD-L1/CD80 interactions among donor CD8+ T cells. In contrast, donor CD8+ T cells express higher level of PD-1 and lower levels of PD-L1 and CD80 in GVHD target tissues, promoting host tissue PD-L1 interaction with PD-1 on donor CD8+ T cells. CD8+ T cells are defective in IL-2 production, and in the absence of IL-2 help from CD4+ T cells, donor CD8+ T cells may become more sensitive to the tolerizing effects of PD-L1/PD-1 signaling. Donor CD8+ T-T and PD-L1/CD80 interactions augment donor CD8+ T survival and expansion in lymphoid tissues, resulting in strong GVL effects. Dominant host-PD-L1 interaction with PD-1 on CD8+ T cells mediates donor CD8+ T cell anergy, exhaustion and apoptosis in GVHD target tissues, thereby preventing GVHD.

The working examples support that sorted donor CD8+ T cells facilitate engraftment and mediate GVL effect without causing GVHD (2, 7). The results also demonstrate that ex vivo depletion of donor CD4+ T cells did not effectively prevent GVHD in a previous human trial (62) probably because very small numbers of donor CD4+ T cells in the graft could have expanded after HCT, and they could have worked together with donor CD4+ T cells generated from the marrow progenitors immediately after HCT to help donor CD8+ T cells resist host-tissue PD-L1 mediated apoptosis or other tolerance mechanisms. The results also indicate that temporary in vivo depletion of donor CD4+ T cells immediately after HCT can be a novel approach to prevent GVHD while preserving strong GVL effects. Temporary in vivo depletion of donor CD4+ T cells for a period of approximately 30-60 days after HCT may not only allow GVHD target tissues to tolerize infiltrating donor CD8+ T cells while preserving GVL effects in the lymphoid tissues, but may also allow regeneration of medullary thymic epithelia cells and restoration of thymic negative selection for durable prevention of chronic GVHD.

Permanent depletion of de novo-generated CD4+ T cells is not required for mTEC recovery. The results in the working examples indicate that recovery of mTECs takes time. Autoreactive CD4+ T cells can be generated de novo immediately after HCT before mTEC have adequately recovered, but as time goes on, the mTEC percentage gradually increases, and negative selection is gradually restored. Based on the results disclosed herein, CD4+ T cells generated de novo beyond ˜45 days after HCT no longer cause autoimmunity or chronic GVHD. Therefore, depletion of de novo-generated autoreactive CD4+ T cells immediately after HCT allows time for mTEC recovery and restoration of negative selection in the thymus. The methods disclosed herein should not cause long-term CD4+ T cell deficiency in young recipients with adequate thymic function, although CD4+ T cell reconstitution may be delayed in older recipients.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLES

### Materials and Methods

Induction and scoring of acute GVHD and chronic GVHD, in vivo bioluminescent imaging, in vivo BrdU-labeling of proliferating T cells, TUNEL staining, tissue cell isolation, intracellular staining of cytokines, antibodies, flow cytometry analysis and sorting, histopathology, histoimmunofluorescent staining, and real-time PCR have been described in previous publications (11, 27, 29, 31, 63) and detailed below.

Data are displayed as mean±SEM. Bodyweight, diarrhea, cutaneous damage scoring, GVHD and survival in different groups were compared by using the rank sum test or log-rank test. Comparison of two means was analyzed using an unpaired two-tailed Student t-test (Prism, version 6.0; GraphPad Software) (*p<0.05, **p<0.01, ***p<0.001, ****p<0.0005).

Mice: C57BL/6 (H-2b) and BALB/c (H-2d) mice were purchased from the National Cancer Institute animal production program (Frederick, MD). A/J mice (H-2a) were purchased from the Jackson Laboratory (JAX). PD-L1−/− BALB/c breeders were provided by Dr. Lieping Chen (Yale University). PD-L1−/− C57BL/6 breeders, spleen and bone marrow cells were provided by Dr. Haidong Dong (Mayo Clinic). Congenic CD45.1+ C57BL/6 mice, CD80−/− C57BL/6 breeders and IFN-γ−/− C57BL/6 breeders were purchased from JAX Lab. Rag2−/− BALB/c mice were purchased from Taconic Farms (Germantown, NY). NSG mice were provided by the Animal Tumor Model Core (City of Hope). All mice were maintained in a pathogen-free room in the City of Hope Animal Resource Center. All animal protocols were approved by COH Institutional Animal care and use committee (IACUC).

Induction and assessment of GVHD: BALB/c recipients were exposed to 850 cGy total body irradiation (TBI) with the use of a [137Cs] source 8-10 hours before HCT, and then injected intravenously (i.v.) with C57BL/6 donor spleen cells (2.5×106 or 5.0×106) and T cell-depleted BM (TCD-BM) (2.5×106). C57BL/6 recipients were exposed to 1100 cGy TBI and then injected i.v. with A/J donor spleen cells (10×106, 20×106 or 40×106) or CD8+ TCD spleen and BM cells (10×106). NSG recipients were injected i.p. with human PBMC (20×106) from healthy donors. For secondary transplantation, Rag2−/− BALB/c mice were exposed to 200 cGy TBI 24 h before HCT and were injected i.v with sorted CD8+ T cells (1×106) from the liver of anti-CD4 or rat-IgG-treated primary recipients together with primary recipient strain TCD-BM (5×106). T cell depletion from the bone marrow was accomplished by using biotin-conjugated anti-CD4 and anti-CD8 mAbs, and streptavidin Microbeads (Miltenyi Biotec, Germany), followed by passage through an autoMACS Pro cell sorter (Miltenyi Biotec, Germany). Enrichment of Thy1.2+ cells from spleen was accomplished by using mouse anti-CD90.2 microbeads (Miltenyi Biotec, Germany). The purity of enrichment was >98%, whereas the purity of depletion was >99%. The assessment and scoring of clinical acute signs of GVHD and clinical cutaneous GVHD has been described previously (1, 2).

Isolations of cells from GVHD target tissues: Liver samples were mashed through a 70 μm cell strainer, and MNC were isolated from the cell suspensions with Lymphocyte M. Digestion buffer [RPMI containing 5% fetal bovine, 10 mM HEPES, 10 U heparin, collagenase D (1 mg/ml), and DNase I (1000 U/ml)] was carefully injected into lung lobes, and specimens were incubated at 37° C. for 45 min. After a second cycle of digestion, lung tissue were mashed through a 70 μm cell strainer, and MNC were isolated from cell suspensions with Lymphocyte M. Colon specimens were washed in PBS, cut into 0.5 mm pieces and suspended in PBS containing 1% Bovine serum and 0.002M EDTA, vortexed for 10 min., passed through 70 μm strainer and glass wool, and centrifuged for 5 min at 2000 rpm to isolate epithelial cells and lymphocyte.

Antibodies, FACS analysis and FACS sorting: Purified depleting anti-mouse CD4 mAb (GK1.5), blocking anti-mouse PD-L1 (10F.9G2), neutralizing anti-IL-2 (JES6-1A12), and CD8 (53-6.72) for in vivo treatment were purchased from Bio X Cell (West Lebanon, NH). Depleting anti-human CD4 mAb (IT1208) for in vivo treatment was provided by Dr. Ito at IDAC Theranostics. H-2Kb (AF6-88.5), α4β7 (DATK32), Ly51 (6C3) and FITC Annexin V were purchased from BD Pharmingen (San Diego, CA). mAbs to TCRβ (H57-597), H-2Kb (AF6-88.5), CD3 (UCHT1), CD4 (RM4-5), CD8a (SK1), CD8a (53-6.7), CD45 (30-F11), CD11b (M1/70), CD11c (N418), Gr-1 (RB6-8C5), B7H1 (H1M5), PD-1 (RMP1-30), CD44 (IM7), CD62L (MEL-14), EpCAM (G8.8), FASL (MFL3), IL7Rα (A7R34), TIM3 (RMT3-23), IFN-γ (XMG1.2), EOMES (Dan 11 mag) and Foxp3 (FJK-16s) were purchased from eBioscience (San Diego, CA). mAbs to CCR9 (Clone 242503) and IL-22R (Clone 496514) were purchased from R&D Systems (Minneapolis, MN). Anti-CXCR3 mAb and anti-T-bet (4B10) were purchased from Biolegend (San Diego, CA). Polyclonal Rabbit Anti-Human Lysozyme EC 3.2.1.17 was purchased from DAKO (Carpinteria, CA). Anti-RNF128:FITC (GRAIL) mAb (ARP43311_T100) were purchased AVIVA SYSTEMS BIOLOGY (San Diego, CA). Anti-Cytokeratin mAb was purchased from Sigma-Aldrich (Louis, MO). mAb to Ulex europaeus agglutinin 1 (UEA-1) was purchased from Vector Laboratories (Burlingame, CA). Flow cytometry analyses were performed with a CyAn Immunocytometry system (DAKO Cytomation, Fort Collins, CO) and BD LSRFortessa (Franklin Lakes, NJ), the resulting data were analyzed with FlowJo software (Tree Star, Ashland, OR). T cell sorting was performed with a BD FACS Aria SORP sorter at the City of Hope FACS facility. The sorted cells were used for transplantation and real-time RT-PCR.

GVHD target tissue cell isolation: Mononuclear cells (MNCs) from lung, liver and gut were processed and collected as previously described (29). Thymic epithelial cell isolation was performed as previously described (11). In brief, the thymus was cut into small pieces and placed in RPMI 1640 media with collagenase D and DNAse I. Thymic fragments were rapidly mixed through the aperture of a 1000-ml pipette tip and incubated in a 37° C. water bath to digest the thymus and release epithelial cells from the extracellular matrix. Cell suspension was harvested every 15 min, and the process was repeated twice. The harvested cells were incubated with anti-CD45 microbeads, followed by passing through an MACS separation column (Miltenyi Biotec), the negative population containing CD45− mTEC cells were kept for the subsequent flow cytometry analysis. The gut epithelial cell isolation was performed according to a previous report (71). Briefly, colons were washed in PBS and chopped into 0.5 cm pieces. Colon tissue was incubated in 5 mM EDTA and 1 mM DTT with PBS for 30 min at 37° C. while shaking at 200 rpm. Samples were filtered in a 70-μm strainer, centrifuged for 15 min at 1700 rpm layered over 30% Percoll to isolate epithelial cells which were then used for FACS analysis.

In vivo BrdU labeling and annexin V staining: Day 7 or Day 10 after HCT, T cell proliferation was measured with a single intraperitoneal (i.p) injection of BrdU (2.5 mg/mouse, 100 mg/g) 3 hours before tissue harvesting. Day 21 after HCT, T cell proliferation was measured by three i.p. injections every 24 hours with BrdU (2.5 mg/mouse, 100 mg/g) beginning 72 hours before tissue harvesting. Analysis of donor CD8+T cells for BrdU incorporation was performed according to the manufacturer's instructions (BD Pharmingen). For Annexin V staining, the percentage of Annexin V+ cells among donor CD8+ T cells was assessed by flow cytometry according to the manufacturer's instructions (eBioscience, San Diego, CA).

Real-time RT-PCR: Real-time RT-PCR analysis of mRNA for CCL25, CXCL9, CXCL10, CXCL11 was performed as described in the previous publication (1, 6). Primers used are as follows:

CCL25:
Forward:
(SEQ ID NO: 1)
5′-TTACCAGCACAGGATCAAATGG,
Reverse:
(SEQ ID NO: 2)
5′-CGGAAGTAGAATCTCACAGCAC-3′;
CXCL9:
Forward:
(SEQ ID NO: 3)
5′-TCCTTTTGGGCATCATCTTC-3′,
Reverse:
(SEQ ID NO: 4)
5′-TTCCCCCTCTTTTGCTTTTT-3′;
CXCL10:
Forward:
(SEQ ID NO: 5)
5′-CGATGACGGGCCAGTGAGAATG-3′,
Reverse:
(SEQ ID NO: 6)
5′-TCAACACGTGGGCAGGATAGGCT-3′;
CXCL11:
Forward:
(SEQ ID NO: 7)
5′-AGTAACGGCTGCGACAAAGT-3′,
Reverse:
(SEQ ID NO: 8)
5′-GCATGTTCCAAGACAGCAGA-3′;
GAPDH:
Forward:
(SEQ ID NO: 9)
5′-TCACCACCATGGAGAAGGC-3′,
Reverse:
(SEQ ID NO: 10)
5′-GCTAAGCAGTTGGTGGTGCA-3′.

Relative expression levels of genes were normalized within each sample to the house keeping gene GAPDH.

Measurement of cytokines and liver function in serum: Cytokines in serum were measured by enzyme-linked immune sorbent assay (ELISA). The ELISA kits for IFN-γ, TNF-α and IL-2 were purchased from R&D Systems (Minneapolis, MN). ELISA kit for mouse IL-27 was purchased from Biolegend (San Diego, Ca). Measurements of liver function (AST, ALT and ALB) were performed by the Charles River Clinical Pathology Laboratory (Wilmington, MA). Serum AST levels during GVL experiments was measured with Aspartate Aminotransferase activity assay kit purchased from abcam (Cambridge, MA).

Histopathology: Tissue specimens were fixed in formalin before embedding in paraffin blocks, sectioned and stained with H&E. Slides were examined at 200× or 400× magnification and visualized with an Olympus and a Pixera (600CL) cooled charge-coupled device camera (Pixera, Los Gatos, CA). Tissue damage was blindly assessed on a scoring system, as described previously (1, 2). In brief, skin GVHD was scored by tissue damage in the epidermis and dermis and by loss of subcutaneous fat; the maximum score is 9. Salivary GVHD was scored by mononuclear cell infiltration and follicular destruction; the maximum score is 8. Liver GVHD was scored by the severity of lymphocytic infiltrate, number of involved tracts and severity of liver cell necrosis; the maximum score is 9. Lung GVHD was scored by periluminal infiltrates, pneumonitis, and the severity of lung tissues damage; the maximum score is 9. Gut GVHD was scored by mononuclear cell infiltration and morphological aberrations (e.g. hyperplasia and crypt loss), with a maximum score of 8.

Histoimmunofluorescent staining of intestinal Paneth cells and epithelial cells as well as thymic epithelial cells: Small intestine and colon tissues were harvested, formalin-fixed and paraffin embedded. Small intestines were stained with rat-anti-mouse IL-22Rα antibody (R&D Systems) and polyclonal rabbit anti-human lysozyme (Dakocytomation). Colon tissues were stained with anti-cytokeratin-Pan (Sigma). Frozen thymic tissues were put in PFA over night at 4° C., then transferred to sucrose over night at 4° C. Forty-eight hours later, the samples were embedded in OCT gel, frozen on dry ice and stored at −80° C. Thymus were stained with anti-UEA-1 (Vector lab) for medulla epithelial cells and anti-Cytokeratin 8 (DSHB) for cortical epithelial cells.

TUNEL assay of hepatocyte apoptosis: Paraffin sections were stained with DAPI and TUNEL according to the manufacturer's instructions (Roche, Indianapolis, IN) and imaged with the use of an Olympus IX81 Automated Inverted Microscope. Images were taken with a 400× objective and analyzed using Image-Pro Premier.

Bioluminescent imaging: Mice were injected with luciferase+ BCL1 cells (BCL1/Luc+) i.p. and monitored for expansion of those cells using bioluminescent imaging. In vivo imaging of tumor growth has been previously described (7). Briefly, mice were injected with 200 μl firefly luciferin i.p. (Caliper Life Sciences, Hopkinton, MA), anesthetized, and imaged by using an IVIS100 (Xenogen) and AmiX (Spectral) imaging system. Data were analyzed using Igor Pro 4.09A software purchased from WaveMetrics (Lake Oswego, OR) and Amiview software purchased from Spectral Instruments Imaging (New York, NY).

Production of mouse B7H1-Fc: B7H1-Fc-expressing plasmid was a kind gift from Dr. Lieping Chen (Yale University School of Medicine). The DNA plasmid contained the coding sequence for the murine B7H1 extracellular domain that was fused with the CH2-CH3 region of human IgG1 heavy chain. B7H1-Fc fusion protein was expressed transiently in Chinese Hamster Ovary Suspension (CHO-S) cell line using Thermo Fisher Freestyle CHO expression system as manufacture protocol. The supernatant of the transiently transfected CHO-S was collected after 7 days and passed through the protein G agarose beads (GenScript) packed column that had been equilibrated in 1×PBS pH. 7.4. B7H1-Fc bound protein was washed with 1×PBS pH 7.4, eluted with 0.1M Glycine pH 2.5, dialyzed in 1×PBS pH 7.4 and concentrated into 1.0 mg/ml aliquots before freezing in −80° C. until further use.

### Example 1: Effects of Depletion of Donor CD4+ T Cells on GVHD Prevention and GVL Preservation

This example demonstrates that temporary depletion of donor CD4+ T cells immediately after HCT preserves strong GVL effects, while effectively preventing both acute and chronic GVHD in multiple models.

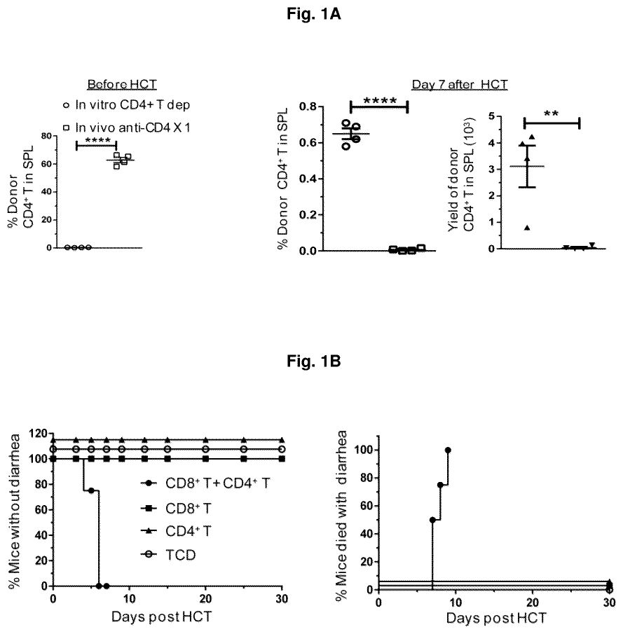

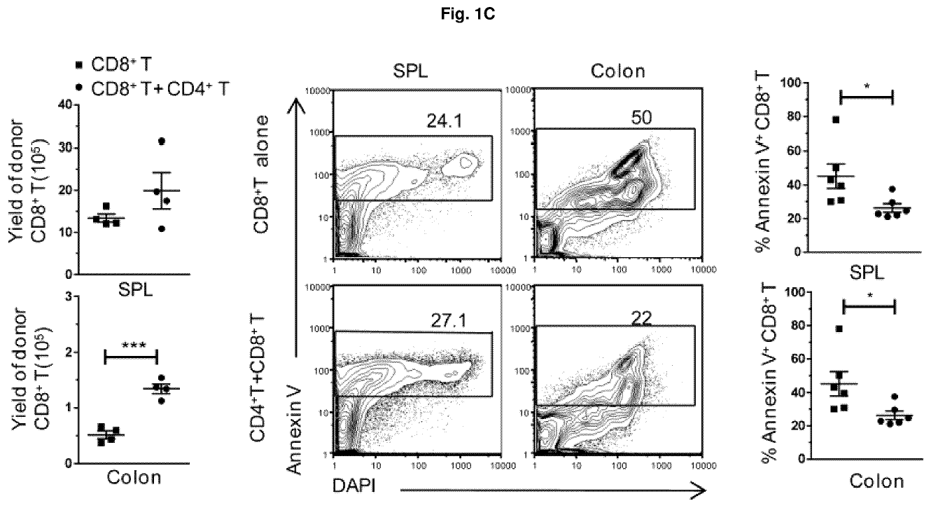

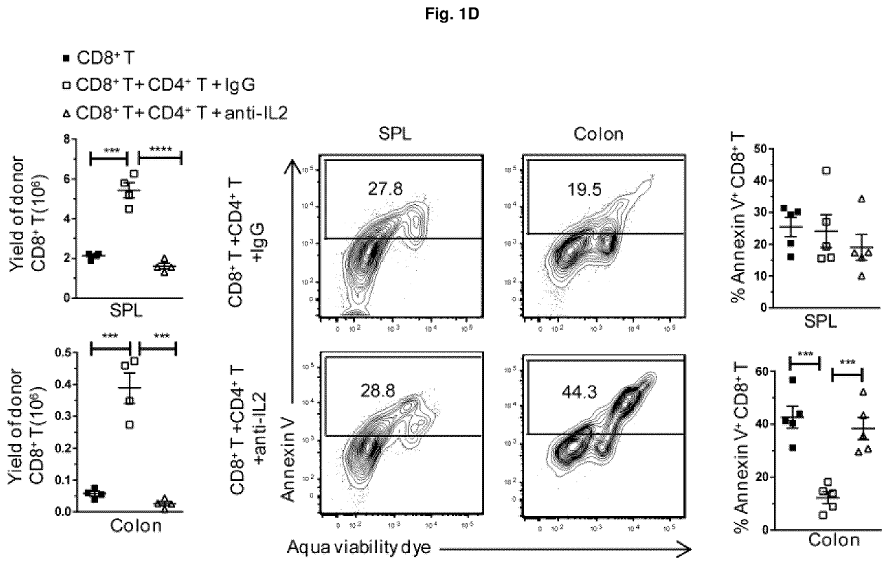

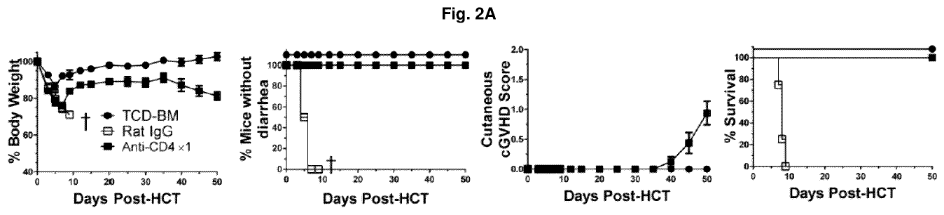

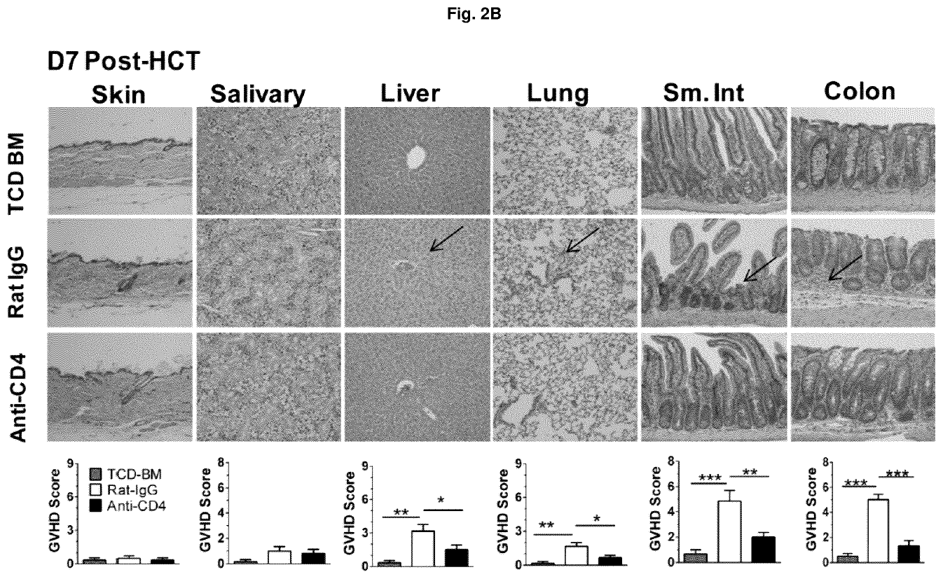

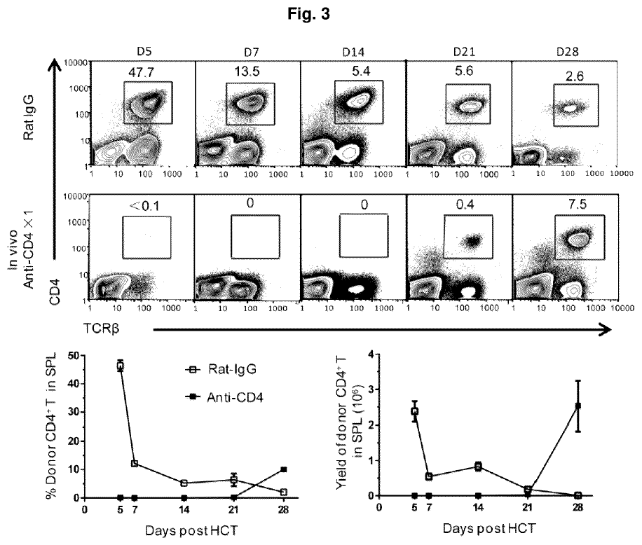

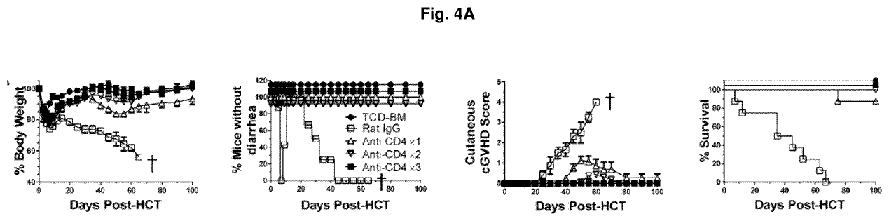

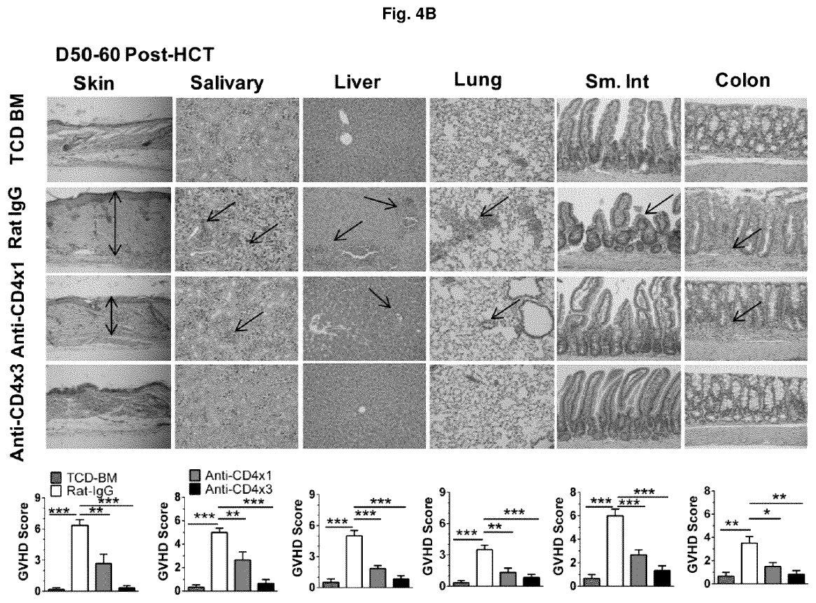

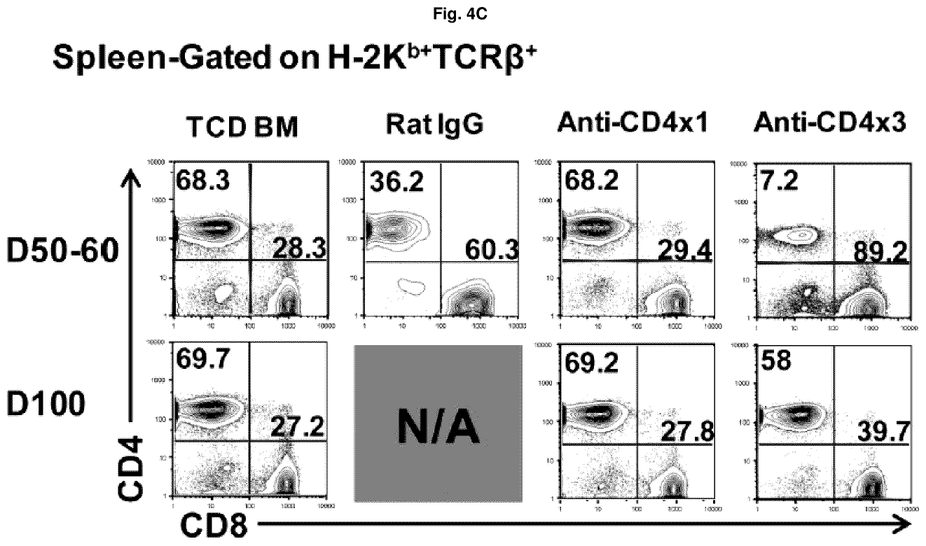

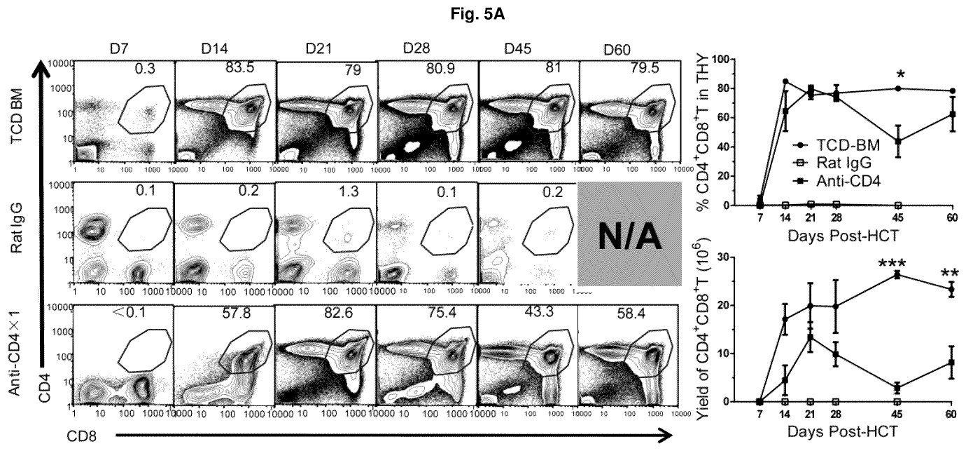

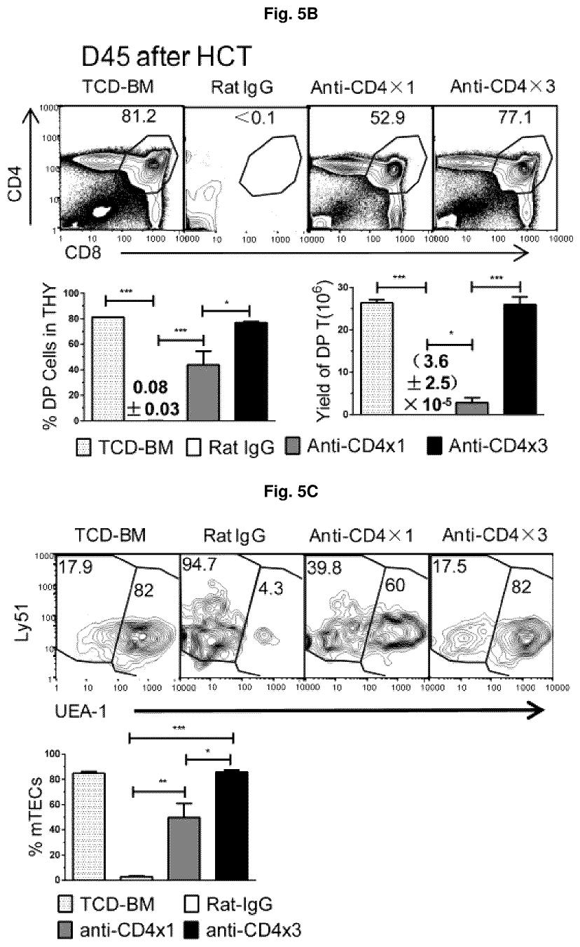

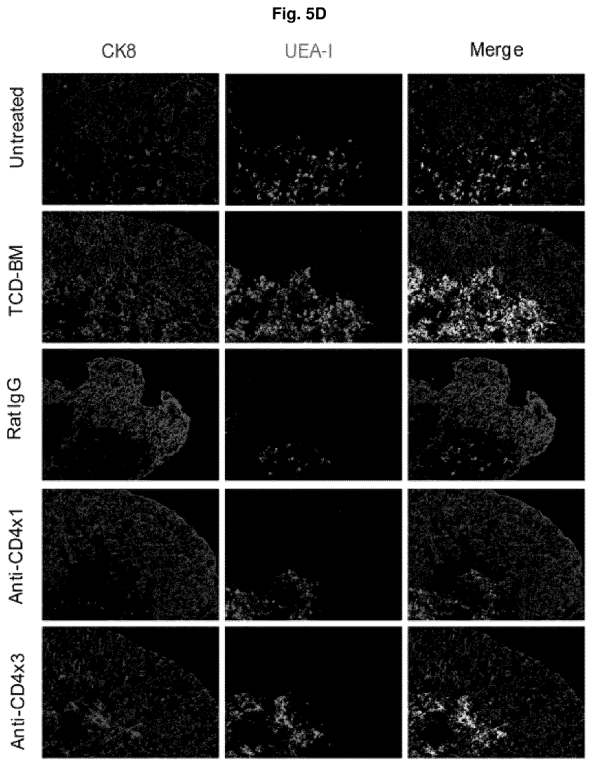

A previous study showed that sorted CD8+ T cells from C57BL/6 donors did not induce acute GVHD but they induced chronic GVHD in lethally irradiated BALB/c recipients, as indicated by histopathology in salivary glands, a prototypic target organ of chronic GVHD. Depletion of CD4+ T cells by treatment with anti-CD4 mAb on days 15 and 30 prevented the development of chronic GVHD, as indicated by prevention of tissue damage in all GVHD target tissues, especially in the salivary gland (11). It is disclosed herein that 1) in vivo administration of anti-CD4 mAb on the day of HCT was more effective in depleting donor CD4+ T cells as compared to ex vivo depletion of CD4+ T cells, as judged by percentage and yield of donor CD4+ T cells in the spleen of recipients at 7 days after HCT (FIG. 1A). 2) Although low-dose of CD4+ T (0.075×106) or CD8+ T (1×106) alone induced no sign of diarrhea, addition of the small numbers of CD4+ T cells to the CD8+ T cell graft induced severe diarrhea and death of all recipients (FIG. 1B). The addition of the small numbers of donor CD4+ T cells markedly reduced the apoptosis of colon tissue infiltrating CD8+ T cells, resulting in marked expansion of donor CD8+ T cells in the colon tissue (FIG. 1C), and this effect was IL-2 dependent (FIG. 1D). 3) A single injection of anti-CD4 mAb immediately following HCT effectively prevented acute GVHD but did not prevent chronic GVHD, which was associated with reconstitution of donor CD4+ T cells beginning by day 21 after HCT (FIG. 2 & FIG. 3). 4) Three injections of anti-CD4 on days 0, 14, 28 effectively prevented both acute and chronic GVHD, and recovery of donor CD4+ T cells thereafter no longer caused chronic GVHD (FIGS. 4A-4C). Prevention of chronic GVHD was indicated by the absence of tissue damage in GVHD target tissues at ˜60 days after HCT (FIG. 4B). In addition, three injections but not one injection of anti-CD4 led to recovery of medullary thymic epithelial cells (mTEC) (FIGS. 5A-5D). Therefore, GVHD, especially chronic GVHD, is more effectively prevented by temporary in vivo depletion of donor CD4+ T cells immediately after HCT than by ex vivo depletion of donor CD4+ T cells.

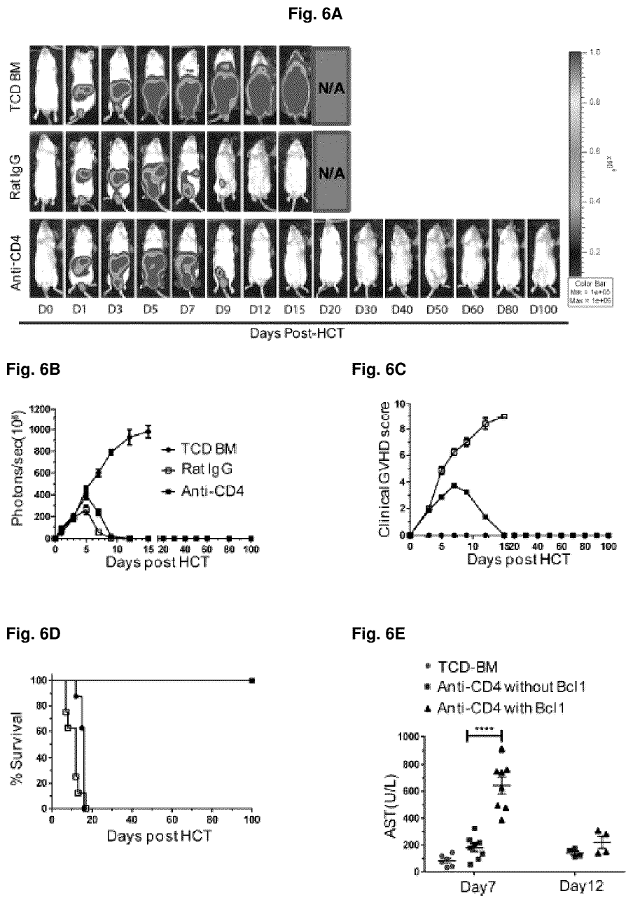

The impact of temporary in vivo CD4+ T cell depletion on GVL effects against BCL1 tumor cells was evaluated (35, 36). TBI-conditioned BALB/c recipients were injected with luciferase-transfected BCL1 cells (BCL1/Luc, 5×106/mouse) together with TCD-BM (2.5×106) alone or TCD-BM+spleen cells (5×106). Recipients given spleen cells were treated with anti-CD4 mAb or control rat IgG on days 0, 14, and 28 days after HCT. BCL1/Luc tumor cell-bearing recipients transplanted with TCD-BM alone all died with progressive tumor growth by 20 days after HCT (FIGS. 6A-6D). Recipients transplanted with BM and spleen cells and treated with control rat IgG eliminated BCL1/Luc tumor cells by 12 days after HCT, but all died with acute GVHD and severe diarrhea by 20 days after HCT. In contrast, recipients treated with anti-CD4 mAb eliminated the tumor cells by 12 days after HCT, and all recipients survived for more than 100 days with little signs of GVHD (p<0.001, FIGS. 6A-6D). These results indicate that in vivo depletion of donor CD4+ T cells immediately after HCT preserves GVL effects while preventing GVHD.

Depletion of donor CD8+ T cells by anti-CD8 (FIG. 30A) did not protect thymus or allow regeneration of DP thymocytes (FIG. 30B).

Leukemia and lymphoma cells also infiltrates liver tissues. In vivo BLI indicated that tumor load started to decrease by day 7 and disappeared by day 12 after HCT (FIGS. 6A-6B). In addition, as compared to recipients given TCD-BM alone, anti-CD4-treated recipients showed mild and transient signs of acute GVHD, peaking at day 7 (FIG. 6C). Thus, it was tested whether CD8+ T cell-mediated GVL activity was associated with hepatocyte damage. The serum aspartate aminotransferase (AST) concentrations in anti-CD4-treated recipients with or without the presence of BCL1 cells were compared. On day 7 after HCT, anti-CD4-treated recipients with BCL1 cells had significantly elevated serum AST concentrations as compared to recipients without BCL1 (P<0.01, FIG. 6E). By day 12, AST concentrations returned to normal, and no difference was observed among anti-CD4-treated recipients with or without BCL1 inoculation and recipients given TCD-BM alone. These results indicate that GVL effect of CD8+ T cells is associated with mild hepatocyte damage, but this damage is self-limited and disappears after tumor cell eradication.

The GVL capacity of this regimen was tested by using “GVL-resistant” blast crisis-chronic myeloid leukemia (BC-CML) in C57BL/6 background. Murine BC-CML cells obtained from W. Shlomchik were generated by retroviral transfer of bcr-abl and NUP98/HOXA9 fusion cDNAs. Like human BC-CML, murine BC-CML was relatively GVL resistant. At certain cell doses, allogeneic CD8+ T cells were not able to rescue recipients inoculated with BC-CML cells, although identical numbers of CD8+ T cells rescued almost all recipients inoculated with same number of chronic-phase chronic myelogenous leukemia (CP-CML) cells (37).

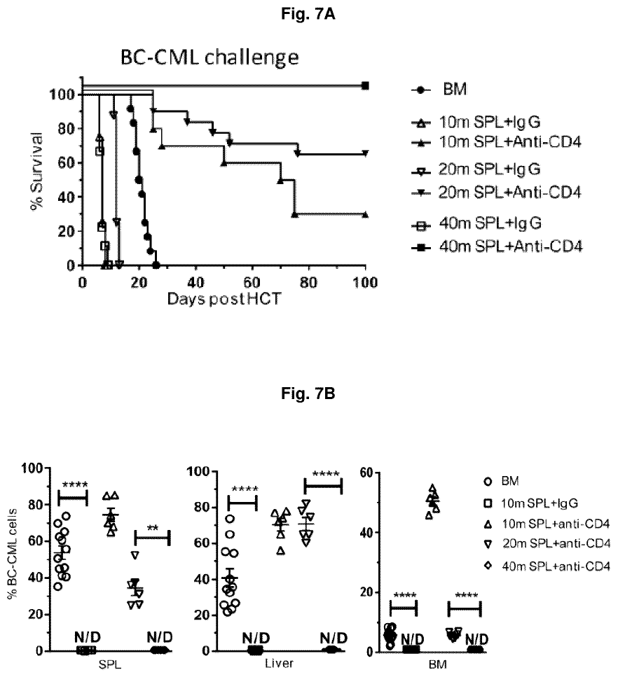

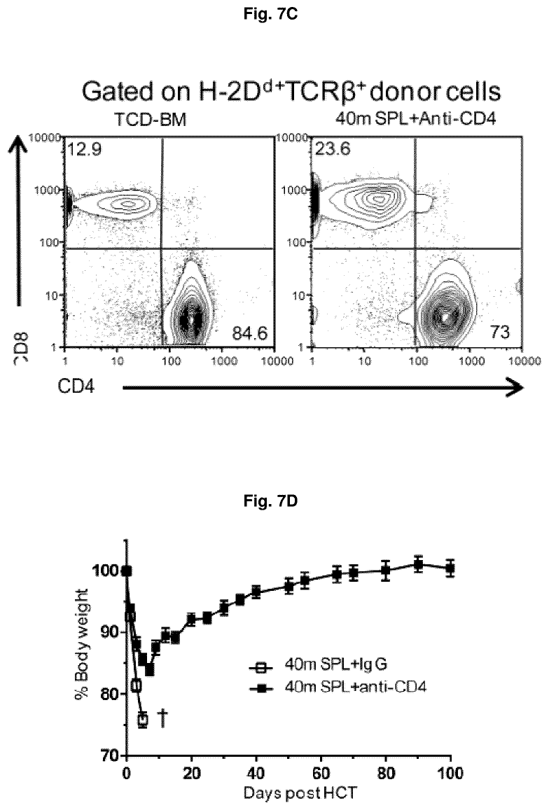

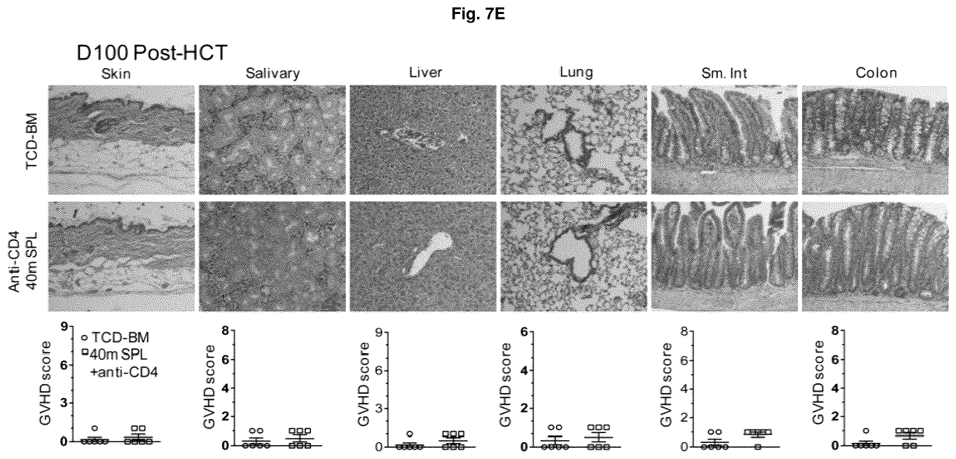

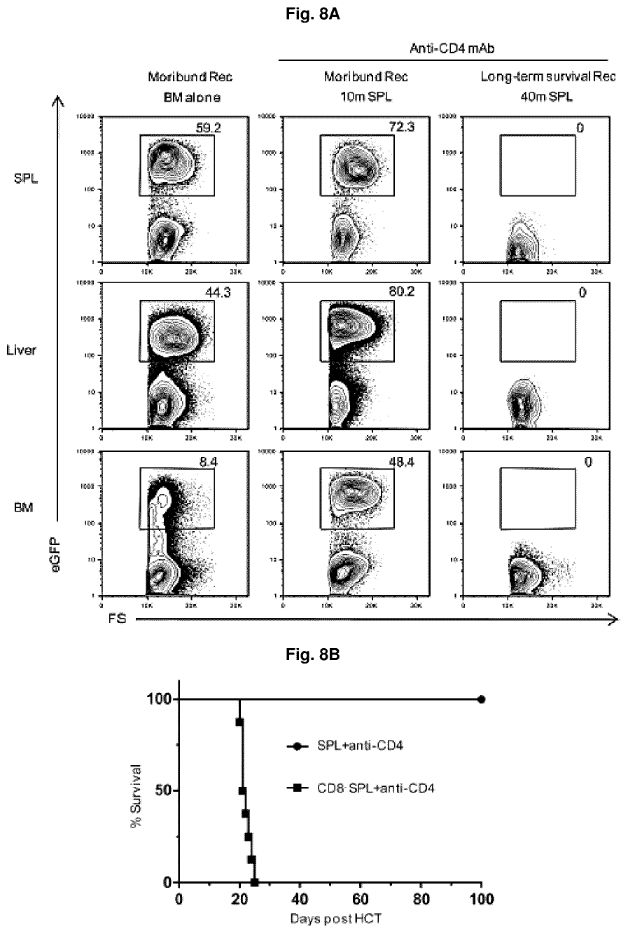

A/J BM (10×106) and spleen cells (10×106) were transplanted into lethally irradiated (1100 cGy) C57BL/6 recipients (38). The recipients were challenged with an intravenous injection of BC-CML (20×103 cells/mouse) at the time of HCT (37). The tumor cells killed all (12/12) GVHD-free recipients given TCD-BM alone within 30 days, and moribund mice had high percentages of BC-CML cells in the spleen, liver and bone marrow (FIGS. 7A, 7B, and 8A). In contrast, tumor cells were eliminated in IgG-treated GVHD-recipients, although they all (8/8) died due to GVHD within 15 days after HCT (FIGS. 7A & 7B). The anti-CD4-treated, GVHD-free recipients given 10×106 donor spleen cells had significantly prolonged survival, as compared with TCD-BM recipients (P<0.01, FIG. 7A), but by day 100 after HCT, 70% (7/10) of the recipients died with progressive tumor growth (FIGS. 7A, 7B, and 8A). The three recipients surviving more than 100 days after HCT had no detectable tumor cells. Thus, BC-CML cells appear to be partially resistant to GVL effects in anti-CD4-treated recipients given 10×106 spleen cells.

In further experiments, donor spleen cells were increased to 20 and 40×106 and the anti-CD4 treatment was extended to day 60 after HCT. 37.5% (6/16) recipients given 20×106 donor spleen cells died with progressive tumor growth, 62.5% (10/16) survived for more than 100 days without detectable tumor cells (FIGS. 7A & 7B). All (12/12) recipients given 40×106 donor spleen cells survived for more than 100 days without detectable tumor cells in the spleen, liver or BM (FIGS. 7A, 7B, and 8A). The anti-CD4-treated recipients given 40×106 donor spleen cells showed recovery of CD4+ T cells to the level similar to TCD-BM recipients by 100 days after HCT (FIG. 7C). They showed no clinical evidence of GVHD. Body weight increased progressively, and histological evaluation showed no tissue damage at day 100, similar to results in TCD-BM control (FIGS. 7D and 7E). The anti-tumor effect was donor CD8+ T cell-dependent, because injection of CD8+ T-depleted spleen cells (40×106) abolished GVL effects in anti-CD4-treated GVHD-free recipients, and all mice (8/8) died with progressive tumor growth by ˜25 days after HCT (FIG. 8B). Taken together, these results show that temporary in vivo depletion of CD4+ T cells allows donor T cells to eliminate “GVL-resistant” BC-CML leukemia cells while effectively preventing GVHD.

Whether administration of depleting anti-human CD4 mAb could prevent GVHD and preserve GVL effects in a xenogeneic model of GVHD was tested (39). NSG mice without or with human B cell lymphoma Raji cells (1×106/mouse) were used for GVHD or GVL experiments. Healthy human PBMC (20×106) were injected i.p. into mice that were then treated with depleting anti-human CD4 (clone IT1208, 200 μg/mouse) or control IgG twice weekly for 4 weeks (40). 4 human PBMC donors were tested. For each donor, 16 NSG mice were used, 8 for GVHD experiments and 8 for GVL experiments. Within each experiment, 4 recipients were treated with control IgG and 4 were treated with anti-CD4.

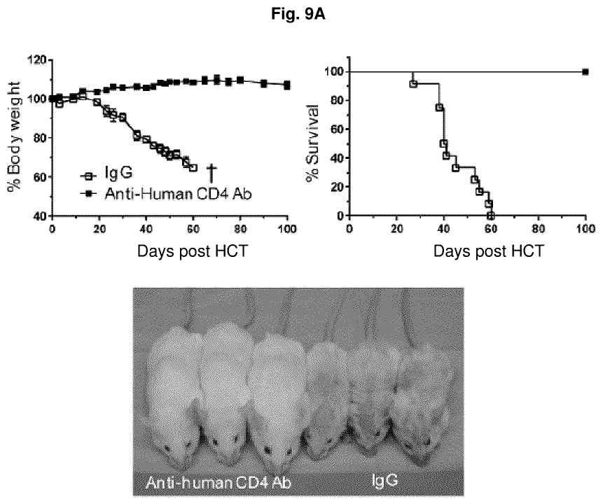

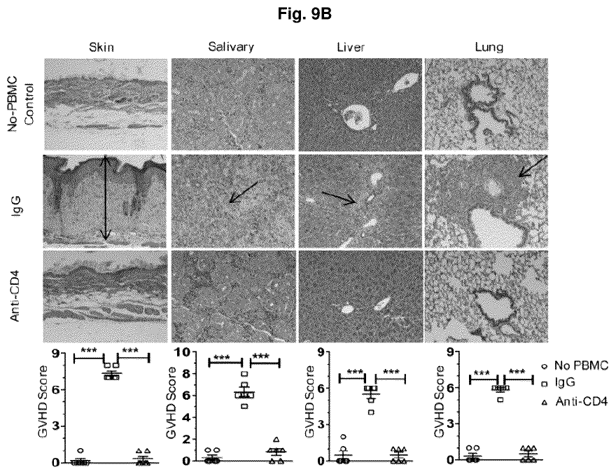

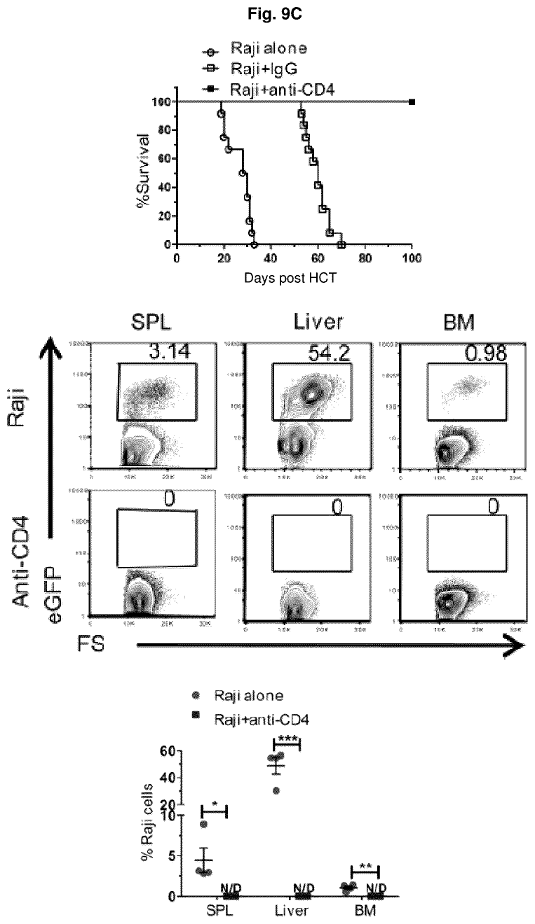

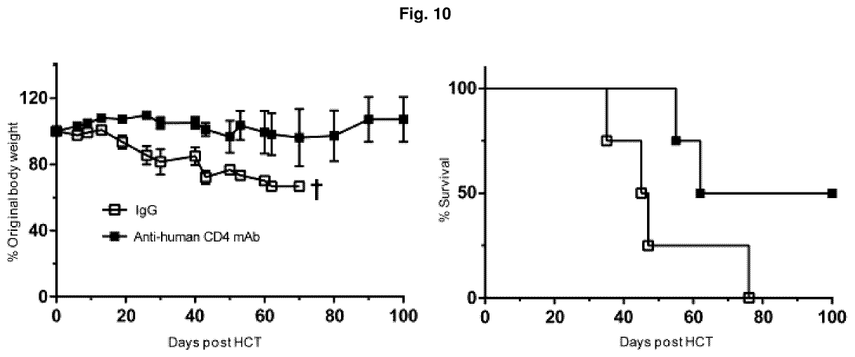

Anti-CD4 treatment effectively prevented xenogeneic GVHD in experiments with 3 of the 4 donors, and the 12 GVHD-free anti-CD4-treated NSG recipients survived for more than 100 days after PBMC injection (FIG. 9A). With cells from one donor, anti-CD4 mAb treatment was only partially effective in preventing xenogeneic GVHD (FIG. 10). IgG-treated control NSG recipients all developed GVHD with weight-loss, ruffled fur and hair-loss, and all died by ˜60 days after PBMC injection (P<0.01, FIG. 9A). Anti-CD4 treatment prevented GVHD target tissue damage in the skin, salivary gland, liver and lung (P<0.01, FIG. 9B).

In GVL experiments, control recipients given Raji cells alone all died with progressive tumor growth by 35 days. NSG mice given Raji cells and human PBMC were treated with IgG or anti-CD4 mAb. All 12 GVHD-free anti-CD4-treated mice survived for more than 100 days after PBMC injection (P<0.01, FIG. 9C), but IgG-treated mice all died with GVHD by ˜65 days after PBMC injection. Control NSG mice that died with progressive tumor growth had Raji cell infiltration in the spleen, liver and bone marrow, while the anti-CD4-treated GVHD-free NSG mice had no detectable tumor cells in these tissues (P<0.01, FIG. 9C). These results suggest that antibody-mediated in vivo depletion of donor CD4+ T cells immediately after HCT may be able to prevent GVHD while preserving GVL effects after allogeneic HCT in humans.

### Example 2: Effects of Depletion of Donor CD4+ T Cells on IFN-γ and IL-2

This example demonstrates that depletion of donor CD4+ T cells immediately after HCT increased serum IFN-γ but decreased serum IL-2 concentrations.

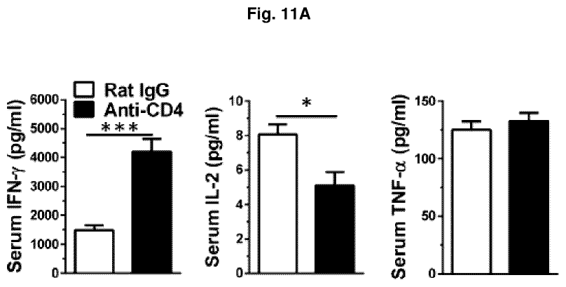

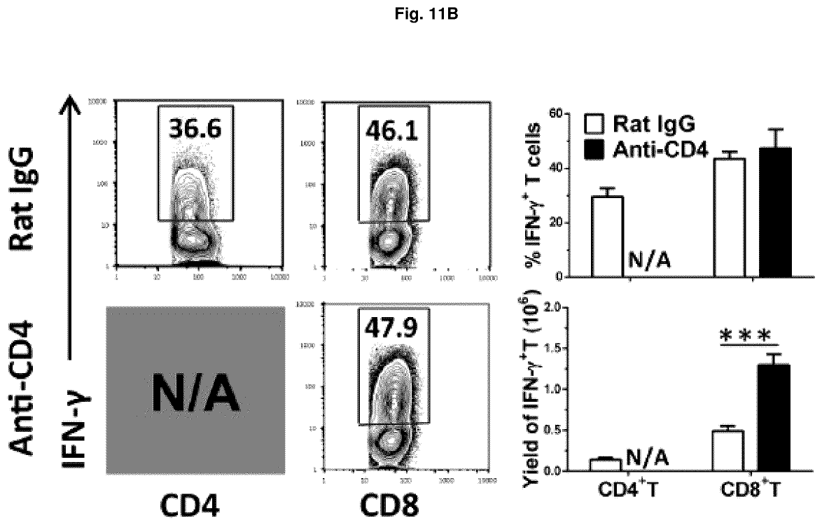

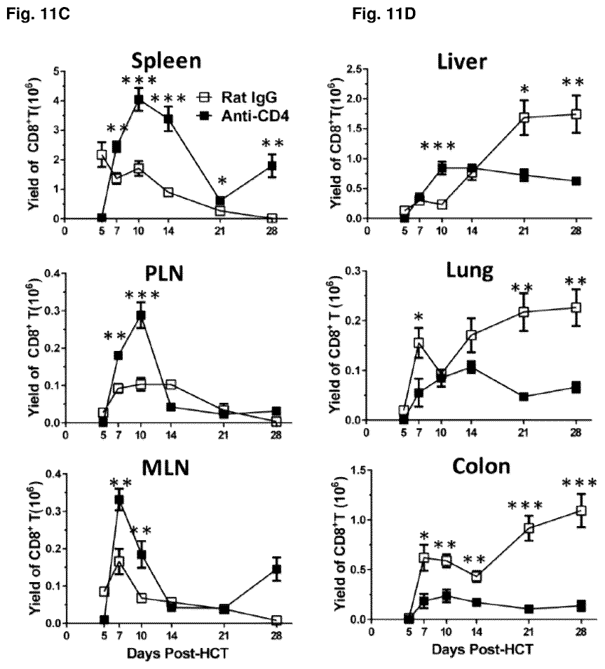

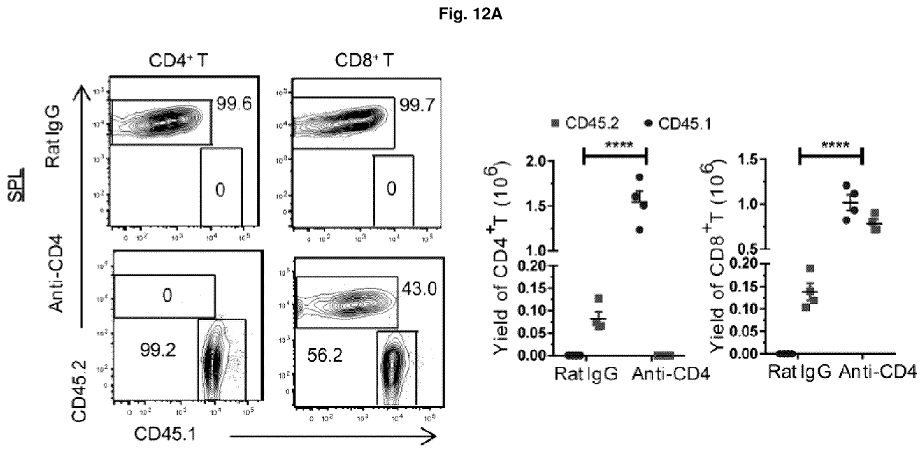

In experiments with C57BL/6 donors and BALB/c recipients, how in vivo depletion of donor CD4+ T cells immediately after HCT prevented acute GVHD while preserving GVL effects was explored. High serum levels of IFN-γ and TNF-α have been associated with acute GVHD (41). Contrary to expectation, depletion of donor CD4+ T cells increased serum IFN-γ concentrations approximately 3-fold at 7 days after HCT (p<0.001). Serum IL-2 concentrations decreased by ˜50% (p<0.05), and serum TNF-α concentrations showed no significant differences from baseline (FIG. 11A). The increased serum levels of IFN-γ is attributable to expansion of donor CD8+ T cells in lymphoid tissues, because the number of IFN-γ+CD8+ T cells in the spleen of anti-CD4-treated recipients was ˜3 fold higher than in rat IgG-treated recipients (p<0.001), although the percentage of IFN-γ+ cells among CD8+ T cells was similar in the two groups (FIG. 11B). These results suggest that in vivo depletion of CD4+ T cells immediately after HCT may expand IFN-γ-producing CD8+ T cells in lymphoid tissues.

### Example 3: Effects of Depletion of Donor CD4+ T Cells on the Numbers of Donor CD8+ T Cells in Lymphoid Tissues

This example demonstrates that depletion of donor CD4+ T cells immediately after HCT increased the numbers of donor CD8+ T cells in lymphoid tissues.

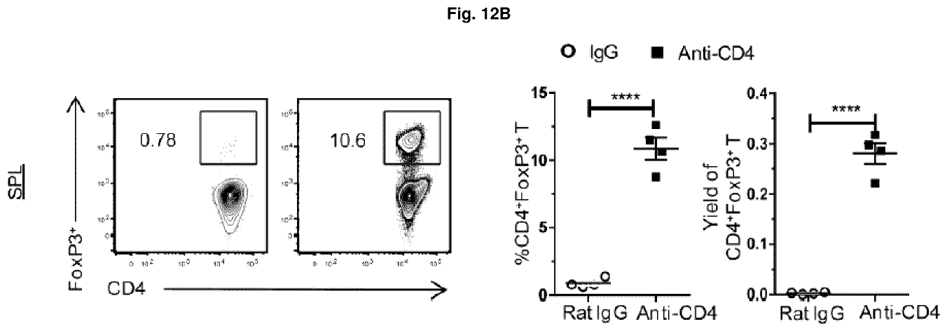

Next, the effects of in vivo CD4+ T cell depletion on donor CD8+ T cell expansion and tissue distribution were kinetically evaluated. At 5 days after HCT, the numbers of H-2Kb+ donor-type CD8+ T cells in the spleen and MLN were lower in anti-CD4-treated recipients than in rat IgG-treated recipients (P<0.01, FIG. 11C). From 7-10 days after HCT, the numbers of donor CD8+ T cells in the spleen, PLN, and MLN of anti-CD4-treated recipients were ˜3 fold higher than in the control IgG-treated recipients (p<0.01), although the numbers subsided and differences diminished between the two groups by 14-21 days after HCT (FIG. 11C). By day 28, donor CD8+ T cells expanded again in lymphoid tissues of anti-CD4-treated recipients, but not in IgG-treated recipients, and IgG-treated recipients showed severe lymphopenia (FIG. 11C). Furthermore, by using congenic markers (CD45.2 for the injected T cells and CD45.1 for T cells that were generated de novo from the donor marrow), it was found that at 28 days after HCT, CD4+ and CD8+ T cells in the spleen of IgG-treated recipients were almost all derived from CD45.2+ mature T cells in the graft. In contrast, CD4+ T cells in the spleen of anti-CD4-treated recipients were almost all derived from the CD45.1+ donor marrow, while CD8+ T cells originated from both the injected CD45.2+ T cells and the CD45.1+ donor marrow (FIG. 12A). The yield of total CD4+ and CD8+ T cells in the spleen of IgG-treated recipients was significantly lower than in anti-CD4-trated recipients (P<0.01, FIG. 12A). Very few Foxp3+ Treg cells derived from the injected CD4+ T cells were present in IgG-treated recipients, but Treg cells represented ˜10% of CD4+ T cell population derived from the donor marrow in anti-CD4-treated recipients (FIG. 12B). These results indicate that IgG-treated recipients developed acute GVHD and lymphopenia. A single injection of anti-CD4 effectively depletes the injected CD4+ T cells and augments de novo regeneration of both CD4+ and CD8+ T cells, as well as Treg cells.

### Example 4: Effects of Depletion of Donor CD4+ T Cells on the Numbers of Donor CD8+ T Cells in Different Organs

This example demonstrates that depletion of donor CD4+ T cells immediately after HCT decreased the numbers of donor CD8+ T cells in the intestine and lung but not in the liver.

At 5 days after HCT, only a few donor CD8+ T cells infiltrated the colon, lung and liver, with no difference between recipients treated with IgG or anti-CD4. From day 7 to day 28 after HCT, the numbers of donor CD8+ T cells in the colon were markedly lower in anti-CD4-treated recipients than in IgG-treated recipients (p<0.01, FIG. 11D). The pattern in the lung was similar to that in the colon. In the liver, the numbers of donor CD8+ T cells were higher in anti-CD4-treated recipients than in IgG-treated recipients at 10 days after HCT (p<0.01), but by day 21, the numbers of CD8+ T cells in IgG-treated recipients surpassed the numbers in anti-CD4-treated recipients (p<0.01, FIG. 11D). The expansion of donor CD4+ and CD8+ T cells in GVHD target tissues of IgG-treated recipients was associated with recurrence of GVHD (FIGS. 11D and 4A).

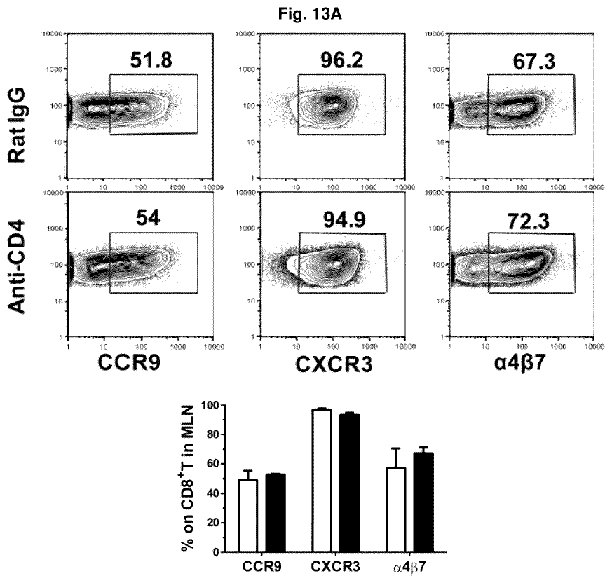

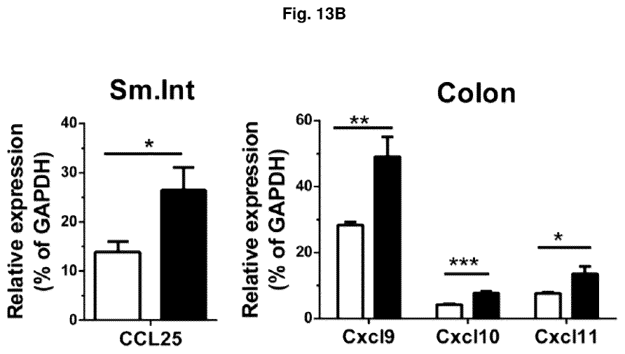

It was previously reported that donor T cell infiltration of gut tissues is regulated by their expression of gut tissue-specific homing and chemokine receptors (α4β7, CCR9, CXCR3), and by tissue release of the corresponding chemokines (CCL25 and Cxcl9-11) (42-45). By 7 days after HCT, more than 92% of the donor-type CD8+ T cells expressed a CD44hiCD62lo effector phenotype in both rat-IgG-treated and anti-CD4-treated recipients, indicating that CD8+ T cells were fully activated in both groups. Although donor CD8+ T cell infiltration of intestinal tissues (i.e., colon) was markedly decreased in anti-CD4-treated recipients at 7 days after HCT (FIG. 11C), donor CD8+ T cells did not show any significant reduction in the expression of α4β7, CCR9 or CXCR3 (FIG. 13A). Expression of CCL25 in the small intestine and expression levels of Cxcl9-11 in the colon were higher in anti-CD4-treated recipients than in IgG-treated recipients (p<0.05, FIG. 13B). These results suggest that reduction of gut tissue infiltration by donor CD8+ T cells after depletion of donor CD4+ T cells is unlikely due to decreased CD8+ T cell migration into gut tissues.

### Example 5: Effects of Depletion of Donor CD4+ T Cells on Donor CD8+ T Cell Apoptosis

This example demonstrates that depletion of donor CD4+ T cells immediately after HCT augmented donor CD8+ T cell apoptosis in the intestine and anergy/exhaustion in the liver, but not in the spleen.

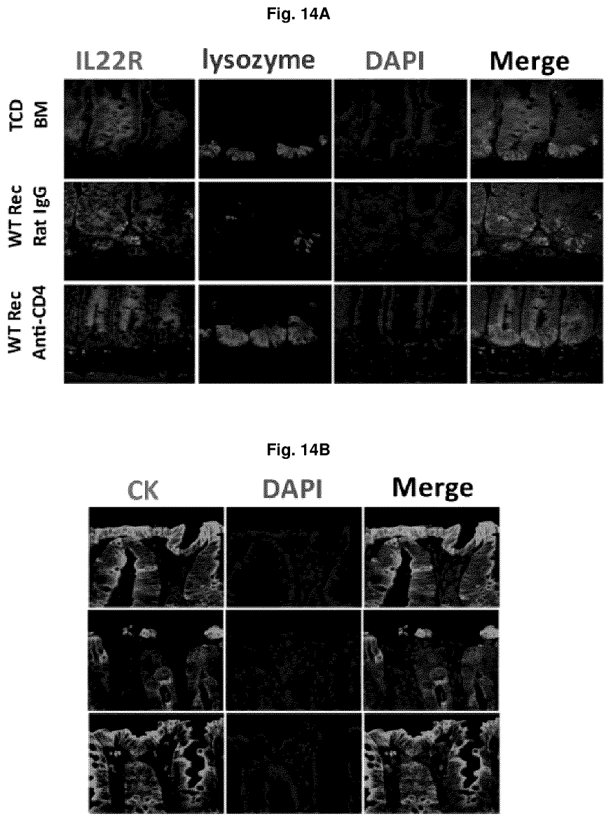

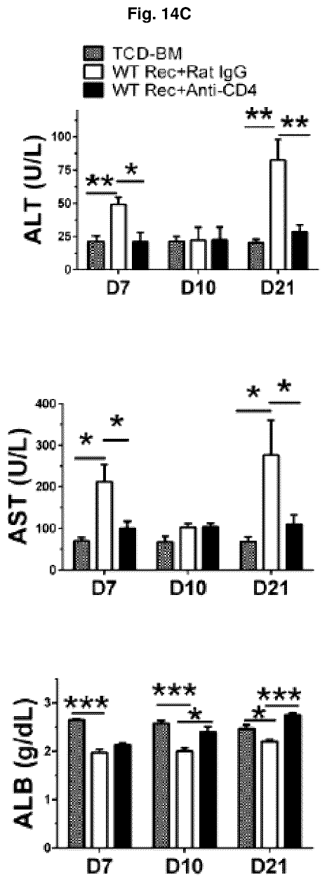

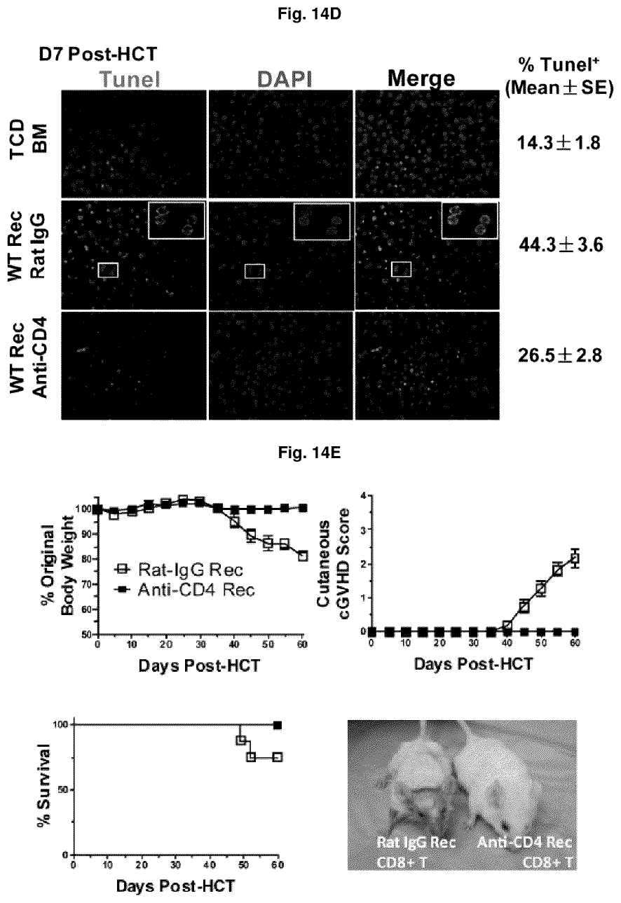

The mechanisms was explored, whereby anti-CD4-treated GVHD-free recipients had reduced numbers of donor CD8+ T cell numbers in the colon and similar or higher numbers in the liver, while having increased numbers of donor CD8+ T cells in the spleen, as shown in FIG. 11. In the pathogenesis of GVHD, alloreactive donor T cells damage Paneth cells in the small intestine and disrupt epithelial junctions in the colon (46, 47). Consistently, anti-CD4-treated recipients without signs of diarrhea showed little damage to Paneth cells in the small intestine and little disruption of epithelial junctions in the colon (FIGS. 14A & 14B).

Alloreactive T cell infiltration also plays a critical role in damage to the liver (3). Although the numbers of liver infiltrating CD8+ cells were markedly higher in anti-CD4-treated recipients than in control IgG-treated recipients on day 10 after HCT (FIG. 11D), anti-CD4-treated recipients appeared to have little damage to liver or evidence of hepatocyte apoptosis, in contrast to IgG-treated control recipients (P<0.01, FIGS. 14C and 14D). Furthermore, liver infiltrating CD8+ T cells from IgG-treated recipients at day 21 after HCT induced GVHD in secondary adoptive recipients, while CD8+ T cells from anti-CD4-treated recipients did not (FIG. 14E). These results suggest that liver infiltrating CD8+ T cells may be anergic or exhausted, such that they become non-pathogenic.

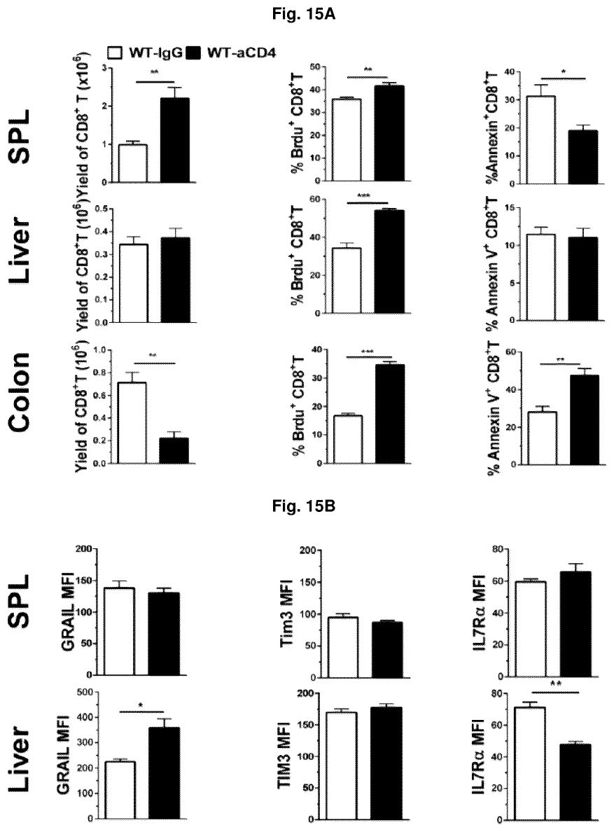

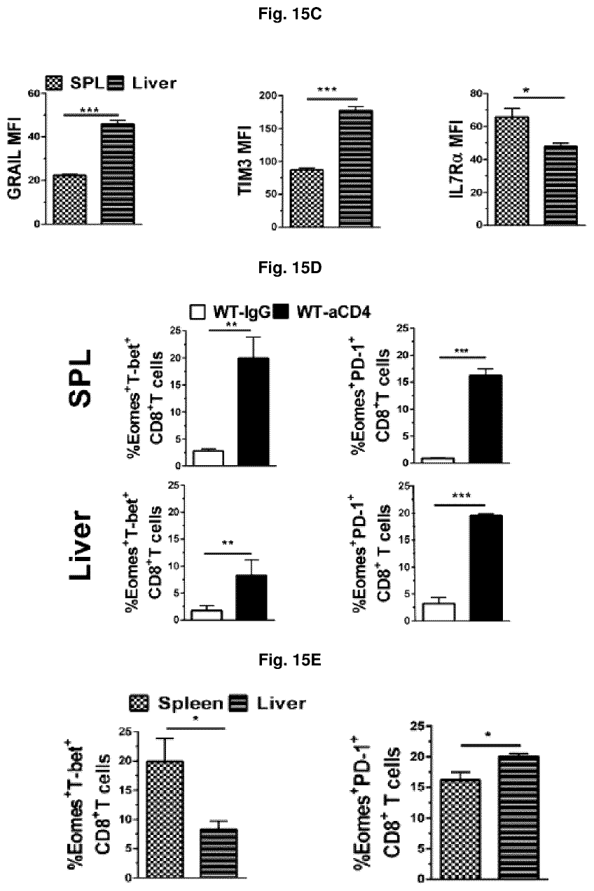

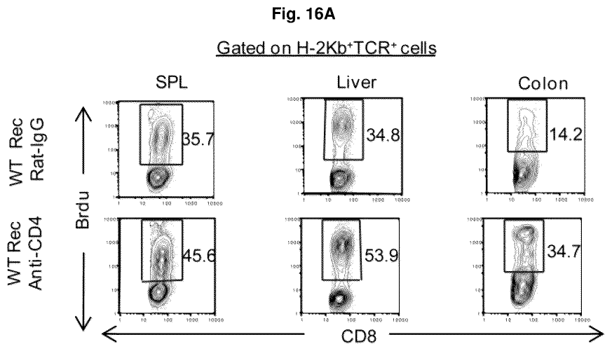

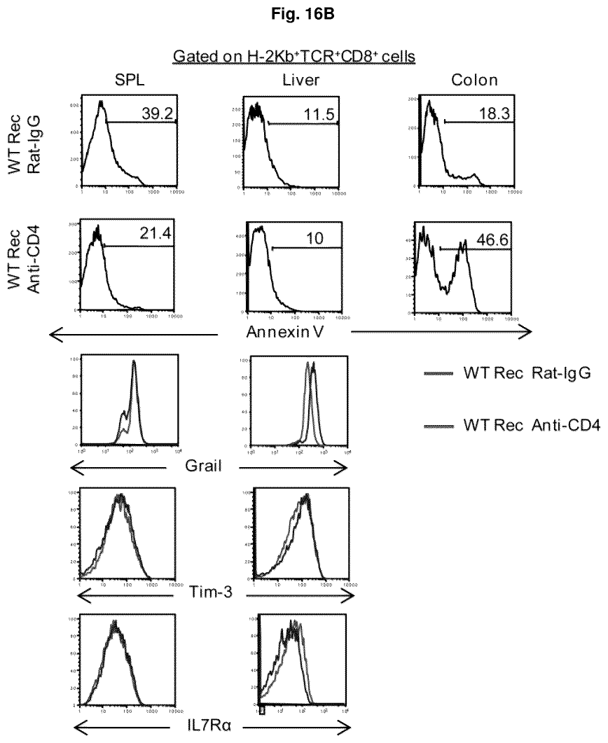

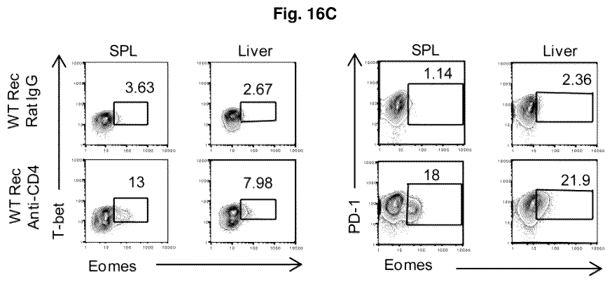

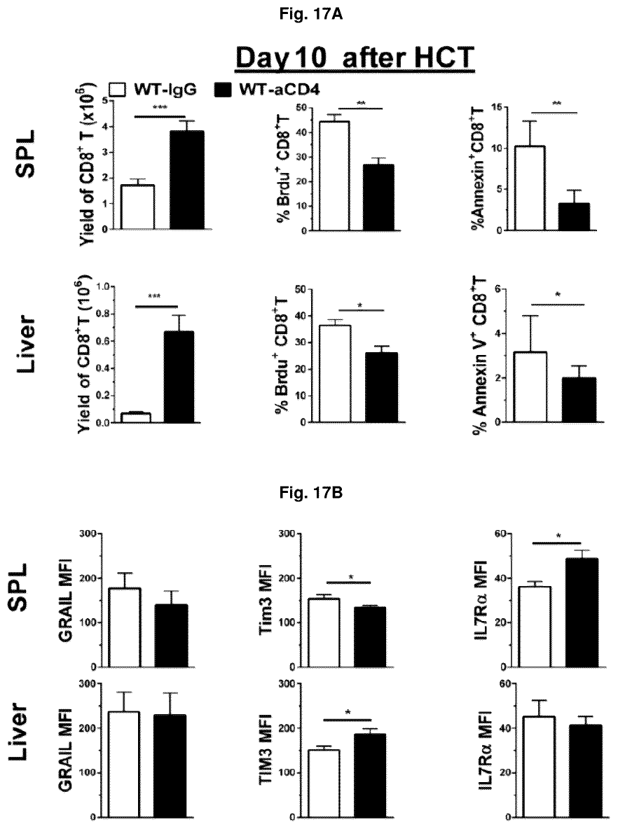

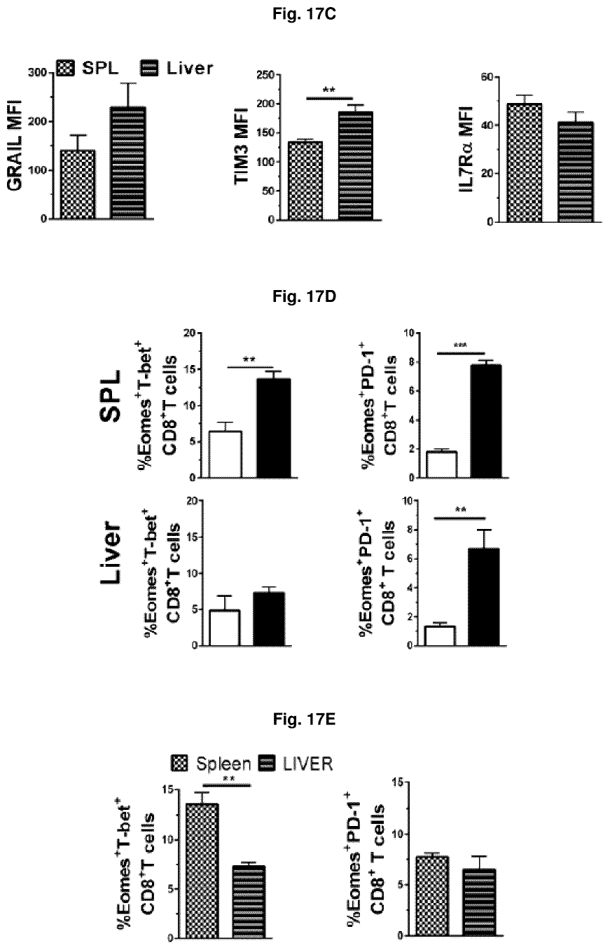

Therefore, the proliferation and apoptosis of donor CD8+ T cells in the spleen, liver and colon tissues at 7 and 10 days after HCT were compared. At day 7, in vivo BrdU labeling showed that donor CD8+ T cells had significantly faster proliferation in the spleen, liver, and intestine tissues in anti-CD4-treated recipients as compared to IgG-treated recipients (P<0.01, FIG. 15A, middle column, and FIG. 16A). In contrast, apoptosis of donor CD8+ T cells was markedly reduced in the spleen (P<0.01), not significant changed in the liver, and markedly increased in the colon (P<0.01) in anti-CD4-treated recipients as compared to IgG-treated recipients (FIG. 15A, right column, and FIG. 16B). By day 10, donor CD8+ T cells in the spleen and liver of anti-CD4-treated recipients no longer proliferated better, although apoptosis rate was still lower (FIG. 17A). Therefore, the increased proliferation and reduced apoptosis led to the increased numbers of donor CD8+ T cells in the spleen and liver of anti-CD4-treated recipients immediately after HCT.

To evaluate anergy and exhaustion of donor CD8+ T cells, the CD8+ T cell expression levels (mean fluorescent index, MFI) of the anergy/exhaustion-related markers including Grail, Tim-3 and IL-R7α were compared. As compared to IgG-treated recipients, the CD8+ T cells from the spleen of anti-CD4-treated recipients did not have significant change in their expression of Grail, Tim-3 or IL-7Rα on day 7 (FIGS. 15B & 16C), but they had significantly down-regulated expression of Tim-3 and upregulated expression of IL-7Rα on day 10 (FIG. 17B). In contrast, the CD8+ T cells from the liver of anti-CD4-treated recipients had significantly increased expression of Grail and down-regulated expression of IL-7Rα on day 7, although the changes appeared to be small (FIGS. 15B & 16C), and on day 10 after HCT, they had upregulated expression of Tim-3 (FIG. 17B). In addition, when comparing CD8+ T cells from the liver and spleen of anti-CD4-treated recipients, CD8+ T cells from the liver expressed significantly higher levels of Grail and Tim-3 and lower levels of IL-7Rα at 7 days after HCT (P<0.05, FIG. 15C); and higher levels of Tim-3 persisted at day 10 (P<0.01, FIG. 17C). These results suggest that donor CD8+ T cells in the liver of anti-CD4-treated recipients become anergic and exhausted by 7 to 10 days after HCT, while those in the spleen do not.

Eomes regulates CD8+ T differentiation (48). Eomes+T-bet+ CD8+ T cells are effector cells with strong cytolytic function, while Eomes+PD-1+ CD8+ T cells are terminally differentiated exhausted cells (49, 50). Therefore, the impact of depletion of CD4+ T cells on CD8+ T expression of Eomes, T-bet, and PD-1 in the spleen and liver at 7 and 10 days after HCT were evaluated. CD8+ T cells from the spleen and liver of anti-CD4-treated recipients had significant increase in percentages of Eomes+T-bet+ and Eomes+PD-1+ cells, as compared to control IgG-treated recipients at days 7 and 10 after HCT (P<0.01, FIGS. 15D, 16D and 17D). The increase of Eomes+T-bet+ cells was dominant among splenic CD8+ T cells on days 7 and 10, while the increase of Eomes+PD-1+ cells was dominant among CD8+ T cells in the liver at day 7, with no difference on day 10 (FIGS. 15E and 17E). These results indicate that anti-CD4 depletion of donor CD4+ T cells immediately after HCT leads to preferential cytotoxic differentiation of CD8+ T cells in the spleen and preferential terminal differentiation and exhaustion of CD8+ T cells in the liver.

### Example 6: Effects of Depletion of Donor CD4+ T Cells on Host-Tissue Expression of PD-L1

This example demonstrates that depletion of donor CD4+ T cells immediately after HCT allowed host-tissue expression of PD-L1 to tolerize infiltrating donor CD8+ T cells in GVHD target tissues but not in lymphoid tissues.

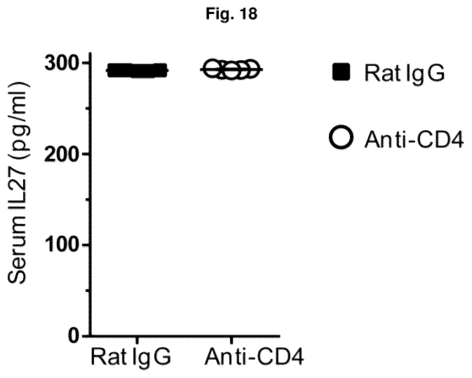

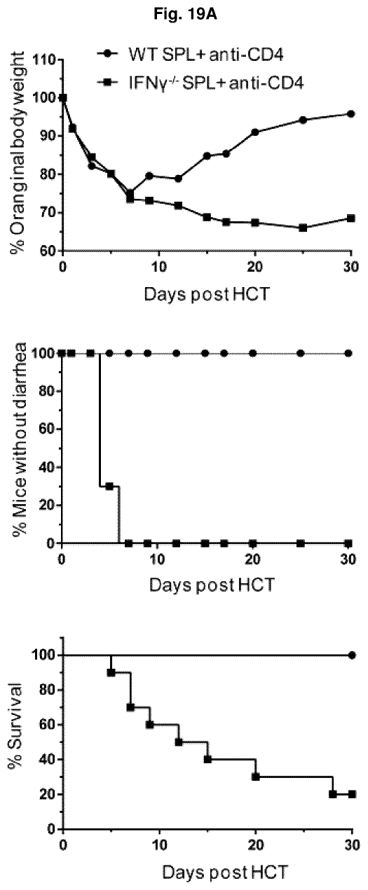

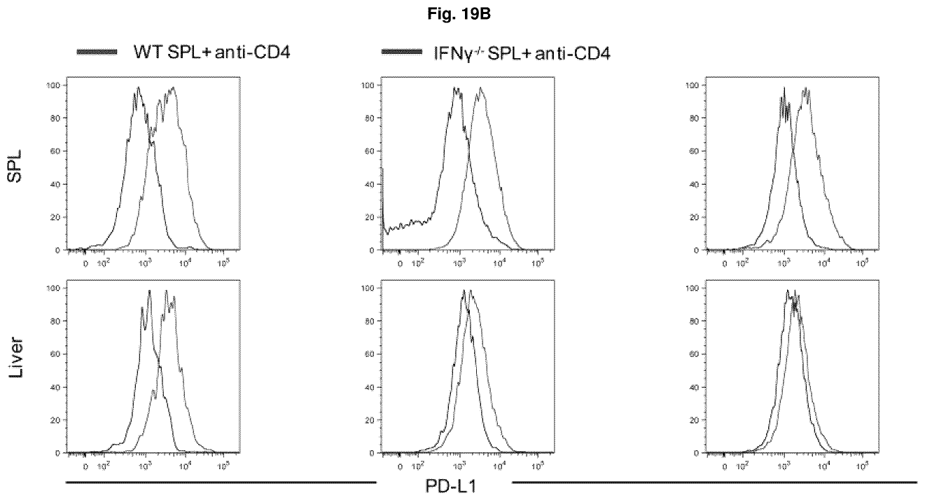

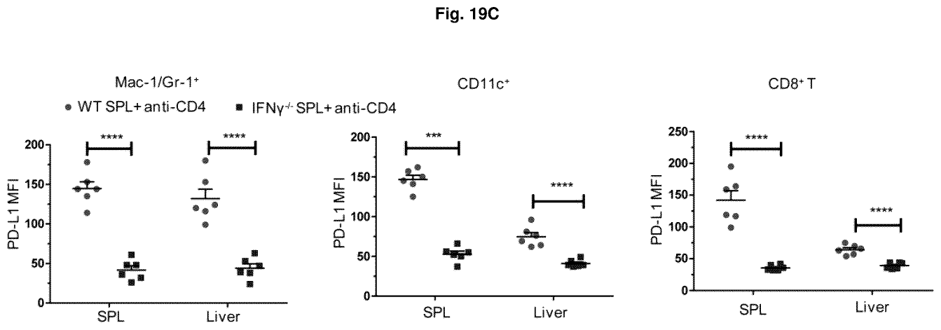

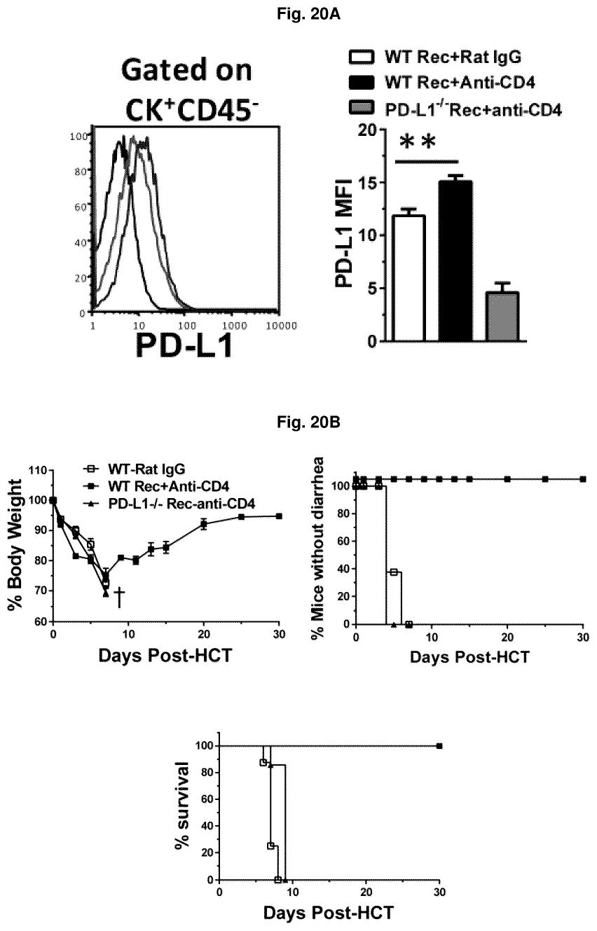

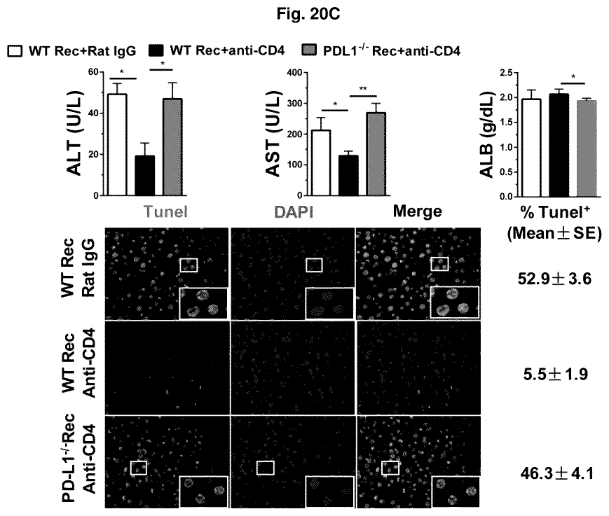

PD-L1/PD-1 interaction leads to T cell anergy and exhaustion (24), and simultaneous PD-L1/PD-1 and PD-L1/CD80 interactions augment apoptosis of activated alloreactive CD4+ T cells immediately after HCT (31). Depletion of donor CD4+ T cells increased serum levels of IFN-γ (FIG. 11A), and IFN-γ induces tissue expression of PD-L1 in GVHD target tissues (27, 29). Although IL-27 upregulates PD-L1 expression (51), no difference in serum IL-27 concentrations in recipients with or without anti-CD4-treatment was observed (FIG. 18). Thus, whether donor cell IFN-γ production and tissue expression of PD-L1 contributed to prevention of GVHD in anti-CD4-treated recipients was tested first. Spleen cells (5×106) from IFN-γ−/− and wild-type C57BL/6 donors were transplanted into lethally irradiated BALB/c recipients, as described above. Indeed, anti-CD4 treatment did not prevent acute GVHD mediated by transplants from IFN-γ−/− donors. All of recipients showed severe diarrhea and weight-loss, and ˜80% (8/10) of the recipients died by 30 days after HCT (FIG. 19). The CD8+ T cells, CD11c+ dendritic cells and Mac-1/Gr-1+ myeloid cells in the spleen and liver of recipients given IFN-γ−/− transplants all had significantly down-regulated expression of PD-L1 immediately after HCT (FIG. 19). These results suggest that IFN-γ production and tissue PD-L1 expression contribute to GVHD prevention by administration of anti-CD4 immediately after HCT.

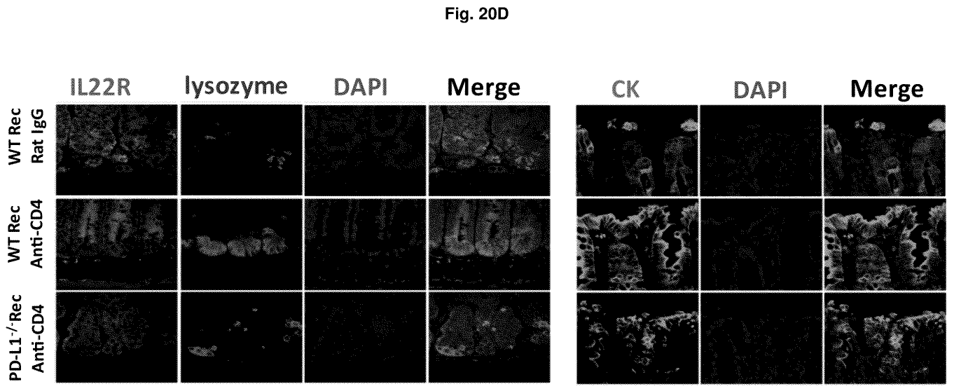

Furthermore, it was found that elevation of IFN-γ in anti-CD4-treated BALB/c recipients given wild-type C57BL/6 transplants was associated with upregulation of host intestinal epithelial cell expression of PD-L1 (FIG. 20A), and in contrast to acute GVHD-free anti-CD4-treated wild-type recipients, anti-CD4-treated PD-L1−/− recipients showed severe acute GVHD, as judged by bodyweight loss, severe diarrhea, and death within 10 days after HCT (FIG. 20B). The acute GVHD was associated with liver dysfunction, hepatocyte apoptosis, and loss of Paneth cells and colon epithelial integrity (P<0.01, FIGS. 20C and 20D). The severity of acute GVHD in PD-L1−/− recipients appeared to be similar to that of IgG-treated control WT recipients (FIGS. 20B-20D).

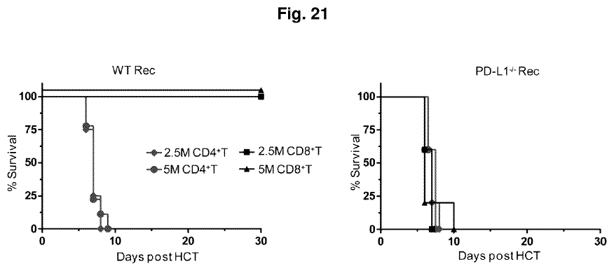

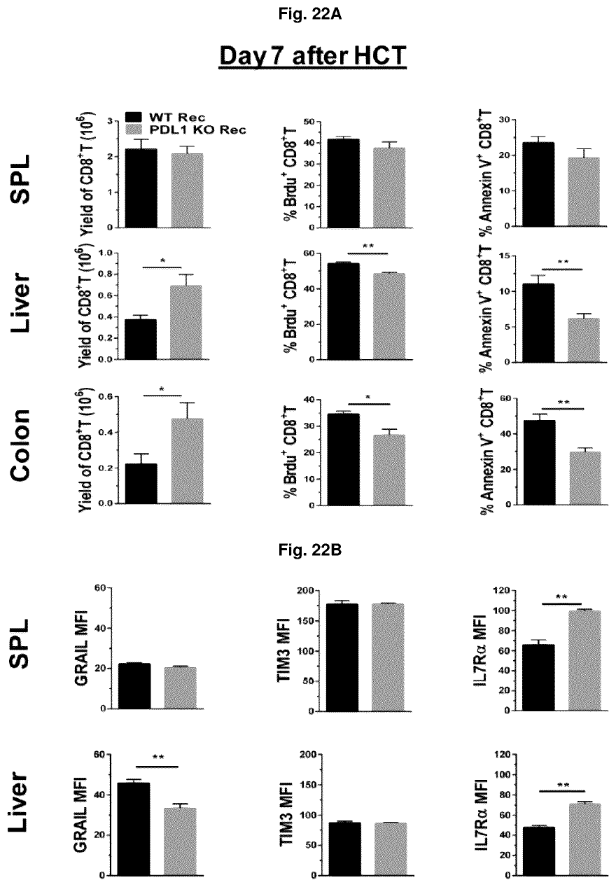

In addition, the role of host-tissue PD-L1 on acute GVHD severity induced by sorted CD4+ or CD8+ T cells was directly tested. While 2.5 or 5×106 sorted CD8+ T cells induced very little evidence of acute GVHD, the same numbers of donor CD4+ T cells induced severe lethal acute GVHD, and all the recipients died within 10 days (P<0.01, FIG. 21A). Conversely, 2.5×106 or 5×106 sorted CD8+ T cells induced severe lethal acute GVHD in PD-L1−/− recipients, and the severity was similar to that induced by the same number of donor CD4+ T cells (FIG. 21B). Taken together, these results indicate that host-tissue expression of PD-L1 plays a critical role in preventing acute GVHD mediated by donor CD8+ T cells in the absence of donor CD4+ T cells.

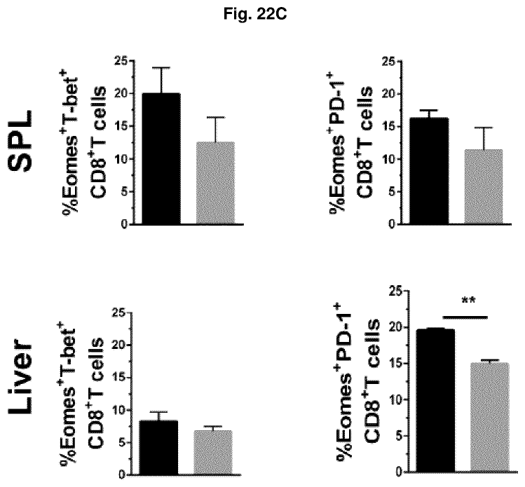

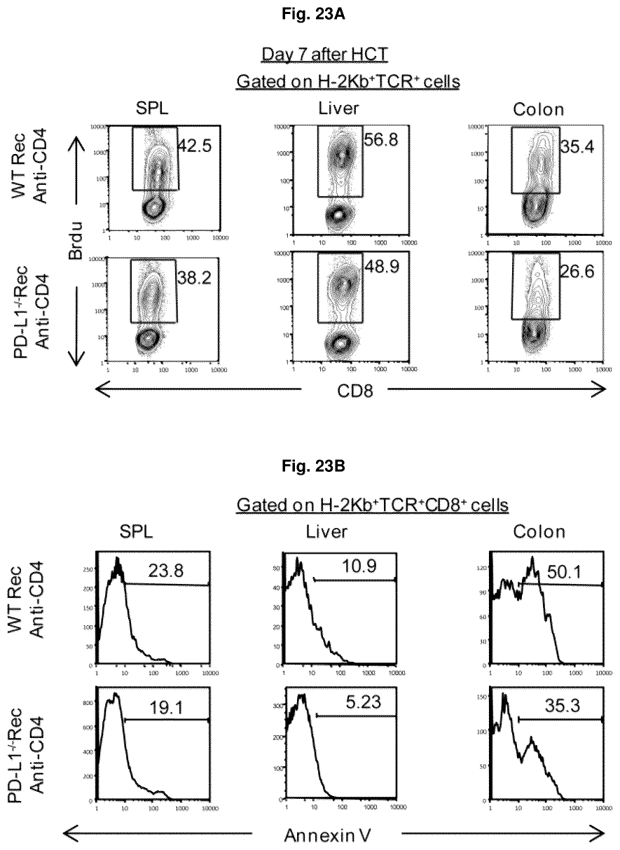

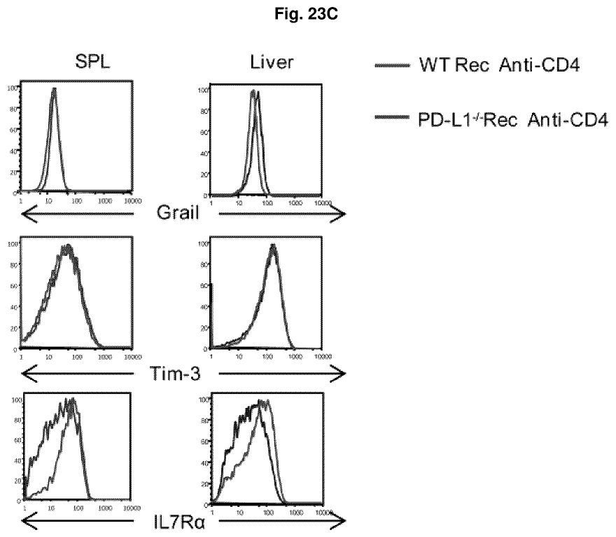

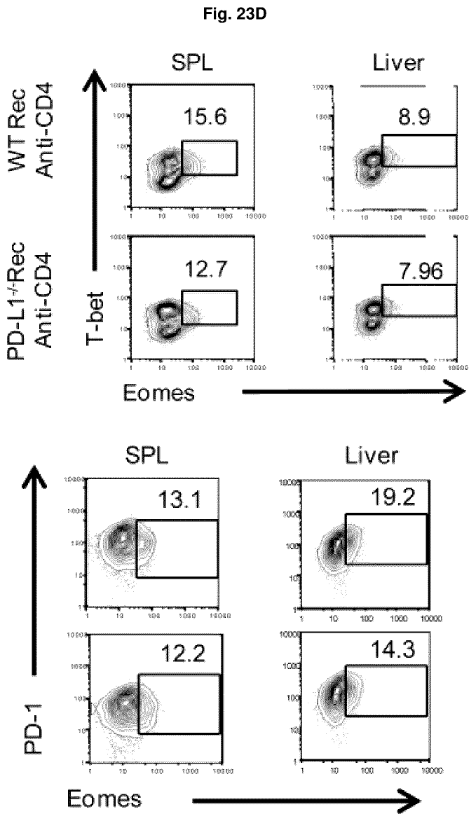

In further experiments, the effect of host tissue expression of PD-L1 on the proliferation, apoptosis and anergy/exhaustion of CD8+ T cells in the spleen, liver and colon tissues of anti-CD4-treated recipients on day 7 after HCT was evaluated. As compared to anti-CD4-treated WT recipients, anti-CD4-treated PD-L1−/− recipients had no changes in proliferation or apoptosis of donor CD8+ in the spleen (FIGS. 22A, 23A, and 23B), with no difference in the numbers of CD8+ T cells between PD-L1−/− recipients and controls. Anti-CD4-treated PD-L1−/− recipients had a significant decrease in CD8+ T proliferation and apoptosis in the liver and colon, and the reduction of apoptosis outweighed the reduction of proliferation (P<0.01, FIGS. 22A, 23A, and 23B), such that higher numbers of donor CD8+ T cells infiltrated the liver and colon of PD-L1−/− recipients as compared to controls.

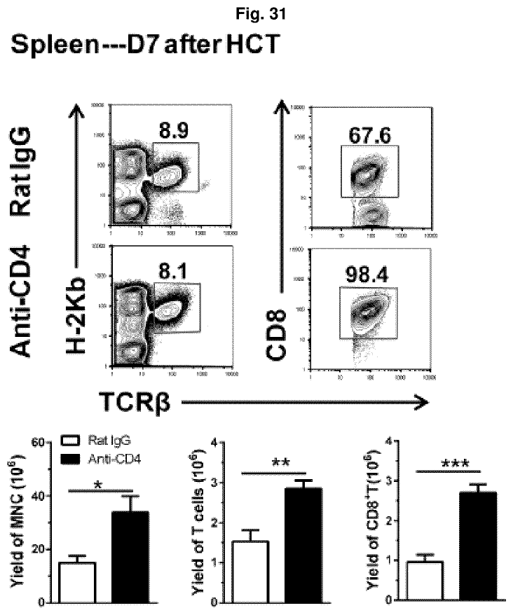

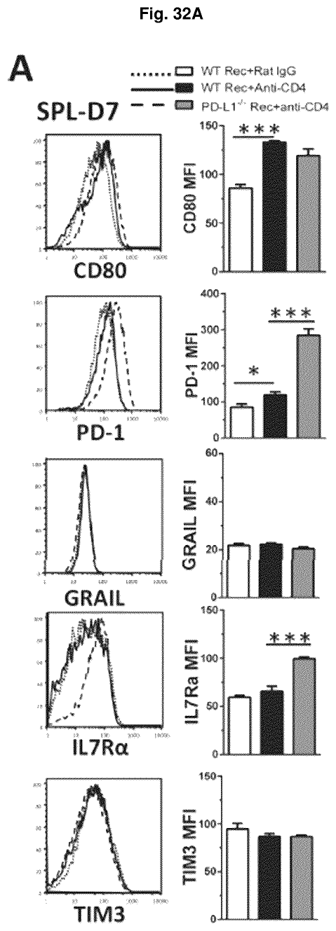

The impact of host-tissue expression of PD-L1 on donor CD8+ T expansion in the spleen on day 7 after HCT was evaluated. The numbers of splenic mononuclear cells (MNC), T cells, and CD8+ T cells were higher in anti-CD4-treated WT recipients than in rat-IgG-treated WT recipients (p<0.05-0.001, FIG. 31).

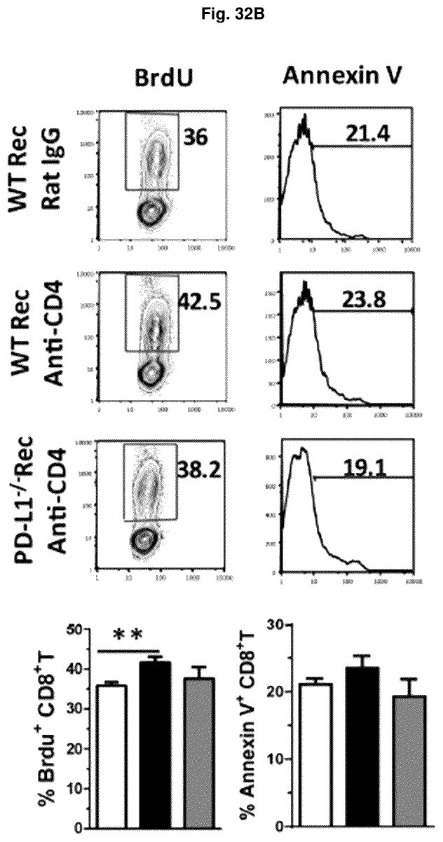

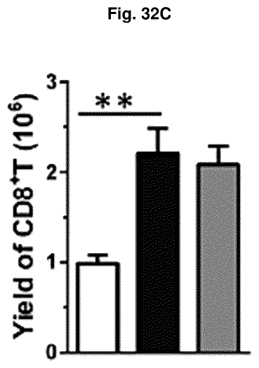

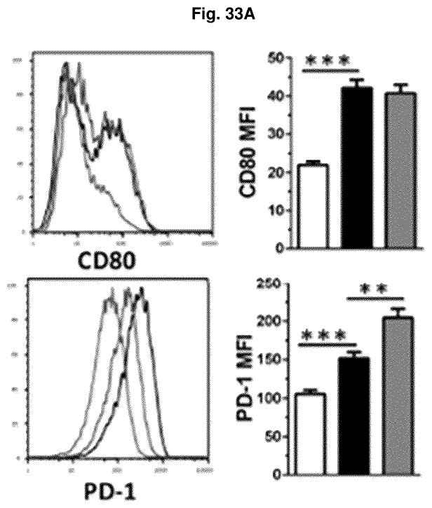

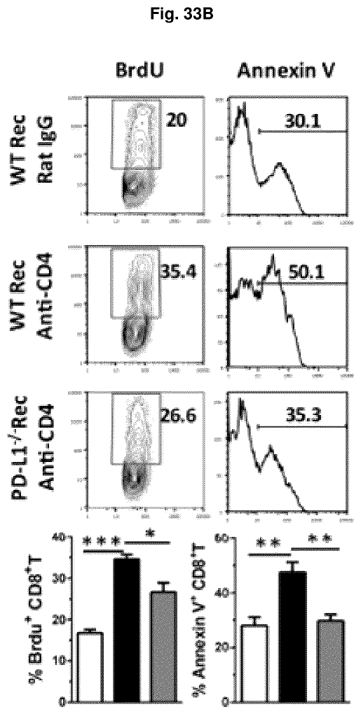

CD80 and PD-1 expression by CD8+ T cells in the spleen was higher in anti-CD4-treated WT recipients than in rat IgG-treated recipients (p<0.05-0.001, FIG. 32A), while IL-7Rα and GRAIL, and TIM3 expression was similar in the 2 groups (p>0.1, FIG. 32A). The higher expression of CD80 and PD-1 after anti-CD4 treatment was associated with increased CD8+ T cell proliferation (p<0.01) but no significant increase of apoptosis (FIG. 32B), which accounts for the higher numbers of CD8+ T cells in the spleen of anti-CD4-treated recipients compared to rat IgG-treated recipients (p<0.01, FIG. 32C).

On day 7 after anti-CD4 treatment, expression of PD-1 and IL7Rα by CD8+ T cells in the spleen was higher in PD-L1−/− recipients than in WT recipients (p<0.001, FIG. 32A). Expression of CD80, GRAIL and TIM3 after anti-CD4 treatment was not affected by the absence of PD-L1 in the recipient, and proliferation, apoptosis and the numbers of CD8+ T cells in the spleen did not differ significantly in the 2 groups (FIGS. 32B & 32C).

These results indicate that host-tissue expression of PD-L1 augments the apoptosis of infiltrating CD8+ T cells in the liver and intestine but not in the spleen of anti-CD4-treated recipients.

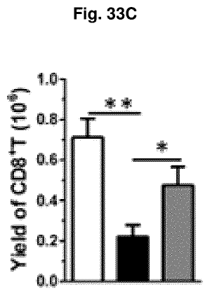

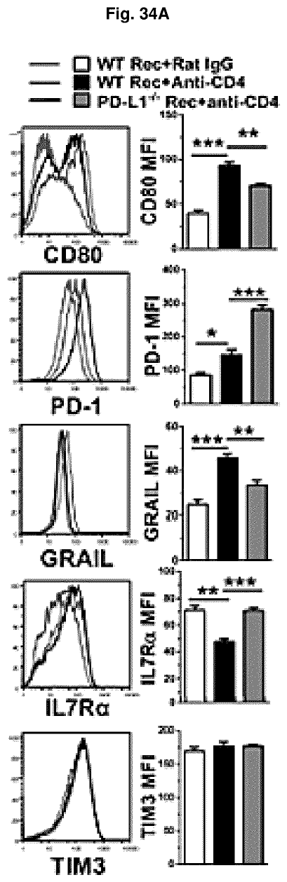

Expression of CD80 and PD-1 by infiltrating CD8+ T cells was higher in anti-CD4-treated WT and PD-L1−/− recipients than in rat IgG-treated WT recipients (p<0.001, FIG. 33A). Donor CD8+ T cell proliferation and apoptosis of infiltrating CD8+ T cells was higher in anti-CD4-treated WT than in rat IgG-treated WT recipients (p<0.01, FIG. 33B). Increased apoptosis outweighed increased proliferation, so that the numbers of infiltrating CD8+ T cells was lower in anti-CD4-treated WT recipients than in rat IgG-treated WT recipients (p<0.01, FIG. 33C). In PD-L1−/− recipients, the proliferative effect of anti-CD4 treatment on infiltrating CD8+ T cells was attenuated, and the pro-apoptotic effect of anti-CD4 treatment on infiltrating CD8+ T cells was blocked. As a result, the numbers of infiltrating CD8+ T cells after anti-CD4 treatment were higher in PD-L1−/− recipients than in WT recipients (p<0.05, FIGS. 33B and 33C).

These results demonstrate that recipient tissue expression of PD-L1 contributed to increase of apoptosis of colon infiltrating CD8+ T cells and prevention of intestinal GVHD after CD4+ T cell depletion.

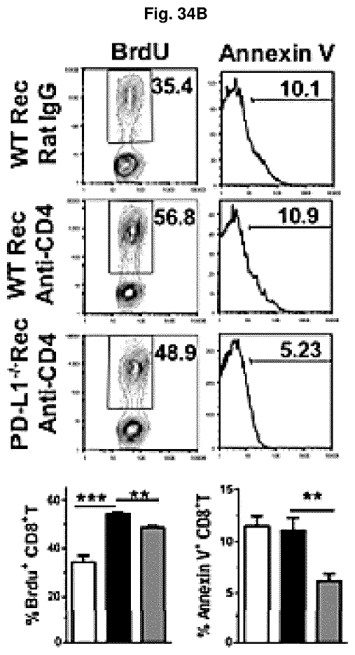

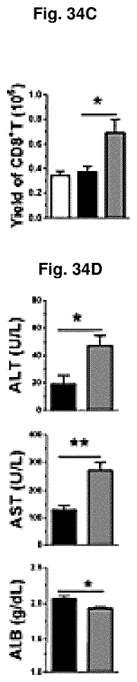

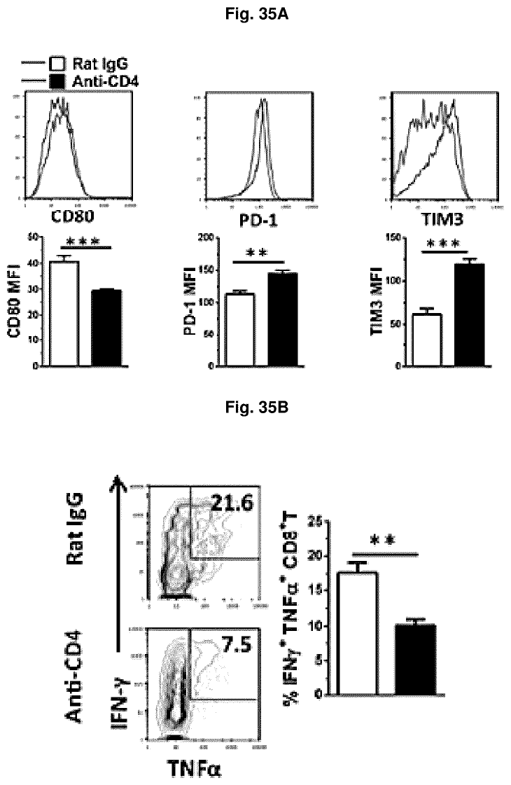

The impact of host-tissue expression of PD-L1 on anergy and exhaustion of CD8+ T cells infiltrating the liver at day 7 after HCT was evaluated. Anergic CD8+ T cells upregulate expression of GRAIL and down-regulate expression of IL-7Rα without significant changes in TIM3 expression, while exhausted CD8+ T cells express high levels of both PD-1 and TIM3 (72-75). Anergic and exhausted T cells gradually lose proliferative capacity and effector function (e.g., production of IFN-□) (72, 73). As compared to rat-IgG-treated recipients, liver infiltrating CD8+ T cells of anti-CD4-treated recipients expressed higher levels of CD80, PD-1, and GRAIL (p<0.01), lower levels of IL-7Rα (p<0.01), and similar levels of TIM3, (FIG. 34A). In the colon, increased expression of CD80 and PD-1 by infiltrating CD8+ T cells in anti-CD4 recipients was associated with increased proliferation and apoptosis (FIG. 33B). In the liver, upregulation of CD80 and PD-1 by infiltrating CD8+ T cells in anti-CD4-treated recipients was associated with increased proliferation (p<0.01) but not with increased apoptosis (FIG. 34B). Increased proliferation of infiltrating CD8+ T cells was associated with upregulated expression of GRAIL and down-regulated expression of IL-7R (FIG. 34A).

These results suggest that the infiltrating CD8+ T cells in the liver of anti-CD4-treated recipients immediately after HCT were becoming anergic.

In the absence of recipient PD-L1, expression of GRAIL by CD8+ T cells was not significantly upregulated, and expression of IL-7Rα was not down-regulated after anti-CD4 treatment (FIG. 34A), indicating that PD-L1 expression in the recipient is required for development of anergy in CD8+ T cells infiltrating the liver. In the absence of recipient PD-L1, T cells proliferated somewhat less rapidly, but apoptosis of CD8+ T cells was decreased by ˜50% after anti-CD4 treatment (p<0.01, FIG. 34B). In keeping with these results, the number of CD8+ T cells infiltrating the liver of anti-CD4-treated recipients was significantly higher in PD-L1−/− recipients than in WT recipients (p<0.05, FIG. 34C). Serum transaminase concentrations were also higher but serum ALB was lower in PD-L1−/− recipients than in WT recipients (p<0.05) (FIG. 34D).

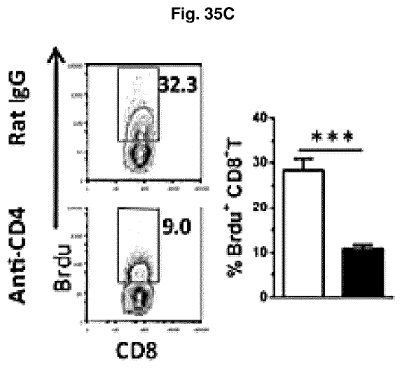

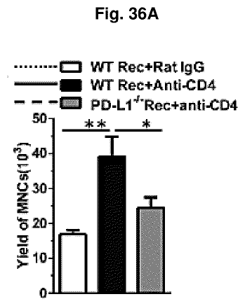

At 21 days after HCT, CD8+ T cells infiltrating the liver were exhausted in anti-CD4-treated recipients but not in rat-IgG-treated recipients, as judged by their up-regulation of PD-1 and TIM-3 (p<0.01, FIG. 35A), the marked reduction of intracellular IFN-□ and TNF-α expression (p<0.01, FIG. 35B), and the loss of proliferation (p<0.01, FIG. 35C.

Taken together, these results show that CD8+ T cells infiltrating the liver in anti-CD4-treated recipients immediately after HCT became progressively anergic and exhausted through mechanisms dependent on expression of PD-L1 in the recipient.

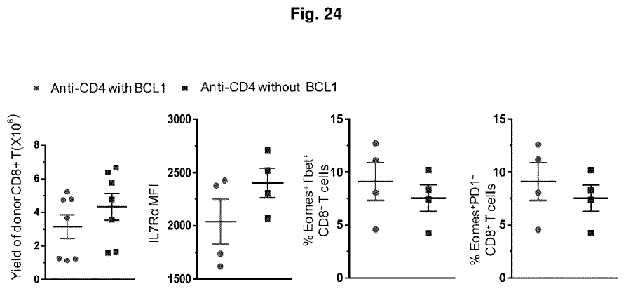

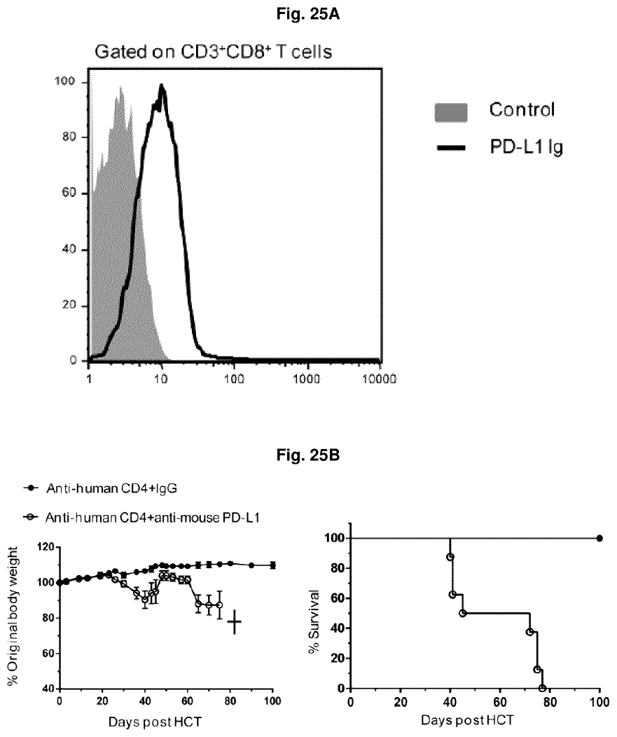

The expression levels (MFI) of Grail, Tim-3, IL-7Rα and percentage of Eomes+T-bet+CD8+ and Eomes+PD-1+CD8+ T cells in the spleen and liver of PD-L1−/− recipients and controls at 7 days after HCT were compared. The absence of host-tissue expression of PD-L1 did not significantly change donor CD8+ T expression of Grail or Tim-3 in the spleen, although expression of IL-7Rα was higher in PD-L1−/− recipients than in WT recipients (FIGS. 22B and 23C). On the other hand, the absence of host-tissue expression of PD-L1 reduced expression of Grail and increased expression of IL-7Rα by CD8+ T cells in the liver, with no significant changes in Tim-3 expression (FIGS. 22B and 23C). The absence of host tissue PD-L1 did not significantly change the percentages of Eomes+T-bet+or Eomes+PD-1+ CD8+ T cells in the spleen. The absence of host tissue PD-L1 did not significantly change the percentage of Eomes+T-bet+CD8+ T cells in the liver, but the percentage of Eomes+PD-1+ CD8+ T cells in the liver was lower in PD-L1−/− recipients compared to wild-type recipients (FIGS. 22C and 23D). In addition, the presence of BCL1 tumor cells did not have a significant impact on the induction of tolerance in donor CD8+ T cells infiltrating the liver (FIG. 24), suggesting that donor CD8+ T cells in the liver tissues are able to eliminate the infiltrating tumor cells before becoming completely anergic or exhausted. Taken together, these results show that in anti-CD4-treated allogeneic mouse recipients, host-tissue expression of PD-L1 plays an important role in the induction of anergy, exhaustion and apoptosis of donor CD8+ T cells infiltrating the liver but not in the spleen.

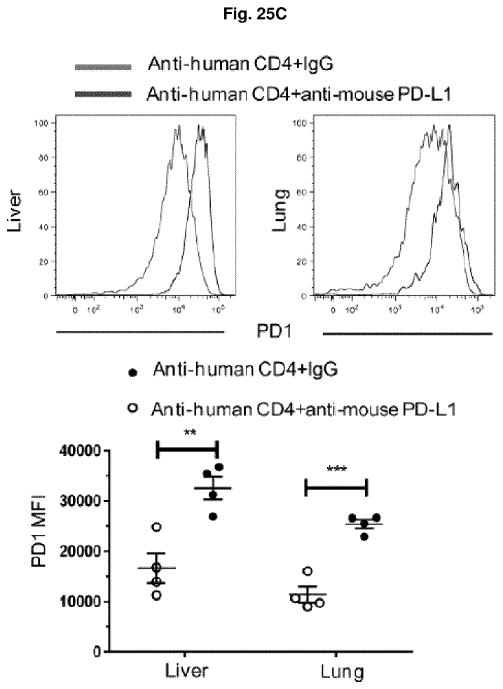

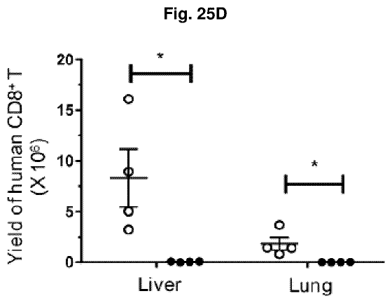

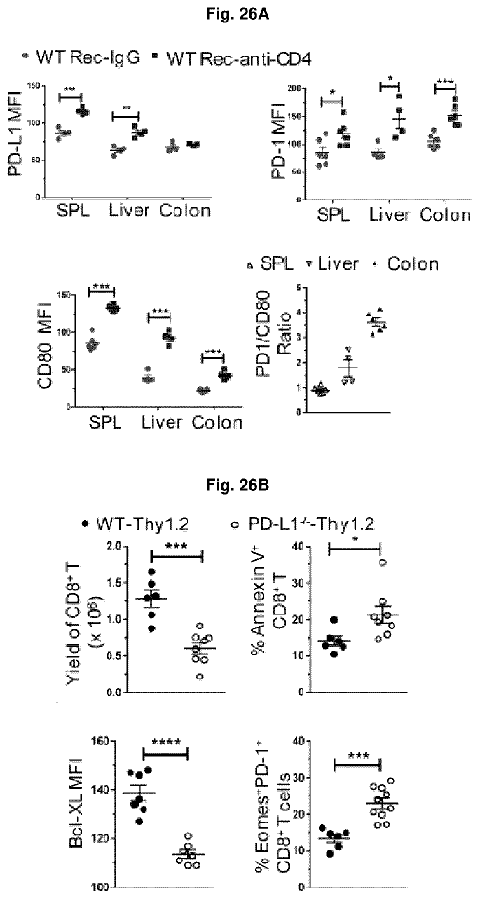

Furthermore, it was found that human T cells could interact with mouse PD-L1 (FIG. 25A), and blockade of PD-L1 interaction with its receptors by administration of anti-mouse-PD-L1 led to development of lethal xenogeneic GVHD in anti-human-CD4-treated NSG-recipients given human PBMC, whereas the control recipients without anti-PD-L1 blockade showed no signs of xenogeneic GVHD (FIG. 25B). Blockade with anti-PD-L1 led to significant down-regulation of PD-1 expression by CD8+ T cells and significant augmentation of CD8+ T cell expansion in the liver and lung (FIGS. 25C & 25D). These results suggest that tissue expression of PD-L1 contributes to tolerization of human donor CD8+ T cells in xenogeneic recipients in the absence of human CD4+ T cells.

### Example 7: Effects of Depletion of Donor CD4+ T Cells on Expression of PD-L1 and CD80

This example demonstrates that depletion of donor CD4+ T cells immediately after HCT led to donor CD8+ T cell upregulated expression of PD-L1 and CD80 in lymphoid tissues, which preserved GVL effects.

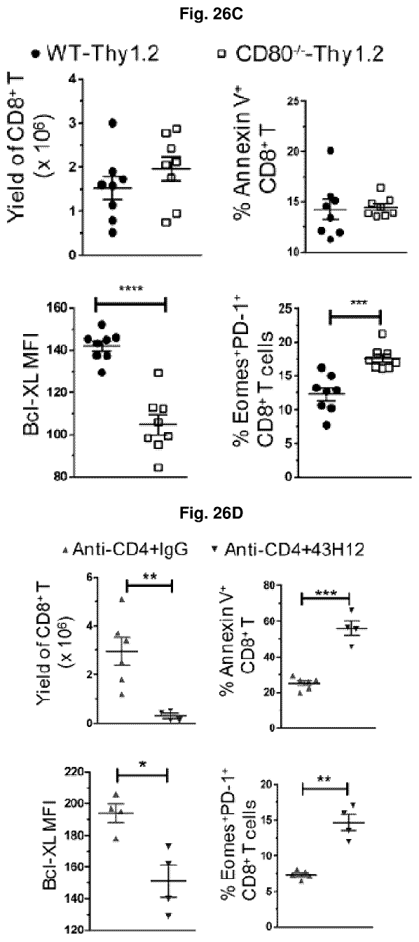

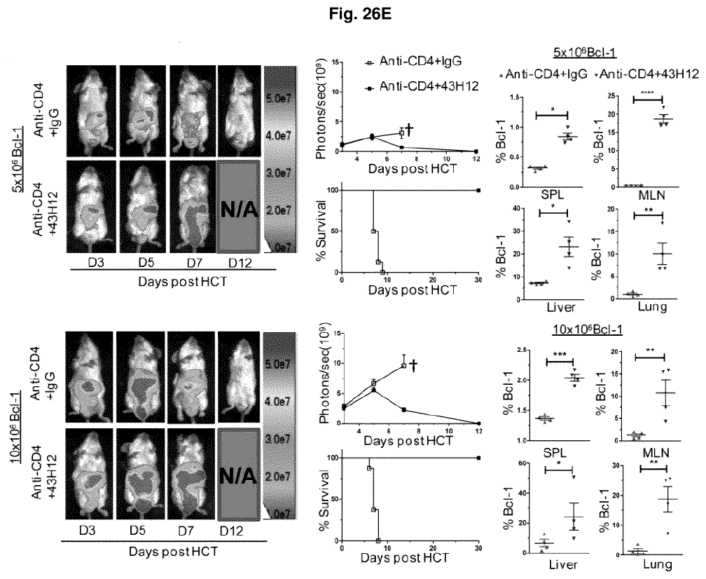

Since donor T cell expression of PD-L1 augments acute GVHD lethality in recipients transplanted with both CD4+ and CD8+ T cells (30), the effect of donor CD8+ T expression of PD-L1 in the expansion and GVL activity of CD8+ T cells in GVHD-free anti-CD4-treated recipients was evaluated. Anti-CD4-treatment significantly upregulated CD8+ T cell expression of PD-L1 in the spleen and liver but not in the colon (FIGS. 26A & 27A). Anti-CD4-treatment also significantly upregulated CD8+ T cell expression of PD-1 and CD80 immediately after HCT (FIGS. 26A & 27A). CD8+ T cells in the spleen had highest expression of PD-L1 and CD80, with the lowest expression of PD-1. In contrast, CD8+ T cells in the colon had the lowest expression of PD-L1 and CD80, with the highest expression of PD-1. The pattern for CD8+ T cells in the liver fell in between, as indicted by the ratio of PD-1/CD80 (FIG. 26A). Consistent with a previous report (52), it was found that non-T cells such as CD11c+ DCs and CD11b/Gr-1+ myeloid cells in the spleen expressed much higher levels of PD-L1 as compared to those in the liver and colon. Anti-CD4-treatment did not significantly change the high expression of PD-L1 by non-T cells in the spleen (FIG. 28). Therefore, the impact of donor CD8+ T PD-L1 and CD80 interactions on donor CD8+ T expansion and GVL effects was tested.

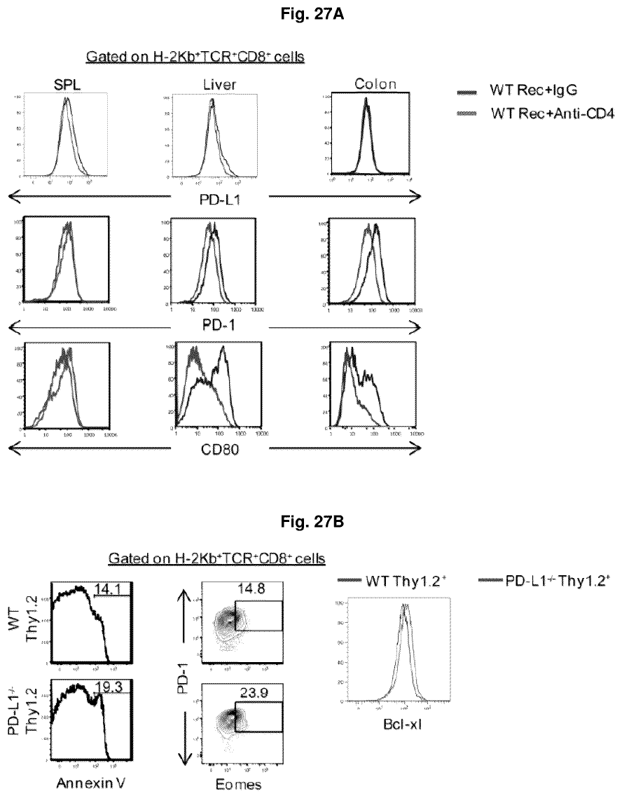

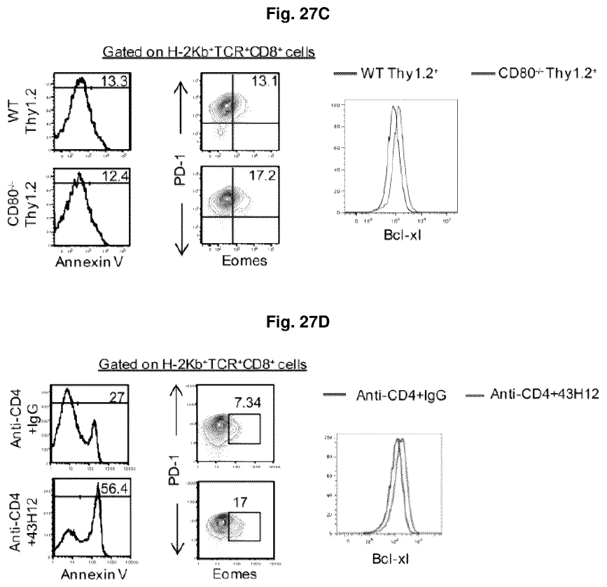

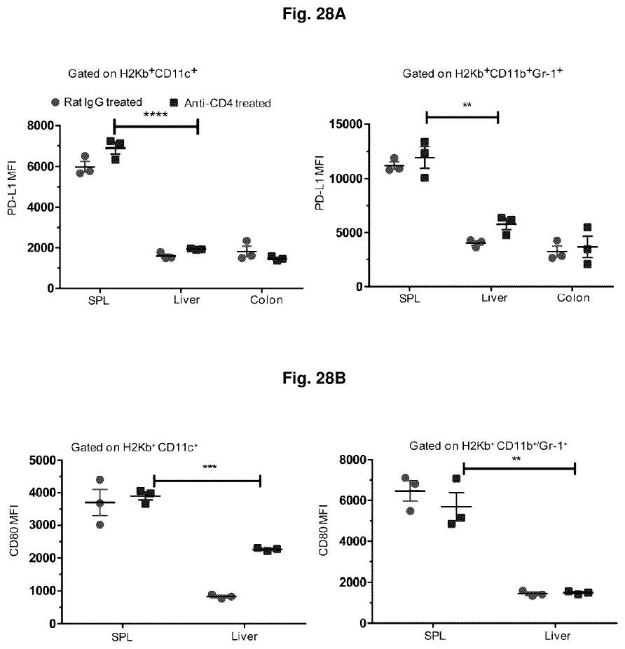

Transplantation of sorted Thy1.2+ T cells from PD-L1−/− C57BL/6 donors and TCD-BM cells from wild-type C57BL/6 donors led to marked reduction of donor CD8+ T expansion in the spleen of anti-CD4-treated recipients immediately after HCT, as compared to Thy1.2+ T cells from WT donors (P<0.001, FIG. 26B). This finding was associated with increased apoptosis, down-regulated expression of BCL-XL, and increased percentage of CD8+ T cells that express PD-1 and Eomes (P<0.01, FIGS. 26B and 27B). Similarly, transplantation of Thy1.2+ T cells from CD80−/− donors and TCD-BM cells from wild-type donors also led to significant down-regulation of BCL-XL and an increased percentage of CD8+ T cells that express PD-1 and Eomes (P<0.01) as compared to T cells from WT donors, although expression of annexin V and expansion of the CD8+ T cells were similar in the two groups (FIGS. 26C and 27C). These results indicate that expression of PD-L1 and CD80 by donor CD8+ T cells are both required in order to augment their survival and expansion in the spleen of anti-CD4-treated recipients immediately after HCT.

To further evaluate the role of PD-L1/CD80 interaction on CD8+ T cell survival and expansion, an anti-PD-L1 mAb (43H12) that specifically blocks PD-L1/CD80 interaction without interfering with PD-L1/PD-1 interaction was used (26). The 43H12 mAb was injected i.p. into anti-CD4-treated WT recipients on days 0 and 2 after HCT. As compared to control IgG treatment, blockade of PD-L1/CD80 interaction also markedly decreased donor CD8+ T cell expansion in the spleen. This finding was associated with augmented apoptosis, reduced expression of BCL-XL, and increased percentage of Eomes+PD-1+ cells (FIGS. 26D and 27D). Taken together, these results indicate that donor CD8+ T-T PD-L1/CD80 interactions play a critical role in augmenting donor CD8+ T survival and expansion in the spleen of anti-CD4-treated recipients immediately after HCT.

Finally, the impact of PD-L1/CD80 interaction on GVL activity in anti-CD4-treated recipients was evaluated. Since BCL1/luc+ tumor cells in anti-CD4-treated recipients were eliminated within 12 days after HCT without relapse by 100 days after HCT (FIG. 6C), the tumor load immediately after HCT in recipients with or without blockade of PD-L1/CD80 interactions via 43H12 mAb treatment on days 0 and 2 after HCT was compared. Anti-CD4-treated recipients were challenged with 5×106 and 10×106 BCL1/Luc cells. Treatment with anti-PD-L1 (43H12) significantly augmented tumor growth as visualized with in vivo BLI (P<0.01, FIG. 26E). Although all (8/8) of anti-CD4-treated recipients eliminated tumor cells before day 12 after HCT, blockade of PD-L1/CD80 augmented tumor growth, as indicated by in vivo BLI, resulting in death of all (8/8) recipients given 5×106 or 10×106 BCL1/Luc cells by day ˜10 after HCT (FIG. 26E). 43H12 mAb treatment also markedly increased the tumor load in the spleen, mesenteric lymph nodes, liver and lungs at 7 days after HCT. Taken together, these results show that donor CD8+ T cell expression of PD-L1 and its interaction with CD80 augments donor CD8+ T survival and expansion in the spleen, resulting in strong GVL activity without causing GVHD immediately after HCT in anti-CD4-treated recipients.

### Example 8: Effects of Depletion of Donor CD4+ T Cells on CD8+ T Cells Expansion in Thymus

This example demonstrates that depletion of donor CD4+ T cells immediately after HCT augmented thymic infiltrating CD8+ T cell anergy.

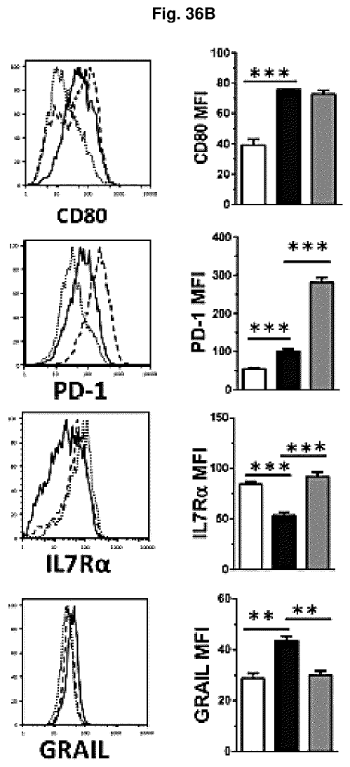

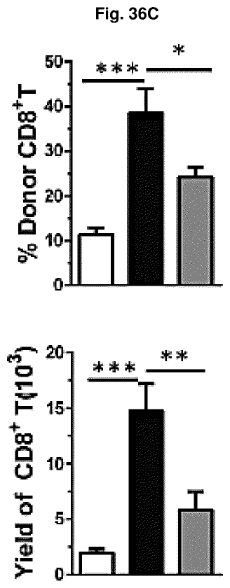

The impact of host-tissue expression of PD-L1 on donor CD8+ T cell expansion in the thymus was evaluated. On day 7 after HCT, the number of thymic mononuclear cells was higher in anti-CD4 treated WT recipients than in rat IgG-treated WT recipients (p<0.01, FIG. 36A). This increase was attenuated in anti-CD4-treated PD-L1−/− recipients (p<0.05), suggesting that in vivo depletion of CD4+ T cells reduced CD8+ T cell-mediated thymus damage in a host-tissue PD-L1-dependent manner. Anti-CD4 treatment increased expression of CD80, PD-1 and GRAIL and decreased expression of IL-7Rα by CD8+ T cells infiltrating the thymus (FIG. 36B). In the absence of recipient PD-L1, expression of GRAIL was not upregulated (FIG. 36B), expression of IL-7Rα was not down-regulated (FIG. 36B), and the increase in number of CD8+ T cells infiltrating the thymus induced by anti-CD4 treatment was attenuated (FIG. 36C).

These results indicate that, in the absence of donor CD4+ T cells, thymic infiltrating CD8+ T cell interaction with host tissue PD-L1 via CD80 and PD-1 lead to donor CD8+ T cell proliferation and development of anergy, such that the accumulation of infiltrating CD8+ T cells did not cause thymic tissue damage.

### Example 9: Effects of Anti-IL-2 Antibody on GVHD

This example demonstrates that injection of an anti-IL-2 mAb after HCT prevented acute GVHD in BALB/c recipients with C57BL/6 transplants.

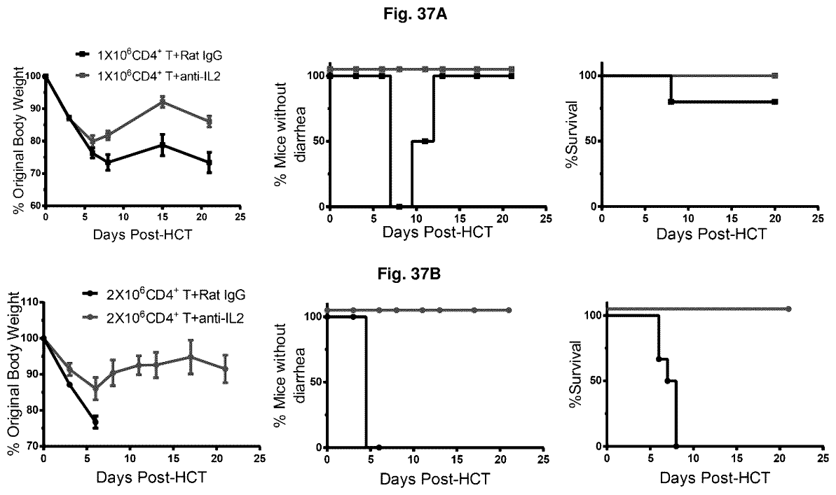

As shown in FIGS. 37A and 37B, an injection of an anti-IL-2 mAb significantly improved body weight, diarrhea, and survival of the mice tested, comparing to control mice received IgG only. These results indicate that anti-IL-2 antibody is effective in preventing acute GVHD.

As stated above, the foregoing are merely intended to illustrate the various embodiments of the present invention. As such, the specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Appelbaum, F. R. 2001. Haematopoietic cell transplantation as immunotherapy. Nature 411:385-389.
2. Martin, P. J. 1993. Donor CD8 cells prevent allogeneic marrow graft rejection in mice: potential implications for marrow transplantation in humans. J Exp Med 178:703-712.
3. Ferrara, J. L., Levine, J. E., Reddy, P., and Holler, E. 2009. Graft-versus-host disease. Lancet 373:1550-1561.
4. Shlomchik, W. D. 2007. Graft-versus-host disease. Nat Rev Immunol 7:340-352.
5. Zeng, D., Lewis, D., Dejbakhsh-Jones, S., Lan, F., Garcia-Ojeda, M., Sibley, R., and Strober, S. 1999. Bone marrow NK1.1(−) and NK1.1(+) T cells reciprocally regulate acute graft versus host disease. J Exp Med 189:1073-1081.
6. Blazar, B. R., Murphy, W. J., and Abedi, M. 2012. Advances in graft-versus-host disease biology and therapy. Nat Rev Immunol 12:443-458.
7. Ito, M., and Shizuru, J. A. 1999. Graft-vs.-lymphoma effect in an allogeneic hematopoietic stem cell transplantation model. Biol Blood Marrow Transplant 5:357-368.
8. Sung, A. D., and Chao, N. J. 2013. Concise review: acute graft-versus-host disease: immunobiology, prevention, and treatment. Stem Cells Transl Med 2:25-32.
9. Chakraverty, R., and Sykes, M. 2007. The role of antigen-presenting cells in triggering graft-versus-host disease and graft-versus-leukemia. Blood 110:9-17.
10. Zeng, D., Hoffmann, P., Lan, F., Huie, P., Higgins, J., and Strober, S. 2002. Unique patterns of surface receptors, cytokine secretion, and immune functions distinguish T cells in the bone marrow from those in the periphery: impact on allogeneic bone marrow transplantation. Blood 99:1449-1457.
11. Wu, T., Young, J. S., Johnston, H., Ni, X., Deng, R., Racine, J., Wang, M., Wang, A., Todorov, I., Wang, J., et al. 2013. Thymic damage, impaired negative selection, and development of chronic graft-versus-host disease caused by donor CD4+ and CD8+ T cells. J Immunol 191:488-499.
12. Withers, D. R., Hepworth, M. R., Wang, X., Mackley, E. C., Halford, E. E., Dutton, E. E., Marriott, C. L., Brucklacher-Waldert, V., Veldhoen, M., Kelsen, J., et al. 2016. Transient inhibition of ROR-gammat therapeutically limits intestinal inflammation by reducing TH17 cells and preserving group 3 innate lymphoid cells. Nat Med 22:319-323.
13. Yu, Y., Wang, D., Liu, C., Kaosaard, K., Semple, K., Anasetti, C., and Yu, X. Z. 2011. Prevention of GVHD while sparing GVL effect by targeting Th1 and Th17 transcription factor T-bet and RORgammat in mice. Blood 118:5011-5020.
14. Yi, T., Zhao, D., Lin, C. L., Zhang, C., Chen, Y., Todorov, I., LeBon, T., Kandeel, F., Forman, S., and Zeng, D. 2008. Absence of donor Th17 leads to augmented Th1 differentiation and exacerbated acute graft-versus-host disease. Blood 112:2101-2110.
15. Borsotti, C., Franklin, A. R., Lu, S. X., Kim, T. D., Smith, O. M., Suh, D., King, C. G., Chow, A., Liu, C., Alpdogan, O., et al. 2007. Absence of donor T-cell-derived soluble TNF decreases graft-versus-host disease without impairing graft-versus-tumor activity. Blood 110:783-786.
16. Na, I. K., Lu, S. X., Yim, N. L., Goldberg, G. L., Tsai, J., Rao, U., Smith, O. M., King, C. G., Suh, D., Hirschhorn-Cymerman, D., et al. 2010. The cytolytic molecules Fas ligand and TRAIL are required for murine thymic graft-versus-host disease. J Clin Invest 120:343-356.
17. Schmaltz, C., Alpdogan, O., Muriglan, S. J., Kappel, B. J., Rotolo, J. A., Ricchetti, E. T., Greenberg, A. S., Willis, L. M., Murphy, G. F., Crawford, J. M., et al. 2003. Donor T cell-derived TNF is required for graft-versus-host disease and graft-versus-tumor activity after bone marrow transplantation. Blood 101:2440-2445.
18. Graubert, T. A., Russell, J. H., and Ley, T. J. 1996. The role of granzyme B in murine models of acute graft-versus-host disease and graft rejection. Blood 87:1232-1237.
19. Graubert, T. A., DiPersio, J. F., Russell, J. H., and Ley, T. J. 1997. Perforin/granzyme-dependent and independent mechanisms are both important for the development of graft-versus-host disease after murine bone marrow transplantation. J Clin Invest 100:904-911.
20. Chen, L., and Flies, D. B. 2013. Molecular mechanisms of T cell co-stimulation and co-inhibition. Nat Rev Immunol 13:227-242.
21. Butte, M. J., Keir, M. E., Phamduy, T. B., Sharpe, A. H., and Freeman, G. J. 2007. Programmed death-1 ligand 1 interacts specifically with the B7-1 costimulatory molecule to inhibit T cell responses. Immunity 27:111-122.
22. Dong, H., Zhu, G., Tamada, K., and Chen, L. 1999. B7-H1, a third member of the B7 family, co-stimulates T-cell proliferation and interleukin-10 secretion. Nat Med 5:1365-1369.
23. Yi, T., Li, X., Yao, S., Wang, L., Chen, Y., Zhao, D., Johnston, H. F., Young, J. S., Liu, H., Todorov, I., et al. 2011. Host APCs augment in vivo expansion of donor natural regulatory T cells via B7H1/B7.1 in allogeneic recipients. J Immunol 186:2739-2749.
24. Schildberg, F. A., Klein, S. R., Freeman, G. J., and Sharpe, A. H. 2016. Coinhibitory Pathways in the B7-CD28 Ligand-Receptor Family. Immunity 44:955-972.
25. Keir, M. E., Butte, M. J., Freeman, G. J., and Sharpe, A. H. 2008. PD-1 and its ligands in tolerance and immunity. Annu Rev Immunol 26:677-704.
26. Park, J. J., Omiya, R., Matsumura, Y., Sakoda, Y., Kuramasu, A., Augustine, M. M., Yao, S., Tsushima, F., Narazaki, H., Anand, S., et al. 2010. B7-H1/CD80 interaction is required for the induction and maintenance of peripheral T-cell tolerance. Blood 116:1291-1298.
27. Li, X., Deng, R., He, W., Liu, C., Wang, M., Young, J., Meng, Z., Du, C., Huang, W., Chen, L., et al. 2012. Loss of B7-H1 expression by recipient parenchymal cells leads to expansion of infiltrating donor CD8+ T cells and persistence of graft-versus-host disease. J Immunol 188:724-734.
28. Saha, A., Aoyama, K., Taylor, P. A., Koehn, B. H., Veenstra, R. G., Panoskaltsis-Mortari, A., Munn, D. H., Murphy, W. J., Azuma, M., Yagita, H., et al. 2013. Host programmed death ligand 1 is dominant over programmed death ligand 2 expression in regulating graft-versus-host disease lethality. Blood 122:3062-3073.
29. Yi, T., Chen, Y., Wang, L., Du, G., Huang, D., Zhao, D., Johnston, H., Young, J., Todorov, I., Umetsu, D. T., et al. 2009. Reciprocal differentiation and tissue-specific pathogenesis of Th1, Th2, and Th17 cells in graft-versus-host disease. Blood 114:3101-3112.
30. Saha, A., O'Connor, R. S., Thangavelu, G., Lovitch, S. B., Dandamudi, D. B., Wilson, C. B., Vincent, B. G., Tkachev, V., Pawlicki, J. M., Furlan, S. N., et al. 2016. Programmed death ligand-1 expression on donor T cells drives graft-versus-host disease lethality. J Clin Invest 126:2642-2660.
31. Deng, R., Cassady, K., Li, X., Yao, S., Zhang, M., Racine, J., Lin, J., Chen, L., and Zeng, D. 2015. B7H1/CD80 interaction augments PD-1-dependent T cell apoptosis and ameliorates graft-versus-host disease. J Immunol 194:560-574.
32. Flutter, B., Edwards, N., Fallah-Arani, F., Henderson, S., Chai, J. G., Sivakumaran, S., Ghorashian, S., Bennett, C. L., Freeman, G. J., Sykes, M., et al. 2010. Nonhematopoietic antigen blocks memory programming of alloreactive CD8+ T cells and drives their eventual exhaustion in mouse models of bone marrow transplantation. J Clin Invest 120:3855-3868.
33. Asakura, S., Hashimoto, D., Takashima, S., Sugiyama, H., Maeda, Y., Akashi, K., Tanimoto, M., and Teshima, T. 2010. Alloantigen expression on non-hematopoietic cells reduces graft-versus-leukemia effects in mice. J Clin Invest 120:2370-2378.
34. Habicht, A., Kewalaramani, R., Vu, M. D., Demirci, G., Blazar, B. R., Sayegh, M. H., and Li, X. C. 2007. Striking dichotomy of PD-L1 and PD-L2 pathways in regulating alloreactive CD4(+) and CD8(+) T cells in vivo. Am J Transplant 7:2683-2692.
35. Edinger, M., Hoffmann, P., Ermann, J., Drago, K., Fathman, C. G., Strober, S., and Negrin, R. S. 2003. CD4+CD25+ regulatory T cells preserve graft-versus-tumor activity while inhibiting graft-versus-host disease after bone marrow transplantation. Nat Med 9:1144-1150.
36. Zhang, C., Lou, J., Li, N., Todorov, I., Lin, C. L., Cao, Y. A., Contag, C. H., Kandeel, F., Forman, S., and Zeng, D. 2007. Donor CD8+ T cells mediate graft-versus-leukemia activity without clinical signs of graft-versus-host disease in recipients conditioned with anti-CD3 monoclonal antibody. J Immunol 178:838-850.
37. Matte-Martone, C., Venkatesan, S., Tan, H. S., Athanasiadis, I., Chang, J., Pavisic, J., and Shlomchik, W. D. 2011. Graft-versus-leukemia (GVL) against mouse blast-crisis chronic myelogenous leukemia (BC-CML) and chronic-phase chronic myelogenous leukemia (CP-CML): shared mechanisms of T cell killing, but programmed death ligands render CP-CML and not BC-CML GVL resistant. J Immunol 187:1653-1663.
38. Sykes, M., Szot, G. L., Nguyen, P. L., and Pearson, D. A. 1995. Interleukin-12 inhibits murine graft-versus-host disease. Blood 86:2429-2438.
39. Hatano, R., Ohnuma, K., Yamamoto, J., Dang, N. H., Yamada, T., and Morimoto, C. 2013. Prevention of acute graft-versus-host disease by humanized anti-CD26 monoclonal antibody. Br J Haematol 162:263-277.
40. Matsushima, K., Ueha, S., and Shono, Y. 2015. Immunological reconstitution promoter or prophylactic agent for infections each of which maintains graft-versus-tumor effect. Google Patents.
41. Hill, G. R., and Ferrara, J. L. 2000. The primacy of the gastrointestinal tract as a target organ of acute graft-versus-host disease: rationale for the use of cytokine shields in allogeneic bone marrow transplantation. Blood 95:2754-2759.
42. Yi, T., Chen, Y., Wang, L., Du, G., Huang, D., Zhao, D., Johnston, H., Young, J., Todorov, I., Umetsu, D., et al. 2009. Reciprocal differentiation and tissue-specific pathogenesis of Th1, Th2, and Th17 cells in graft versus host disease. Blood 114:3101-3112.
43. Choi, J., Ziga, E. D., Ritchey, J., Collins, L., Prior, J. L., Cooper, M. L., Piwnica-Worms, D., and DiPersio, J. F. 2012. IFNgammaR signaling mediates alloreactive T-cell trafficking and GVHD. Blood 120:4093-4103.
44. Coghill, J. M., Sarantopoulos, S., Moran, T. P., Murphy, W. J., Blazar, B. R., and Serody, J. S. 2011. Effector CD4+ T cells, the cytokines they generate, and GVHD: something old and something new. Blood 117:3268-3276.
45. Koyama, M., Cheong, M., Markey, K. A., Gartlan, K. H., Kuns, R. D., Locke, K. R., Lineburg, K. E., Teal, B. E., Leveque-El Mouttie, L., Bunting, M. D., et al. 2015. Donor colonic CD103+ dendritic cells determine the severity of acute graft-versus-host disease. J Exp Med 212:1303-1321.
46. Eriguchi, Y., Takashima, S., Oka, H., Shimoji, S., Nakamura, K., Uryu, H., Shimoda, S., Iwasaki, H., Shimono, N., Ayabe, T., et al. 2012. Graft-versus-host disease disrupts intestinal microbial ecology by inhibiting Paneth cell production of alpha-defensins. Blood 120:223-231.
47. Hanash, A. M., Dudakov, J. A., Hua, G., O'Connor, M. H., Young, L. F., Singer, N. V., West, M. L., Jenq, R. R., Holland, A. M., Kappel, L. W., et al. 2012. Interleukin-22 protects intestinal stem cells from immune-mediated tissue damage and regulates sensitivity to graft versus host disease. Immunity 37:339-350.
48. Pearce, E. L., Mullen, A. C., Martins, G. A., Krawczyk, C. M., Hutchins, A. S., Zediak, V. P., Banica, M., DiCioccio, C. B., Gross, D. A., Mao, C. A., et al. 2003. Control of effector CD8+ T cell function by the transcription factor Eomesodermin. Science 302:1041-1043.
49. Paley, M. A., Kroy, D. C., Odorizzi, P. M., Johnnidis, J. B., Dolfi, D. V., Barnett, B. E., Bikoff, E. K., Robertson, E. J., Lauer, G. M., Reiner, S. L., et al. 2012. Progenitor and terminal subsets of CD8+ T cells cooperate to contain chronic viral infection. Science 338:1220-1225.
50. Intlekofer, A. M., Takemoto, N., Wherry, E. J., Longworth, S. A., Northrup, J. T., Palanivel, V. R., Mullen, A. C., Gasink, C. R., Kaech, S. M., Miller, J. D., et al. 2005. Effector and memory CD8+ T cell fate coupled by T-bet and eomesodermin. Nat Immunol 6:1236-1244.
51. Carbotti, G., Barisione, G., Airoldi, I., Mezzanzanica, D., Bagnoli, M., Ferrero, S., Petretto, A., Fabbi, M., and Ferrini, S. 2015. IL-27 induces the expression of IDO and PD-L1 in human cancer cells. Oncotarget 6:43267-43280.
52. Michonneau, D., Sagoo, P., Breart, B., Garcia, Z., Celli, S., and Bousso, P. 2016. The PD-1 Axis Enforces an Anatomical Segregation of CTL Activity that Creates Tumor Niches after Allogeneic Hematopoietic Stem Cell Transplantation. Immunity 44:143-154.
53. Socie, G., and Ritz, J. 2014. Current issues in chronic graft-versus-host disease. Blood 124:374-384.
54. Blazar, B. R., Carreno, B. M., Panoskaltsis-Mortari, A., Carter, L., Iwai, Y., Yagita, H., Nishimura, H., and Taylor, P. A. 2003. Blockade of programmed death-1 engagement accelerates graft-versus-host disease lethality by an IFN-gamma-dependent mechanism. J Immunol 171:1272-1277.
55. Yang, Y. G., Qi, J., Wang, M. G., and Sykes, M. 2002. Donor-derived interferon gamma separates graft-versus-leukemia effects and graft-versus-host disease induced by donor CD8 T cells. Blood 99:4207-4215.
56. Jasperson, L. K., Bucher, C., Panoskaltsis-Mortari, A., Mellor, A. L., Munn, D. H., and Blazar, B. R. 2009. Inducing the tryptophan catabolic pathway, indoleamine 2,3-dioxygenase (IDO), for suppression of graft-versus-host disease (GVHD) lethality. Blood 114:5062-5070.
57. Shono, Y., Ueha, S., Wang, Y., Abe, J., Kurachi, M., Matsuno, Y., Sugiyama, T., Nagasawa, T., Imamura, M., and Matsushima, K. 2010. Bone marrow graft-versus-host disease: early destruction of hematopoietic niche after MHC-mismatched hematopoietic stem cell transplantation. Blood 115:5401-5411.
58. Hoffmann, P., Ermann, J., Edinger, M., Fathman, C. G., and Strober, S. 2002. Donor-type CD4(+)CD25(+) regulatory T cells suppress lethal acute graft-versus-host disease after allogeneic bone marrow transplantation. J Exp Med 196:389-399.
59. Carter, L., Fouser, L. A., Jussif, J., Fitz, L., Deng, B., Wood, C. R., Collins, M., Honjo, T., Freeman, G. J., and Carreno, B. M. 2002. PD-1:PD-L inhibitory pathway affects both CD4(+) and CD8(+) T cells and is overcome by IL-2. Eur J Immunol 32:634-643.
60. Pulko, V., Harris, K. J., Liu, X., Gibbons, R. M., Harrington, S. M., Krco, C. J., Kwon, E. D., and Dong, H. 2011. B7-h1 expressed by activated CD8 T cells is essential for their survival. J Immunol 187:5606-5614.
61. Mueller, S. N., Vanguri, V. K., Ha, S. J., West, E. E., Keir, M. E., Glickman, J. N., Sharpe, A. H., and Ahmed, R. 2010. PD-L1 has distinct functions in hematopoietic and nonhematopoietic cells in regulating T cell responses during chronic infection in mice. J Clin Invest 120:2508-2515.
62. Martin, P. J., Rowley, S. D., Anasetti, C., Chauncey, T. R., Gooley, T., Petersdorf, E. W., van Burik, J. A., Flowers, M. E., Storb, R., Appelbaum, F. R., et al. 1999. A phase I-II clinical trial to evaluate removal of CD4 cells and partial depletion of CD8 cells from donor marrow for HLA-mismatched unrelated recipients. Blood 94:2192-2199.
63. Li, N., Chen, Y., He, W., Yi, T., Zhao, D., Zhang, C., Lin, C. L., Todorov, I., Kandeel, F., Forman, S., et al. 2009. Anti-CD3 preconditioning separates GVL from GVHD via modulating host dendritic cell and donor T-cell migration in recipients conditioned with TBI. Blood 113:953-962.
64. Fowler, D. H., Breglio, J., Nagel, G., Eckhaus, M. A., and Gress, R. E. 1996. Allospecific CD8+ Tc1 and Tc2 populations in graft-versus-leukemia effect and graft-versus-host disease. J Immunol 157:4811-4821.
65. Yang, Y. G., Dey, B. R., Sergio, J. J., Pearson, D. A., and Sykes, M. 1998. Donor-derived interferon gamma is required for inhibition of acute graft-versus-host disease by interleukin 12. J Clin Invest 102:2126-2135.
66. Yang, Y., Wang, H., Yu, H., Yeap, B. Y., Liang, T., Wang, G., Cheng, T., and Yang, Y. G. 2011. IFN-gamma promotes graft-versus-leukemia effects without directly interacting with leukemia cells in mice after allogeneic hematopoietic cell transplantation. Blood 118:3721-3724.
67. Yamazaki, T., Akiba, H., Iwai, H., Matsuda, H., Aoki, M., Tanno, Y., Shin, T., Tsuchiya, H., Pardoll, D. M., Okumura, K., et al. 2002. Expression of programmed death 1 ligands by murine T cells and APC. J Immunol 169:5538-5545.
68. Donaldson, J., Zer C., Avery K., Bzymek K., Horne D., and Williams J. 2013. Identification and grafting of a unique peptide-binding site in the Fab frame work of monoclonal antibodies. PNAS 110 (43): 17456-17461.
69. Sykes M., Abraham V., Harty M. and Pearson D. 1993. IL-2 reduces graft-versus-host disease and preserves a graft-versus-leukemia effect by selectively inhibiting CD4+ T cell activity. J. Immunology 150: 197-205.
70. Kekre N., Antin J H. 2016. Emerging drugs for graft-versus-host disease. Expert Opin. Emerg. Drugs 21(2): 209-218.
71. Scandiuzzi, L., Ghosh, K., Hofmeyer, K. A., Abadi, Y. M., Lazar-Molnar, E., Lin, E. Y., Liu, Q., Jeon, H., Almo, S. C., Chen, L., et al. 2014. Tissue-expressed B7-H1 critically controls intestinal inflammation. Cell Rep 6:625-632.
72. Sakuishi, K., Apetoh, L., Sullivan, J. M., Blazar, B. R., Kuchroo, V. K., and Anderson, A. C. 2010. Targeting Tim-3 and PD-1 pathways to reverse T cell exhaustion and restore anti-tumor immunity. J Exp Med 207:2187-2194.
73. Whiting, C. C., Su, L. L., Lin, J. T., and Fathman, C. G. 2011. GRAIL: a unique mediator of CD4 T-lymphocyte unresponsiveness. FEBS J 278:47-58.
74. Schietinger, A., and Greenberg, P. D. 2014. Tolerance and exhaustion: defining mechanisms of T cell dysfunction. Trends Immunol 35:51-60.
75. Wherry, E. J. 2011. T cell exhaustion. Nat Immunol 12:492-499.
76. Anasetti et al. 1991, Bone Marrow Transplant 7(5): 375-381.
77. Ferrant et al. 1995, Bone Marrow Transplant 16(4): 577-581.

1. A method for preventing or treating graft-versus-host disease (GVHD) while preserving graft versus leukemia/lymphoma (GVL) effects in a subject receiving hematopoietic cell transplantation (HCT), comprising administering one or more doses of a therapeutically effective amount of a therapeutic agent to the subject to temporarily deplete CD4+ T cells in vivo or to temporarily reduce serum IL-2 in the subject, wherein the therapeutic agent is administered to the subject simultaneously with HCT, immediately before HCT, or immediately after HCT.

2. The method of claim 1, wherein the therapeutic agent includes an anti-CD4 antibody, an anti-CD4-meditope-immunotoxin, an anti-IL-2 antibody, an agent blocking IL-2R, and a PD-L1-Ig.

3. The method of claim 2, wherein the anti-CD4 antibody is a monoclonal antibody or a humanized antibody.

4. The method of claim 2, wherein the anti-IL-2 antibody is a monoclonal antibody or a humanized antibody.

5. The method of claim 1, further comprising administering one or more doses of IFN-γ to the subject.

6. The method of claim 1, wherein a first dose of the therapeutic agent is administered to the subject up to about 10 days before HCT.

7. The method of claim 1, wherein a first dose of the therapeutic agent is administered to the subject about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3 days, about 4 days, about 5 days, about 6 days, about 7 days, about 8 days, about 9 days, or about 10 days, before HCT.

8. The method of claim 1, wherein a first dose of the therapeutic agent is administered to the subject any time up to about 6 weeks after HCT.

9. The method of claim 1, wherein a first dose of the therapeutic agent is administered to the subject about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 5 weeks, or about 6 weeks, after HCT.

10. The method of claim 1, wherein a single dose of the therapeutic agent is administered to the subject to effectively prevent acute GVHD.

11. The method of claim 10, wherein the single dose of the therapeutic agent is administered to the subject on the same day of receiving HCT.

12. The method of claim 1, wherein two or more doses of the therapeutic agent are administered to the subject to effectively prevent both acute GVHD and chronic GVHD.

13. The method of claim 12, wherein three doses of the therapeutic agent are administered to the subject.

14. The method of claim 13, wherein the three doses of the therapeutic agent are administered to the subject within one month of receiving HCT.

15. The method of claim 13, wherein the three doses of the therapeutic agent are administered to the subject at one-week or two-week intervals.

16. The method of claim 13, wherein the first dose of the therapeutic agent is administered to the subject on the same day of receiving HCT.

17. A method of in vivo expanding CD8+ T cells in a subject receiving HCT, comprising administering one or more doses of a therapeutically effective amount of a therapeutic agent to the subject to temporarily deplete CD4+ T cells in vivo or to temporarily reduce serum IL-2 in the subject, wherein the therapeutic agent is administered to the subject simultaneously with HCT, immediately before HCT, or immediately after HCT.

18. The method of claim 17, wherein the therapeutic agent includes an anti-CD4 antibody, an anti-CD4-meditope-immunotoxin, an anti-IL-2 antibody, an agent blocking IL-2R, and a PD-L1-Ig.

19. The method of claim 18, wherein the anti-CD4 antibody is a monoclonal antibody or a humanized antibody.

20. The method of claim 18, wherein the anti-IL-2 antibody is a monoclonal antibody or a humanized antibody.

21-49. (canceled)
